(19) United States
(12) Patent Application Publication
(10) Pub. No.: US 2013/0097729 A1
(43) Pub. Date: Apr. 18, 2013

(54) Aphicidal Toxins and Methods

(71) Applicants: Bryony Claire Bonning, Ames, IA (US); Sijun Liu, Ames, IA (US); Huarong Li, Zionsville, IN (US)

(72) Inventors: Bryony Claire Bonning, Ames, IA (US); Sijun Liu, Ames, IA (US); Huarong Li, Zionsville, IN (US)

(21) Appl. No.: 13/492,779

(22) Filed: Jun. 8, 2012

Related U.S. Application Data
(60) Provisional application No. 61/494,559, filed on Jun. 8, 2011.

(51) Int. Cl. C12N 15/82

(52) U.S. Cl. CPC C12N 15/8286
USPC 800/279; 530/350; 536/23.4; 435/320.1; 800/302; 514/4.5; 435/419

(57) ABSTRACT

Provided are chimeric aphicidal and insecticidal toxin proteins comprising peptide, peptide multimer or fusion protein containing such peptide which binds to the gut of sap-sucking insects, e.g., aphids, thrips, leafhoppers, or other target interest. When bound, this peptide mediates the binding of the chimeric aphicidal or other insecticidal protein to the target insect gut. Also described are coding sequences, vectors, and transgenic plants genetically modified to contain and express such aphicidal or insecticidal proteins. Thus, the use of such transgenic plants reduces economic loss due to feeding by the target insect and also reduces loss due to plant diseases spread by the target insect.

### CROSS REFERENCE TO RELATED APPLICATIONS

This application claims the benefit of U.S. Provisional Application No. 61/494,559, filed Jun. 8, 2011, which is incorporated by reference herein to the extent there is no inconsistency with the present disclosure.

### ACKNOWLEDGEMENT OF FEDERAL RESEARCH FUNDING

This invention was made with funding from Contract No. EM-83438801 from the Environmental Protection Agency. The United States government has certain rights in this invention.

### REFERENCE TO SEQUENCE LISTING

The Sequence Listing filed herewith is incorporated by reference to the extent there is no inconsistency with the present disclosure.

### BACKGROUND

This disclosure relates to biological control of aphids and molecular biological methods for doing so, especially using chimeric insecticidal Bacillus thuringiensis toxins engineered to comprise a peptide region which directs binding to the gut of aphids. The sap-sucking insects (Hemiptera), including aphids and plant bugs, currently present one of the biggest challenges for insect pest management in United States agriculture. Aphids are among the most pervasive pests of temperate agriculture and affect almost all agricultural crops. The soybean aphid alone is estimated to account for $1.6 billion in losses over the past decade. Invasive species such as the Russian wheat aphid and the more recently introduced soybean aphid have had a particularly severe impact on U.S. agriculture. Economic losses result from direct feeding on plants, from aphid-transmitted plant viruses, and from production of honeydew which results in growth of harmful sooty molds.

Aphids are among the most economically important pest insects of temperate agriculture, cause major economic losses on almost all crops, and account for a large part of the 13% of agricultural output that is lost to insect pests. For example, aphids are estimated to cause yield losses of 7% in tomato, 22% in potatoes, 27% in cotton and 100% in rapeseed in the absence of control measures. In addition to the major economic losses resulting from aphid feeding, aphids also transmit plant viruses with more than 200 plant viruses vectored by aphids (60, 84).

Management of hemipteran pests relies primarily on the application of environmentally damaging chemical insecticides. However, aphids in particular readily develop insecticide resistance. An alternative approach for aphid management would be use of insect-specific toxins, as described herein.

Because of the pervasive nature of aphid damage on a wide variety of crops, the study of aphid resistance genes has received a good deal of attention (Cevik & King, 2002, Pascal et al., 2002, Goggin et al., 1998, Klingler et al., 2001, Wang et al., 2001, Hartman, 2004; Rouf Mian, 2008). Aphid resistant maize and wheat lines developed by traditional plant breeding techniques have had some success in limiting aphid damage (Auclair, 1989, Thackray et al., 1990, Walter & Brunson, 1946, Quisenberry & Schotzko, 1994). However, transgenic aphid resistance would free breeders from the narrow range of germplasm that contains natural resistance genes, which would be a major advantage. Although transgenic plants that express plant lectins confer resistance to aphids (Hilder et al., 1995, Gatehouse et al., 1996), lectins have not been adopted for aphid management purposes.

The soybean aphid (Aphis glycines) is an invasive species that originates from Asia. In North America, the soybean aphid alternates between sexual reproduction on primary hosts (buckthorn trees, Rhamnus spp.) and asexual reproduction on the secondary host (soybean, Glycines max) (Ragsdale et al., 2004). Populations can double every 6 to 7 days, with some 15 generations within one growing season (Ragsdale et al., 2007). Since the first discovery of the soybean aphid in the U.S. in 2000 (Ragsdale et al. 2011), it has spread throughout the north-central region and into parts of Canada, with an estimated $1.6 billion spent on management. In addition, without effective plant-based management of the soybean aphid, $3.6 to $4.9 billion could be lost annually in soybean production, depending on insecticide costs, the severity of the aphid outbreak, and the price of soybeans (Kim et al., 2008). Although natural resistance genes have been identified in soybean varieties, they are not effective against all soybean aphid populations (Kim et al., 2008, Hill et al., 2010). There is an urgent need for a sustainable management alternative to the use of classical chemical insecticides against the soybean aphid.

The use of transgenic crops expressing insecticidal proteins from Bacillus thuringiensis (Bt) has become a primary approach for lepidopteran and coleopteran pest management (Shelton et al., 2002, Christou et al., 2006). The use of Bt transgenic crops has benefited both the growers through crop protection and the environment by reducing the use of environmentally damaging, classical chemical insecticides (Shelton et al., 2002). Hemipteran pests with piercing and sucking mouthparts are not particularly susceptible to the effects of Bt toxins, which may result from lack of exposure to B. thuringiensis, which exists in the soil and on the surface of foliage. Hence, there has been no natural selection for toxicity of Bt toxins to the Hemiptera (Schnepf et al., 1998). Indeed the deleterious impact of aphids and plant bugs on Bt cotton and Bt corn is increasing, thereby compromising the success of the Bt technology because of the need to apply chemical insecticides for hemipteran pests (Greene et al., 1999, Greene et al., 2001).

Taking advantage of a more sustainable approach, toxins derived from the bacterium Bacillus thuringiensis (Bt) have been used successfully for management of other insect pests, but exhibit only low levels of toxicity against the Hemiptera. Transgenic crops expressing Bacillus thuringiensis (Bt) toxins now play a primary role for management of lepidopteran (moth) and coleopteran (beetle) pests. The lack of efficacy of Bt toxins against aphids may result, in part, from the lack of a domain that binds to the aphid gut, which is a critical step in the sequence of events that results in Bt toxicity.

Several reports have demonstrated low levels of toxicity to aphids at high Bt toxin doses: Feeding assays indicated some toxicity of the Bt toxins Cry2, Cry3A, and Cry4 against the potato aphid, Macrosiphum euphorbiae (Walters & English, 1995, Walters et al., 1994). Porcar et al (2009) demonstrated low to moderate toxicity of Cry3A, Cry4Aa and Cry11Aa to the pea aphid with 100% mortality following feeding on 125 to 500 μg/ml Cry4 or Cry11 (Porcar et al., 2009). For comparison with known toxicity against species that are susceptible to Cry toxins, the LC50 of Cry1Ac against Heliothis virescens is around 1 μg/ml (Ali, 2006), and the LC50 of Cry3Aa against Leptinotarsa decemlineata is 3.56 μg/ml (Park, 2009). Analyses of the impact of transgenic plants expressing Cry toxins on aphids gave variable results ranging from minor negative effects on aphid survival and fecundity to significant beneficial effects on aphid populations (Ashouri, 2004a, Ashouri, 2004b, Ashouri et al., 2001, Faria et al., 2007, Mellet & Shoeman, 2007, Raps et al., 2001, Lawo et al., 2009, Burgio et al., 2007).

The physiological basis for the low aphicidal toxicity of two representative Cry toxins has been examined for Cry1Ac and Cry3Aa in the pea aphid gut (Li et al., 2011). The interaction with the aphid varies with the toxin: Cry1Ac was processed by aphid gut proteases to produce active toxin, which bound to the aphid gut epithelium but showed low aphicidal activity. Cry3Aa was incompletely processed and partially degraded in the aphid gut resulting in production of low amounts of active toxin. Active Cry3Aa bound to brush border membrane vesicles (BBMV) proteins and showed a similar low level of toxicity against the pea aphid in bioassays. The mechanisms of Cry toxin action in aphids downstream of toxin binding remain to be explored.

Different models have been proposed to explain the mode of action of insecticidal Cry toxins (Bravo et al., 2004, Jurat-Fuentes & Adang, 2006, Zhang et al., 2006). These models propose a single (Zhang et al., 2006) binding step to cadherin or a mechanism with sequential steps of toxin interaction with insect gut membrane receptors including cadherin-like proteins, GPI anchored aminopeptidase (APN), and alkaline phosphatase (ALP). Multiple interactions are required to produce a toxic effect against the target organism. In this model, monomeric toxin interacts first with the cadherin receptor leading to the formation of a pre-pore oligomer (Gomez, 2002). In the second step this oligomer binds to GPI-anchored receptors, GPI-anchored aminopeptidase N or alkaline phosphatase, leading to insertion of the pre-pore oligomers into the insect gut membrane (Bravo, 2004; Zhuang, 2002). It is believed that specific binding to insect gut receptors is an important step in the mode of action of insecticidal Cry toxins. In several cases, Cry toxin specificity and toxicity correlate with toxin binding to gut brush border membrane receptors in vitro at both qualitative and quantitative levels, although there are some exceptions. After binding, the Cyt toxins cause disruption of the integrity of the membrane.

Activated Cry4Aa consists of three structurally conserved globular domains, domain I of seven α-helical bundles, domain II of antiparallel β-strands and domain III of two antiparallel β-sheets. Cry4Aa exhibits high level of toxicity against Culex and Aedes mosquito larvae and some toxicity to Anopheles mosquito larvae (Poncet et al., 1995). For insertion of the toxin into the target membrane, a4 and 5 hairpin structures is required (Gerber and Shai, 2000). The loop connecting a4 and a5 is responsible for efficient penetration of these two transmembrane helices into the lipid membranes to form lytic pores (Tapaneeyakorn et al., 2005). The integrity of this loop is maintained in by a disulfide bridge (Cys192-Cys199) and a proline ˜rich motif (Boonserm et al., 2006). Cry4Ba (which is very similar to Cry4Aa) has been reported to interact with mosquito cadherin and alkaline phosphatase (Hua et al., 2008; Bayyareddy et al., 2009). For characterization of the motifs of Cry4Aa that function in toxicity, loop replacement studies showed the importance of loop 2 (Boonserm et al., 2006; Howlader et al., 2009) while alanine scanning of all three loops predicted multiple binding sites (Howlader et al., 2010). The presence of loop2 and loop3 in the close vicinity of domain I and domain II appears to be crucial for receptor binding which could disrupt the interface between domain I and domain II and prime domain I for insertion into the membrane (Boonserm et al., 2006). Cry4Aa Domain III shows structural similarity with the N-terminal cellulose binding domain of a protein from Cellulomonas fimi (Johnson et al., 1996) and a xylanase from Clostridum thermocellum (Czjzek et al., 2001) which suggests that domain III may bind to the carbohydrate moiety of a glycoprotein receptor on the target insect membrane. Cry4Aa receptors have not been identified so far, and the importance of sugars in Cry4Aa toxicity has not been demonstrated. However, lectin-like domain III of the lepidopteran-specific Cry1Ac toxin has been shown to bind N-acetylgalactosamine (Burton et al., 1999; Jenkins et al., 1999; Jenkins et al., 2000).

Mehlo et al. fused a Bt toxin (Cry1Ac) with the galactose-binding domain of a lectin (ricin) and demonstrated that the increased ability of the toxin to bind allowed for greater resistance of transgenic maize and rice against insect pests already susceptible to Cry1Ac (specifically the stem borer, Chilo suppressalis, and the cotton leaf worm, Spodoptera littoralis), and also resistance to insects that are not normally susceptible to Cry1Ac (the leafhopper, Cicadulina mbila). Notably, no resistance was detected against the cereal aphid, Rhopalosiphum padi in this study.

There is a long felt need in the art for environmentally friendly and economical methods for reducing damage to both crop and ornamental plants from aphid infestation. The present invention meets this need.

### SUMMARY

There is provided a chimeric insecticidal toxin that specifically binds to a receptor in a sap-sucking insect gut, especially an aphid gut, via a peptide or peptide multimers incorporated within the chimeric insecticidal toxin, for example as an N-terminal extension of the toxin or incorporated within surface domains of the toxin. By mediating the gut binding of the insecticidal protein, acquired by the insect feeding on a plant which expresses the chimeric toxin or to which the chimeric toxin has been topically applied, certain plant pests feeding on the plant leaves are killed, although topical application of a chimeric insecticidal toxin is not considered appropriate for sap sucking-insects. The use of this chimeric toxin applies to insects which feed on plant fluids (sap-sucking insects), including but not limited to, aphids and planthoppers, whiteflies (Hemiptera) and thrips (Thysanoptera). An especially important target is the soybean aphid A. glycines, which attacks soybean and other legume plants, including but not limited to forage crops such as clover and alfalfa.

A specifically exemplified insecticidal toxin modified to contain a gut-binding peptide of a sap-sucking insect is the Cyt2Aa toxin of Bacillus thuringiensis. This toxin has very low insecticidal activity against the green peach aphid Myzus persicae and the pea aphid Acyrthosiphon pisum. Incorporation of at least one gut binding peptide as described herein as an N-terminal extension or an insertion into loops (especially 1, 3 or 4) of Cyt2Aa results in significantly greater insecticidal activity against these two representative hemipteran pests. Incorporation of multiple gut binding peptides (collectively specific to multiple insects) into multiple loops (i.e., into two or more of loops 1, 3 and 4) to expand the range of target insects for a particular chimeric insecticidal toxin is also contemplated. Alternatively, one or more gut-binding peptides can be substituted in place of certain surface loops of an insecticidal protein, including by not limited to a Bt protein such as Cyt2Aa and Cry4. For leafhoppers or other plant pests which feed on xylem, leaf-specific promoters and/or light-activated promoters or other promoters which cause expression in xylem are useful for directing the expression of a chimeric insecticidal toxin as described herein.

Plants susceptible to attack by sap-sucking insects are transformed to express a chimeric toxin as described herein in the fluids of the plant phloem, or xylem, upon which insects such as aphids feed. An aphid feeding on the phloem of a transgenic plant expressing a chimeric toxin provided herein thereby acquires the chimeric toxin in its gut, where the peptide mediates binding of the chimeric toxin, such that the aphid is killed or at least inhibited from feeding, thus providing for at least partial protection of the plant form the relevant plant pest and methods of insect control. Transgenic plants expressing the chimeric toxin including this peptide inhibit aphid feeding on the same and on other plants, lower the incidence of crop damage due to feeding by aphids and other sap-sucking insects and also lower virus infection in a crop of such plants where the virus is aphid transmitted thereby reducing damage in the crop as a whole. For other insects which feed on plant tissue, chimeric insecticidal toxin expression is directed to the appropriate tissue, such as leaf, stem or xylem or constitutive expression of the chimeric insecticidal toxin throughout the plant can be effected.

Gut binding peptides provided herein include the following:

(SEQ ID NO: 1)
Ala Thr Cys Ser Lys Lys Tyr Pro Arg Ser Pro Cys
Met Ala (GBP3.1)
(SEQ ID NO: 2)
Ala Thr Cys Ser ALA Lys Tyr Pro Arg Ser Pro Cys
Met Ala (GBP3.1-K4A)
(SEQ ID NO: 3)
Ala Thr Cys Ser Lys ALA Tyr Pro Arg Ser Pro Cys
Met Ala (GBP3.1-K5A)
(SEQ ID NO: 4)
Ala Thr Cys Ser Lys Lys Tyr Pro Ala Ser Pro Cys
Met Ala (GBP3.1-R8A)
(SEQ ID NO: 5)
Ala Thr Cys Ser Lys Lys Tyr Pro Ser Ser Asp Cys
Gln Ala
(SEQ ID NO: 6)
Ala Thr Cys Ser Lys Lys Tyr Pro Ser Ser Glu Cys
Met Ala
(SEQ ID NO: 7)
Ala Thr Cys Ser Lys Lys Tyr Pro Arg Ser Asp Cys
Met Ala
(SEQ ID NO: 8)
Ala Thr Cys Ser Lys Lys Tyr Pro Ser Ser Pro Cys
Gln Ala
(SEQ ID NO: 9)
Ala Thr Cys Ser Lys Lys Tyr Pro Arg Ser Pro Cys
Gln Ala
(SEQ ID NO: 10)
Ala Gly Cys Ser Lys Lys Tyr Pro Arg Ser Pro Cys
Met Ala
(SEQ ID NO: 11)
Thr Cys Ser Lys Lys Tyr Pro Arg Ser Pro Cys Met
(SEQ ID NO: 12)
Thr Cys Ser Lys Lys Tyr Pro Ser Ser Asp Cys Gln
(SEQ ID NO: 13)
Thr Cys Ser Lys Lys Tyr Pro Ser Ser Glu Cys Met
(SEQ ID NO: 14)
Thr Cys Ser Lys Lys Tyr Pro Arg Ser Asp Cys Met
(SEQ ID NO: 15)
Thr Cys Ser Lys Lys Tyr Pro Ser Ser Pro Cys Gln
(SEQ ID NO: 16)
Thr Cys Ser Lys Lys Tyr Pro Arg Ser Pro Cys Gln
(SEQ ID NO: 17)
Gly Cys Ser Lys Lys Tyr Pro Arg Ser Pro Cys Met
(SEQ ID NO: 18)
Thr Cys Ser Ala Lys Tyr Pro Arg Ser Pro Cys Met
Ala
(SEQ ID NO: 19)
Thr Cys Ser Lys Ala Tyr Pro Arg Ser Pro Cys Met
Ala
(SEQ ID NO: 20)
Thr Cys Ser Lys Lys Tyr Pro Lys Ser Pro Cys Met
Ala

The N- and C-terminal alanine residues of SEQ ID NOs: 1-11 are not required for inhibitory activity. The results of Alanine scanning mutagenesis indicate that the Lys and Arg residues can be replaced by Ala without eliminating gut binding activity.

The sequence of the gut-binding peptide provided herein can be expressed in terms of the following consensus sequence: Xaa₁-Xaa₂-Cys-Ser-Xaa₃-Xaa₃-Tyr-Pro-Xaa₄-Ser-Xaa₅-Cys-Xaa₆-Xaa₇,- wherein Xaa₁ and Xaa₇, independently of one another, can be any amino acid or no amino acid; Xaa₂ is Thr or Gly; Xaa₃ is Lys or Ala, Xaa₄ is Arg or Ser or Ala; Xaa₅ is Asp or Glu or Pro; and Xaa₆ is Met or Gln (SEQ ID NO:21). A nucleotide sequence encoding an amino acid sequence corresponding to this consensus sequence can be incorporated into the coding sequence of a protein to form a fusion protein, especially where it is incorporated at the N-terminus of the protein (preceded by a Met residue to initiate transcription or where the recited sequence of amino acids is preceded by a signal peptide to allow secretion of the gut-binding fusion protein into the sap of a transgenic plant expressing same). Alternatively, such a peptide can be expressed as a peptide multimer (of identical peptide or one or more peptides of sequences fitting the consensus sequence of SEQ ID NO:21). When the target insect is A. pisum, the gut binding peptide is or comprises one of SEQ ID NO:1-20, and it can be SEQ ID NO:1 or SEQ ID NO:4.

The chimeric insect toxin proteins comprising the gut-binding peptides, peptide multimer and fusion proteins containing same provided herein are especially useful for mediating the binding of Bacillus thuringiensis insecticidal toxin to an aphid gut. As a result, aphid feeding and aphid-to-plant transmission of plant pathogenic viruses is inhibited, and thus damage to the plants is reduced. Of particular interest are those plants which are susceptible to feeding by aphid pests and sharing the gut receptor bound by the afore-mentioned peptide. Reduction of feeding by the plant pest results in improved plant yield.

A DNA sequence encoding the peptide of SEQ ID NO:1 lacking the N-terminal and C-terminal alanine residues is ACG TGT AGT AAG AAG TAT CCG CGT TCT CCG TGT ATG (SEQ ID NO:22). It is understood that other synonymous coding sequences can be substituted for SEQ ID NO: 22 in the practice of various embodiments herein. Sequences encoding the other gut binding peptides described herein can be generated using the well-known genetic code, and the skilled artisan can adapt codon usage according to the plant or other organism in which the chimeric insecticidal protein is to be expressed, with reference to readily available information in the art.

A further embodiment encompasses a method of reducing the spread of plant pathogens by providing plants genetically modified to contain within the genome and express the chimeric insecticidal toxin disclosed herein, with the toxin being expressed in plant tissue on which the plant pests that spread the disease feed. For example, a transgenic plant expressing the chimeric insecticidal toxin in phloem tissue of plants attacked by the pea aphid, especially peas and related plants, will cause inhibition of feeding and/or death of the aphid, and thus, transmission of viruses spread by that aphid will reduced, as well as the result of reduced direct damage to the plant due to feeding.

Also provided herein are plants genetically modified to contain within their genome a chimeric insecticidal toxin coding sequence as described herein operably linked to a plant-expressible promoter and expressing that coding sequence in plant tissue subject to feeding by an insect pest. For example, when the plant pest is a sap sucking insect, the chimeric insecticidal toxin is expressed in phloem tissue. Feeding on the plant tissue results in inhibition of feeding by the insect pest or death of the insect pest. In the case of aphids, the chimeric insecticidal toxin is expressed in phloem, and the toxin comprises a peptide portion which mediates binding of the toxin to the insect gut and subsequent damage to the insect. In other embodiments the chimeric insecticidal toxin can be expressed constitutively, in all tissues, or in others, in leaf tissues, as appropriate for the feeding site of the target insect.

In a particular embodiment, the gut binding peptide portion comprises an amino acid sequence set forth in SEQ ID NO:1-20 or matches the consensus sequence set forth in SEQ ID NO:21. An exemplary toxin component is that of Cyt2Aa of B. thuringiensis (see sequences herein, e.g., SEQ ID NO:26) is ingested by insects including aphids, planthoppers, thrips or whiteflies into the gut of the insect, comprising providing a peptide or peptide multimer as part of the chimeric toxin comprising the amino acid sequence of the gut binding peptide set forth in the consensus sequence set forth in SEQ ID NO:21 and insecticidal toxin, and bringing a source of food containing the chimeric insecticidal toxin into contact with the insect under conditions that allow the insect to ingest the food, especially transgenic plant tissue, whereby the chimeric toxin ingested by the insect binds the gut epithelium and insect death ensues. As a result, feeding is reduced and transmission of a pathogen carried by the insect to a susceptible plant is reduced, and incidence and/or severity of disease caused by the pathogen is reduced. The peptides and peptide components of the chimeric insecticidal toxins specifically exemplified herein enable the inhibition of the spread of plant pathogens carried by certain plant pests, including aphids and certain other sap-sucking insects where those pathogens include viruses including but not limited to Luteoviruses, Geminiviruses and Enamoviruses, in particular Pea enation mosaic virus (PEMV), as well as plant pathogenic fungi and bacteria.

Further provided is a method of making a chimeric insecticidal toxin comprising a gut binding peptide portion and a toxin portion, said method comprising the step of identifying a gut binding peptide for a target insect which is not susceptible to naturally occurring Bt by panning a gut binding peptide from a peptide library using target insect gut epithelial tissue or target insect brush border membrane vesicles (BBMV) or target insect receptor protein, and fusing the nucleotide sequence encoding the gut binding peptide in frame with a Bt coding sequence, advantageously as a N-terminal extension or within loop 1, 3 or 4, advantageously at the locations disclosed herein for the incorporation of the GBP3.1 or other gut binding peptide into Cyt2Aa, or into Cry4A or other insecticidal toxin. Insertions or substitutions of a peptide or peptide multimer which bindings to the gut membrane of an insect plant pest of interest can be made to render the chimeric toxin effective against that insect pest of the plant. Then stable introduction of the plant-expressible chimeric toxin gene into the plant and expression of that chimeric toxin gene in the plant allows for at least partial protection of the plant from the targeted insect pest.

### DESCRIPTION OF THE DRAWINGS

FIG. 1 shows Western blot detection of EGFP-labeled peptides that bound to whole aphid brush border membrane vesicles (BBMV) proteins in pull down assays. EGFP-labeled peptides that bound to BBMV proteins were detected in the blot using anti-EGFP antibodies. Positive control, GBP3.1-EGFP. Note that although C6-EGFP binds BBMV, it does not bind to aphid epithelial cells in vivo (see also Liu et al., 2010).

FIG. 2 shows relative binding of GBP3.1-EGFP and GBP3.1 alanine addition mutants to whole aphid BBMV. No binding was detected for EGFP and BBMV (negative controls) in binding experiments (FIG. 1). Band intensities in FIG. 1 were quantified using ImageJ software. Numbers on each bar indicate protein bound (ng) to whole aphid BBMV (10 μg starting material). GBP3.1 mutations: k-a, K5A; K-A, K4A; R-A, R8A.

FIG. 3 illustrates Western blot detection of EGFP-labeled peptides that bound to pea aphid gut BBMV proteins in pull down assays. EGFP-labeled peptides that bound to BBMV proteins were detected in the blot using anti-EGFP antibodies. As this assay was only conducted once, the band intensities were not quantified. Note that binding of C6-EGFP is shown in vitro, although binding does not appear to occur in intact aphids

FIG. 4 shows relative in vivo binding of GBP3.1-EGFP and GBP3.1 alanine addition mutants to pea aphid gut. Arrow indicates location of protein bound to pea aphid gut. Control aphids fed with EGFP and diet only did not show any fluorescence in the gut. Non-binding peptide C6-EGFP showed binding to the pea aphid gut.

FIGS. 5A-5B illustrate analysis of purified CGAL1, CGAL2, CGAL3, CGAL4, CGAL5, CGAL7 and wild type Cyt2Aa. FIG. 5A: Purified proteins stained with Coomassie Brilliant Blue following separation by SDS-PAGE (12%), and FIG. 5B: purified proteins detected by western blot with anti-Cyt2Aa antibodies. Red box indicates position of purified proteins at ˜27 kDa (see below). Twenty μl protein was loaded on to the gel for protein staining in A, and 0.2 μl was loaded for western blot detection in FIG. 5B.

FIG. 6 shows activation of CGAL1, CGAL2, CGAL3, CGAL4, CGAL5, CGAL7 and wild type Cyt2Aa. Equal concentrations of each protein were loaded on to the gel. Proteins were detected by western blot with anti-Cyt2Aa antibodies. The protoxins at ˜27 kDa are smaller than the expected size of ˜31 kDa, suggesting that some proteolytic processing had occurred before in vitro activation. T: Trypsin; WT: wild type.

FIG. 7 compares binding of wild type and mutant Cyt2Aa to whole aphid BBMV. The amounts of whole aphid BBMV and test proteins used in the pull down assay were optimized. Cyt2Aa proteins that bound to BBMV proteins were detected in western blot with the anti-Cyt2Aa antibody. BBMV only, negative control; Positive control, Cyt2Aa protoxin.

FIG. 8 shows binding of wild type and mutant Cyt2Aa to whole aphid BBMV. The amounts of whole aphid BBMV and test proteins used in the pull down assay were optimized. Cyt2Aa proteins that bound to BBMV proteins were detected by western blot with the anti-Cyt2Aa antibody. BBMV only, negative control; Positive control, Cyt2Aa protoxin. A and B show 1 min and 3 min exposures, respectively.

FIG. 9 compares relative binding of wild type Cyt2Aa and Cyt2Aa—GBP3.1 addition mutants to whole aphid BBMV. No binding was detected for wild type Cyt2Aa. Band intensities in FIG. 11B were quantified using ImageJ software. Numbers on each bar indicate protein bound (ng) to whole aphid BBMV (10 μg starting material).

FIGS. 10A-10B compares binding of wild type and Cyt2Aa-GBP3.1 addition mutants to pea aphid gut aphid BBMV. The amounts of whole aphid BBMV and test proteins used in the pull down assay were optimized. Cyt2Aa proteins that bound to BBMV proteins were detected in western blot with the anti-Cyt2Aa antibody. BBMV only, negative control; Positive control, Cyt2Aa protoxin (FIG. 10 B). Band intensities in FIG. 10B were quantified using ImageJ software (FIG. 10A). Bars indicate concentration of toxins in ng bound to pea aphid gut BBMV (10 μg starting material).

FIG. 11 provides the results of a pea aphid feeding assay. The assay was run in duplicate with 10 aphids per treatment. Error bars indicate SEM. Bars with different letters are significantly different from each other (one way ANOVA).

FIGS. 12A-12F show the results of A. pisum and M. persicae feeding assays for CGAL1 (FIG. 10A), CGAL2 (FIG. 12B), CGAL3 (FIG. 12C), CGAL4 (FIG. 12D), CGAL5 (FIG. 12E), CGAL7 (FIG. 12F), Each assay was run in duplicate with 10 aphids per treatment. Error bars indicate SEM. LC50 values were estimated by probit analysis using PoloPlus statistical software and are presented in Table 2. One way ANOVA (p<0.05) was carried out for statistical analysis of differences between toxin fed aphids and the diet only controls. CGAL2, CGAL5 and CGAL7 did not appear to have significant toxicity against these aphids.

FIG. 13 shows the results of A. pisum and M. persicae bioassays of wild type Cyt2Aa. The assay was run in duplicate with 10 aphids per treatment. Error bars indicate SEM. LC₅₀ values could not be estimated from these data. One way ANOVA (p<0.05) was carried out between control and toxin fed aphids.

FIG. 14 illustrates comparative pea aphid feeding assay results for all Cyt2Aa-GBP3.1 addition mutants (CGAL1, CGAL2, CGAL3, CGAL4, CGAL5, CGAL7) and wild type Cyt2Aa. Each assay was run in duplicate with 10 aphids per treatment.

FIG. 15 illustrates comparative Myzus persicae feeding assay results for all Cyt2Aa-GBP3.1 addition mutants (CGAL1, CGAL2, CGAL3, CGAL4, CGAL5, CGAL7) and wild type Cyt2Aa. Each assay was run in duplicate with 10 aphids per treatment.

FIGS. 16A-16C show the effect of CGAL1 on the pea aphid guts membrane. (FIG. 16A) Cross section of the gut of a pea aphid fed on control diet. Intact and well-structured microvillar membranes (M) projecting into the gut lumen (L) are apparent. (FIG. 16B) Cross section of the gut of a pea aphid fed for approximately 72 hr on CGAL1 (75 μg/ml). Arrows indicate damaged M. (FIG. 16C) Cross section of the gut of a pea aphid fed on wild type Cyt2Aa (75 μg/ml). Images are taken at 100×. Details of selected regions of FIGS. 16A, B and C are shown above each image.

FIG. 17 shows the destruction of pea gut aphid membrane by CGAL1. Transmission electron micrographs show the intact apical surface of the gut membrane and well-structured microvillar membranes (M) projecting into the gut lumen (L) in aphids fed on control diet (Control). In contrast, the guts of aphids fed on CGAL1 were severely damaged (CGAL1), and minor damage was observed in aphids fed on Cyt2Aa (WT Cyt2Aa). Second instar pea aphids were fed on a single concentration (100 μg/ml) of CGAL1 or Cyt2Aa in complete artificial diet by membrane feeding. Control aphids were fed on diet alone. The assay was set up in triplicate with ten aphids per replicate in a growth chamber at 24° C. with an 18:6 light:dark photoperiod. After a period of approximately 72 hr, aphids from all groups were collected. The rear abdomen was cut and aphids were fixed in a fixative solution containing embedded resin and sent to the Microscopy and Nanolmaging Facility at Iowa State University for processing. One fixed whole aphid from each treatment group was processed with vertical (head to tail) microtome sections prepared and slides observed under an electron microscope.

FIGS. 18A-18B illustrate analysis of purified CGSL1, CGSL2, CGSL3, CGSL4, CGSL5, CGSL7 and wild type Cyt2Aa. FIG. 18A: Purified proteins stained with Coomassie Brilliant Blue following separation by SDS-PAGE (12%), and FIG. 18B: purified proteins detected by western blot with anti-Cyt2Aa antibodies. Red box indicates position of purified proteins at ˜27 kDa (see below). Twenty μl protein was loaded on to the gel for protein staining in FIG. 18 A, and 0.2 μl was loaded for western blot detection in FIG. 18B.

FIG. 19 shows activation of CGSL1, CGSL2, CGSL3, CGSL4, CGSL5, and CGSL7. Equal concentrations of each protein were loaded on to the gel. Proteins were detected by western blot with anti-Cyt2Aa antibodies. The protoxins at ˜27 kDa are smaller than the expected size of ˜31 kDa, suggesting that some proteolytic processing had occurred before in vitro activation. T: Trypsin.

FIG. 20 illustrates comparative pea aphid feeding assay results for all Cyt2Aa-GBP3.1 substitution mutants (CGSL1, CGSL2, CGSL4, CGSL5, CGSL7) and wild type Cyt2Aa. Each assay was run in duplicate with 10 aphids per treatment. LC50 values were estimated by probit analysis using PoloPlus statistical software and are presented in Table 3. CGSL3 was highly unstable hence was not included in the feeding assays. CGSL2, CGSL5 and CGSL7 did not appear to have significant toxicity against these aphids.

FIG. 21 illustrates the strategy for expression of a chimeric toxin comprising GBP3.1 or GBP3.1-derived peptide, peptide multimer or thereof. The target coding sequences are cloned into multiple cloning site 2 (MCS2). P35s: promoter; Ω: expression enhancer; Tnos: Nos terminator; Bar: Bar gene, coding the phosphinothricin acetyltransferase (PAT) that confers resistance to the herbicide phosphinothricin (PPT) for selection of transformed plants. RB and LB: right and left T-DNA borders for inserting the expression cassette into the plant genome.

### DETAILED DESCRIPTION

The terms “fusion protein” or chimeric protein or chimeric toxin are used herein to describe a protein comprising portions from different sources (not both parts of the same naturally occurring polypeptide chain). Optionally, a linker region may be included to facilitate folding of the domains (portions) into their natural conformations by reducing steric hindrance between those domains. Such a fusion protein may have an additional domain, for example a tag sequence to facilitate purification of the fusion protein. A tag can be any of a number of known tags widely known and available to the art (Streptavidin-binding, glutathione binding, polyhistidine, flagellar antigen and others).

A chimeric insecticidal toxin is one in which there is a peptide portion which mediates binding of the chimeric toxin to an insect gut membrane and thus allows toxicity in an insect in which the insecticidal toxin without that peptide portion is not active or has very little activity. A specific example is the Bacillus thuringiensis Cyt2A protein, for which coding and amino acid sequences are provided herein (see SEQ ID NOs:25 and 26). Other examples include the Bacillus thuringiensis crystal proteins, including Cry4Aa, into which a gut binding peptide as described herein (of amino acid sequence set forth in SEQ ID NO:1-20 or matching consensus sequence SEQ ID NO:21).

A Bacillus thuringiensis insecticidal toxin or chimeric insecticidal toxin (protein) is one which kills at least one insect, in at least one stage of the development of that insect (larva, adult, for example).

In the present context, the term “peptide” does not encompass the full-length protein from which the peptide's sequence was derived. However, in the context of the present disclosure, the term peptide encompasses a single peptide of 3 to 14 amino acids as well as an oligopeptide or polypeptide made up of repeats of identical or non-identical amino acid sequences, each of which fits the consensus sequence of SEQ ID NO:29 or core amino acid sequences shared by various segments of the pea enation mosaic virus (PEMV) coat protein or other virus coat proteins or other peptides or proteins which mediate binding to an insect midgut or hindgut receptor. Advantageously, the peptide mediates binding of a chimeric toxin comprising the peptide to an insect gut so that the insect is killed by the chimeric toxin.

The term “plant” is intended to include a whole plant, any part of a plant, a seed, a fruit, propagules and progeny of a plant.

The term “propagule” means any part of a plant that may be used in reproduction or propagation, either sexual or asexual, including seeds and cuttings.

As used herein, a target insect is an insect to be killed or inhibited in feeding by a chimeric insecticidal protein as described herein. Aphids, as well as thrips, planthoppers and other sap-sucking insects are of particular importance in agriculture and horticulture.

Crop plants and agricultural plants are those of economic importance for human or animal food production or for animal fodder production and can include grains, fruits and vegetables as well as grasses. Horticultural plants include those for turfgrass, windbreaks and landscaping and include ornamental plants such as flowers, shrubs, vines and the like, and especially members of the Rosaeceae.

On the basis that crystal (Cry) toxins are more acceptable than the cytolytic (Cyt) toxins for field use due to their specificity and that Cry4Aa has a low level of toxicity against the pea aphid, this toxin provides an ideal candidate for improvement of aphid toxicity to a level sufficient for use in aphid resistant transgenic plants. However, the physiological factors that account for the relatively low aphid toxicity of Cry4Aa, including whether the toxin interacts with the putative receptor proteins, are unknown.

The addition of GBP3.1 to Cyt2Aa was highly effective in producing an aphicidal toxin, and addition of this sequence or a related gut binding peptide to a Cry toxin has the same effect. While the interaction of cytolytic toxins is lipid-based, Bt Cry toxins interact with specific insect gut receptors such as aminopeptidases N, cadherin and alkaline phosphatase (ALP), and the aphid gut component bound by GBP3.1 has yet to be identified. Although the peptide GBP3.1 binds to the guts of several aphid species including the soybean aphid, Aphis glycines, it is believed that Cry4Aa modified with this gut binding peptide is effective for management of multiple aphid pests. Having identified a novel strategy that has potential for sustainable aphid management, it is useful to apply this strategy to toxins that are acceptable for use in transgenic plants.

A phage display library for peptides that bind to the gut of the pea aphid, Acyrthosiphon pisum (Harris) was screened, and an aphid gut binding peptide, GBP3.1, was isolated. Addition of this peptide and certain variants thereof to the model toxin, Cyt2Aa resulted in significant aphicidal activity of the modified toxin, as compared with the naturally occurring toxin protein. This same approach is applicable to gut binding peptides for other insect species where biological insect control is needed and where any toxin such as a Bt toxin is not naturally sufficiently active to allow control of one or more target insects.

Results described herein exemplify an embodiment wherein the peptide is GBP3.1. SEQ ID NO:1 provides the sequence of the GBP3.1-peptide having N- and C-terminal alanine residues. However, the N- and C-terminal alanine residues are not required for functional activity of this peptide. The method described herein for identifying GBP3.1 from a phage library and demonstrating its activity are readily applied to isolating and characterizing other peptides that mediate binding to the guts of insects other than A. pisum, including, but not limited to, insects within the order Hemiptera, members of which include aphids and planthoppers, white flies, and the order Thysanoptera, which includes thrips. Particularly relevant insects are aphids, species of which include Rhopalosiphum padi, Sitobion avenae, Microsiphum avenae, Schizaphis graminum, and Acyrthosiphon pisum and Myzus persicae. Other modifications include certain substitution variants of the GBP3.1. In chimeric gut binding peptide-toxin proteins, toxicity is correlated with the extent of binding to the insect gut membrane.

Also provided are plants which have been genetically engineered to contain within their genomes and express a chimeric aphicidal or other chimeric insecticidal protein as described herein. An insecticidal protein can be made aphicidal by incorporating a peptide portion as described herein which binds to the cell membrane of the aphid gut, for example, midgut. Plants susceptible to attack by aphids of particular relevance include, without limitation, essentially all agricultural and food plants such as sugar beets, legumes, soybean (Glycine max), Chenopodium album, Chenopodium amaranticolor, Chenopodium quinoa, Cicer arietinum, Lathyrus odoratus, Lens culinaris, Lespedeza stipulacea, Lupinus albus, Lupinus angustifolius, Medicago Arabica, Medicago sativa, Melilotus albus, Nicotiana clevelandii, Phaseolus vulgaris, Pisum sativum, Trifolium hybridum, Trifolium incarnatum, Trifolium repens, Trifolium subterraneum, Vicia faba, Vicia sativa and Vicia villosa, stone fruits, pears, peaches, grapes, berries, apples, wheat and other grains, tobacco, vegetables including, without limitation, tomato, potato, and essentially all horticultural and ornamental plants including roses (Rosa species), among others. Expression of a chimeric aphicidal protein or other chimeric insecticidal protein as described herein in these plants decreases plant damage due to aphids or other target insect. It is understood when the target insect is a sap-sucking insect, the chimeric insecticidal or aphicidal protein is expressed in the phloem fluids of that plant. For leaf-chewing insects and for certain insects, including hemipteran insects that feed on xylem, the chimeric insecticidal protein is expressed in the leaf and/or stems. The art knows constitutive promoters which result in expression in essentially all plant tissues and promoters that are preferentially expressed in phloem, leaf, or root tissue or which are expressed in response to environmental signals such as light.

Light-regulated promoters include, for example, those of the well-known genes encoding small subunit of riboulose-5-bisphosphate carboxylase of soybean, chlorophyll a binding protein, among others; see, e.g., U.S. Pat. Nos. 5,639,952; 5,656,496 and 5,750,385, among others.

For control of hemipteran insects that feed on phloem, phloem cell-specific or phloem-preferred promoters can be used to express a gut binding chimeric insecticidal toxin of interest in the phloem of transgenic plants. Phloem specific promoters known to the art include, without limitation, the CmGAS1 promoter described in U.S. Pat. No. 6,613,960; the Agrobacterium rhizogenes RoIC promoter (Graham et al., 1993); and the pumpkin PP2 promoter (Dinant et al. 2004).

Phloem-limited viruses have been described, including but not limited to the rice tungro virus (Bhattacharyya-Pakrasi et al. 1993. Plant J. 4, 71-79) and the commelina yellow mottle virus (Medberry et al. 1992. Plant Cell 4:185-192) also contain useful promoters that are active in vascular tissues. Others are described in U.S. Pat. No. 5,494,007; U.S. Pat. Nos. 5,824,857; 5,789,656; 6,613,960; and US Patent Publication 20100064394, and Guo et al. 2004; Graham et al. 1997, among others.

Examples of additional useful phloem specific promoters include, but are not limited to, PP2-type gene promoters (U.S. Pat. No. 5,495,007), sucrose synthase promoters (Yang and Russell, 1990. Proc. Natl. Acad. Sci. USA 87:4144-4148, 1990), glutamine synthetase promoters (Edwards et al. 1990. Proc. Natl. Acad. Sci. USA 87:3459-3463, 1990), and phloem-specific plasma membrane H+-ATPase promoters (DeWitt et al. 1991. Plant J. 1, 121-128, 1991), prunasin hydrolase promoters (U.S. Pat. No. 6,797,859), and a rice sucrose transporter (U.S. Pat. No. 7,186,821). For control of hemipteran pests that feed on xylem tissue, a variety of promoters that are active in xylem tissue including, but not limited to, protoxylem or metaxylem can be used.

One broad class of useful promoters is referred to as “constitutive” promoters in that they are active in most plant organs throughout plant development. For example, the promoter can be a viral promoter such as a CaMV35S or FMV35S promoter. The CaMV35S and FMV35S promoters are active in a variety of transformed plant tissues and most plant organs (e.g., callus, leaf, seed and root). Enhanced or duplicated versions of the CaMV35S and FMV35S promoters are particularly useful in expression of a gene of interest throughout a transgenic plant (see U.S. Pat. No. 5,378,619, incorporated herein by reference). Other useful nopaline synthase (NOS) and octopine synthase (OCS) promoters (which are carried on tumor-inducing plasmids of A. tumefaciens), the cauliflower mosaic virus (CaMV) 19S or 35S promoters, a maize ubiquitin promoter (U.S. Pat. No. 5,510,474), the rice Act1 promoter and the Figwort Mosaic Virus (FMV) 35S promoter (see e.g., U.S. Pat. No. 5,463,175; incorporated herein by reference). It is understood that this group of exemplary promoters is non-limiting and that one skilled in the art could employ other promoters that are not explicitly cited herein. Promoters that are active in certain plant tissues (i.e., tissue specific promoters) can also be used to drive expression of chimeric insecticidal toxin proteins as described herein. Since certain hemipteran insect pests are “piercing/sucking” insects that typically feed by inserting their proboscis into the vascular tissue of host plants, promoters that direct expression of insect inhibitory agents in the vascular tissue of the transgenic plants are particularly useful in the expression of a chimeric insecticidal toxin as described herein. Various Caulimovirus promoters, including but not limited to the CaMV35S, CaMV19S, FMV35S promoters and enhanced or duplicated versions thereof, typically deliver high levels of expression in vascular tissues and are thus useful for expression of a chimeric insecticidal toxin protein as described herein.

Promoters active in xylem tissue include, but are not limited to, promoters associated with phenylpropanoid biosynthetic pathways, such as the phenylalanine ammonia-lyase (PAL) promoters, cinnamate 4-hydroxylase (C4H) promoters, coumarate 3-hydroxylase promoters, O-methyl transferase (OMT) promoters, 4-coumarate:CoA ligase (4CL) promoters (U.S. Pat. No. 6,831,208), cinnamoyl-CoA reductase (CCR) promoters and cinnamyl alcohol dehydrogenase (CAD) promoters.

Further provided are methods for making a chimeric insecticidal protein, said method comprising the steps of isolating a peptide which binds to the gut of the target insect, inserting the gut-binding peptide into an insecticidal protein to product a chimeric insecticidal protein and verifying insecticidal activity in the target insect.

Also provided herein are methods of control of a target insect comprising providing a transgenic plant which contains in its genome and expresses a chimeric aphicidal or other chimeric insecticidal protein, wherein that chimeric protein comprises a gut-binding peptide portion which is specific to the target insect and which is not in nature associated with that insecticidal protein. It is understood when the target insect is a sap-sucking insect, the chimeric insecticidal or aphicidal protein is expressed in the phloem fluids of that plant. For leaf-chewing insects and certain insects that feed on xylem, the chimeric insecticidal protein is expressed in the leaf and/or stem. The art knows constitutive promoters which result in expression in essentially all plant tissues and promoters that are preferentially expressed in phloem, leaf, or root tissue or which are expressed in response to environmental signals such as light.

Further provided are methods for reducing spread of a plant pathogen, for example, a plant pathogenic virus, carried by a target insect, comprising providing a transgenic plant which contains in its genome and expresses a chimeric aphicidal or other chimeric insecticidal protein, wherein that chimeric protein comprises a gut-binding peptide portion which is specific to the target insect and which is not in nature associated with that insecticidal protein. The gut-binding peptide which is part of the chimeric toxin described herein competes with viruses for binding sites on the insect gut membrane. It is understood when the target insect is a sap-sucking insect, the chimeric insecticidal or aphicidal protein is expressed in the phloem fluids of that plant. For leaf-chewing insects, the chimeric insecticidal protein is expressed in the leaf. The art knows constitutive promoters which result in expression in essentially all plant tissues and promoters that are preferentially expressed in phloem, leaf, or root tissue or which are expressed in response to environmental signals such as light.

### EXAMPLES

Aphids. The pea aphid (Acyrthosiphon pisum) was used for these experiments. A. pisum were reared on pea (22-24° C., L; D 12:12 hrs). The phage display library (derived from phage f88.4) was provided by Dr. Jamie Scott, Simon Fraser University, Canada (Smith, G. P. and Scott, J. K. (1993) Methods in Enzymology 217:228-257). Phage were cultured in Escherichia coli K-91. The library was cultured, amplified, purified and titered using standard procedures.

Membrane feeding of aphids with phage. To optimize feeding of aphids on solutions of phage, aphids were held without food overnight at 4° C. and then fed through Parafilm membranes on phage in a 25% sucrose and 10-15% glycerol solution. This protocol improves feeding efficiency over previous methods.

Isolation of aphid guts. A wax-embedding method developed to trap aphids for isolation of aphid hemolymph (blood) (Liu, S. et al. (2006) was modified for isolation of aphid guts. Aphids were embedded in black wax, and covered with phosphate buffered saline (PBS) buffer prior to dissection using a binocular microscope. The black background facilitated visualization and identification of the gut. Guts were isolated and transferred to eppendorf tubes.

Elution of bound phage. Because of the small size of aphid guts, it was not possible to cut them open to wash out unbound phage, and then elute bound phage as described previously for mosquitoes (Ghosh, A. K. et al. (2001) Proc Natl. Acad. Sci. USA 98:13278-13281; Jacobs-Lorena, M. (2003) J Vector Borne Dis. 40:73-77; James, A. A. (2003) J. Experimental Biology 206:3817-3821). Aphid guts were cut into small pieces with a sharp needle, and gently homogenized in a 1.5 ml tube. After two rounds of washing, bound phage were eluted by adding elution buffer. Eluted phage were titrated to estimate the number of phage bound to the aphid gut. Eluted phage were unstable in elution buffer and were immediately amplified for use in the next round of bio-panning.

Three rounds of bio-panning were conducted to select for phage that bound to the aphid gut epithelium. The whole procedure was replicated twice. A phage displaying the sequence given in SEQ ID NO:1 was isolated and named PhD3.1.

Isolation of PhD3.1 by bio-panning. Bio-panning was conducted by feeding aphids with phage, isolation of aphid guts, washing for removal of unbound phage, and elution of phage bound to the gut epithelium. Eluted phage were amplified and used for the next round of bio-panning. After each round of bio-panning between 400 and 1000 phage were recovered from the aphid gut epithelium. After the third round of bio-panning, eluted phage were isolated and the DNA from each of 14 phage was extracted. The DNA sequences encoding the peptides displayed by each phage were determined. All 14 of the eluted phage, isolated after the third round of selection, encoded the same peptide sequence as PhD3.1 (SEQ ID NO:9). Replication of the whole experiment gave the same result. To confirm that the phage display library encoded diverse peptide sequences, 10 phage from the library along with 10 phage from each of the first and second rounds of eluted phage were sequenced. All 10 phage sequenced from the original library had different encoded peptide sequences. Of phage eluted from the first and second rounds, zero and four had the same sequence as PhD3.1, respectively. These results indicate that the peptide sequence (SEQ ID NO:9) displayed by PhD3.1 was selected from a diverse phage population during the three rounds of bio-panning.

This same panning strategy is employed to isolate and identify gut binding peptides for other target insects, for which a chimeric Bt-based insect control strategy is needed, i.e. target insects which are not sufficiently sensitive to the naturally occurring Bt toxin for its use. The gut binding peptides are expressed as part of a Bt Cyt2Aa or Cry4A protein through molecular biological methods, especially in a plant to be protected from that insect.

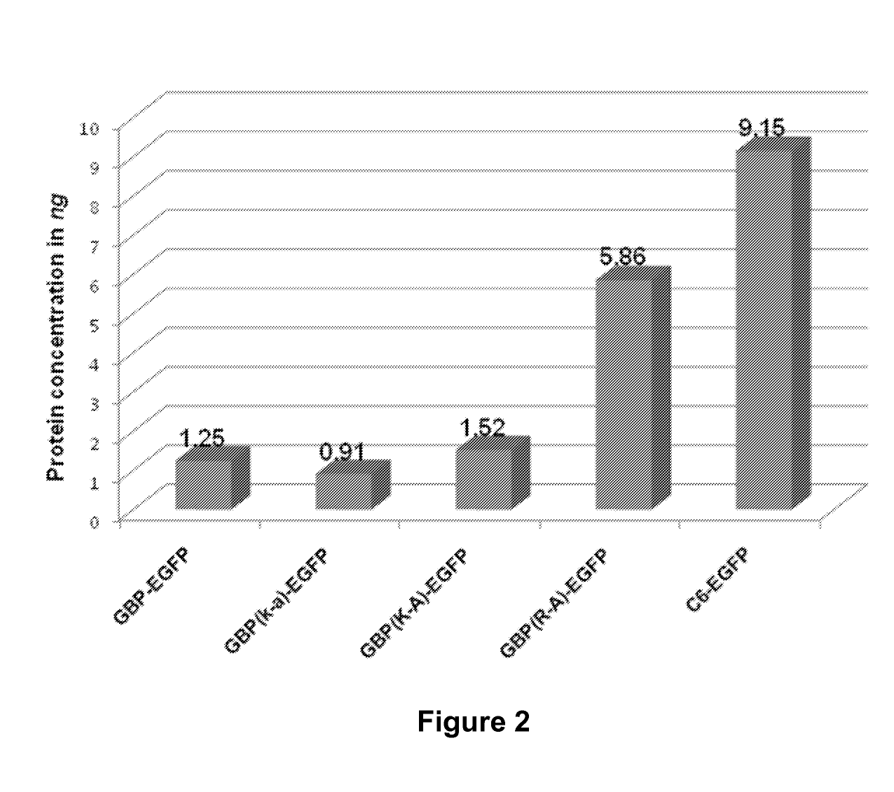

Mapping the PhD3.1 sequence to a potential epitope on PEMV and other luteovirus coat proteins. All nine CP fragments shown in FIG. 2 of U.S. Pat. No. 7,547,677 may recognize receptors on the surface of the aphid gut epithelium, thereby mediating uptake of the virus into the aphid hemocoel. However, an epitope in the CP of Potato leafroll virus (PLRV), which is recognized by a monoclonal antibody has been characterized (Torrance, L. 1992. Virology 191(1):485-489; Terradot et al. 2001. Virology 286, 72-82). This epitope H is Asp Ser Ser Glu Asp Gln (SEQ ID NO:23) was predicted to be on the surface of PLRV. There is a similar motif between amino acid positions 65-78 of CP: Gly Pro Ser Ser Asp Cys Gln (SEQ ID NO:24). The core epitope amino acids in PLRV are Ser Ser Glu Asp Gln (SEQ ID NO:25), compared to Ser Ser Asp Cys Gln (SEQ ID NO:26) in PEMV. Peptide portions corresponding to SEQ ID NOs:40-41 are useful for mediating gut binding of a chimeric insecticidal toxin containing same in an aphid or other insect attacking potato and therefore allowing for use in insect control, especially when that chimeric insecticidal toxin is expressed in phloem of transgenic potato.

These results suggest that the peptide expressed and displayed by PhD3.1 can bind to receptors that mediate PEMV uptake into the aphid hemocoel. This peptide can also be effective for blocking uptake of other plant viruses such as PLRV in other aphid or sap-sucking insect vectors that have similar gut receptor sites to that of the aphid.

Purification of 6×His-tagged proteins by Ni-NTA column chromatography. Recombinantly expressed His-tagged proteins were purified using Ni-NTA (nickel-nitrilotriacetate) agarose resin (Qiagen) according to the manufacturer's instructions. The purification was under native conditions, and a batch purification method was used. All the steps for the protein purification were performed either on ice or at 4° C.

The harvested bacterial pellets were resuspended in 5 ml of lysis buffer and sonicated on ice. The sonicated resuspensions were transferred to 1.5 ml tubes and centrifuged (rcf 1000×g) in a bench-top centrifuge for 5 min at 4° C. Supernatants were mixed with 1 ml of Ni-NTA resin in a 15 ml centrifuge tube (Fisher Scientific) and incubated by rotating at 4° C. for 2 hrs before being loaded into a 1 ml Polypropylene Column (Qiagen). The column was washed with 4×5 ml washing buffer and eluted with 3 ml of elution buffer. Elution was collected in 500 μl fractions. 20 μl of each fraction was checked by separation in a 12% SDS-polyacrylamide gel.

Fractions containing the fusion proteins were stored at −80° C. As needed, the purified proteins were concentrated by YM-3 Centricon Centrifugal Filter Devices (Amicon, Beverly, Mass.) and dialyzed in Side-A-Lyzer Dialysis Cassettes (0.1-0.5 ml capacity and 3,500 molecular weight cutoff (MWCO)) (Pierce Chemical Co., Rockford, Ill.) with PBS buffer.

The use of the EFGP allowed visualization of fusion proteins in the aphids upon exposure to ultraviolet light.

GBP3.1-EGFP and the control construct C6-EGFP were expressed in E. coli with 6×His tags and purified.

EGFP, GBP3.1-EGFP or C6-EGFP was fed to pea aphids by membrane feeding (Chay et al., 1996, supra). In contrast to the two control treatments where only background fluorescence was apparent, areas of fluorescence were seen in aphids that ingested GBP3.1-EGFP. Aphid guts were dissected and observed under normal light (top panel) and under UV light to observe fluorescence. Fluorescence was only detected in aphids fed with GBP3.1-EGFP.

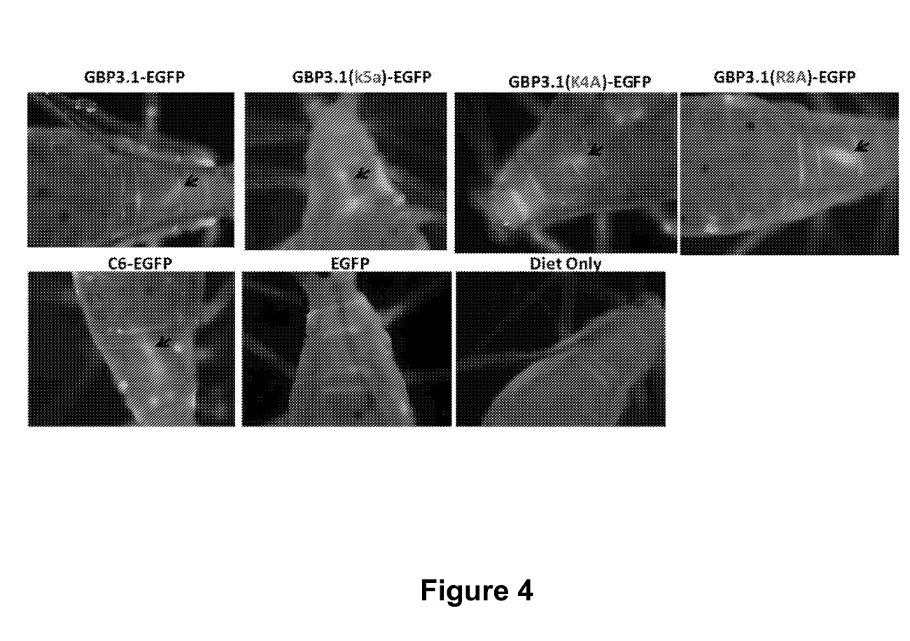

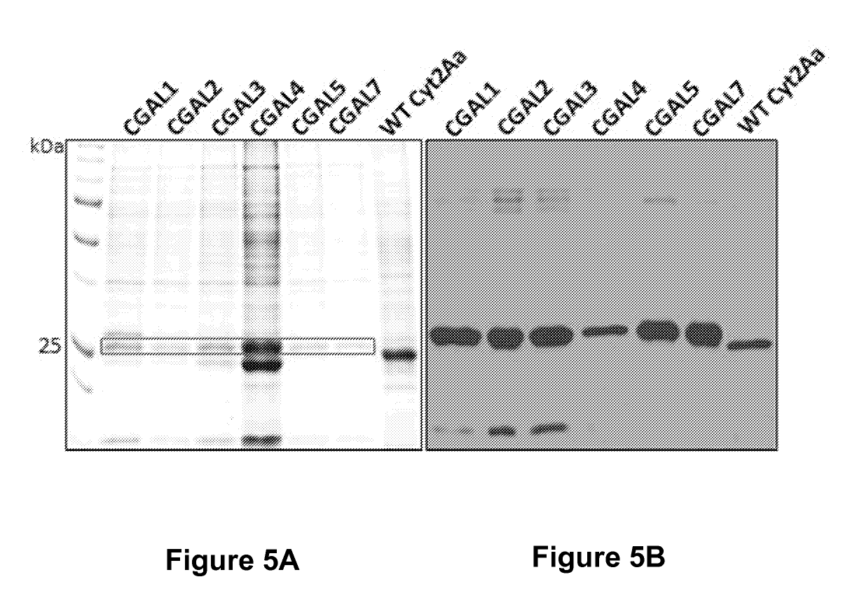

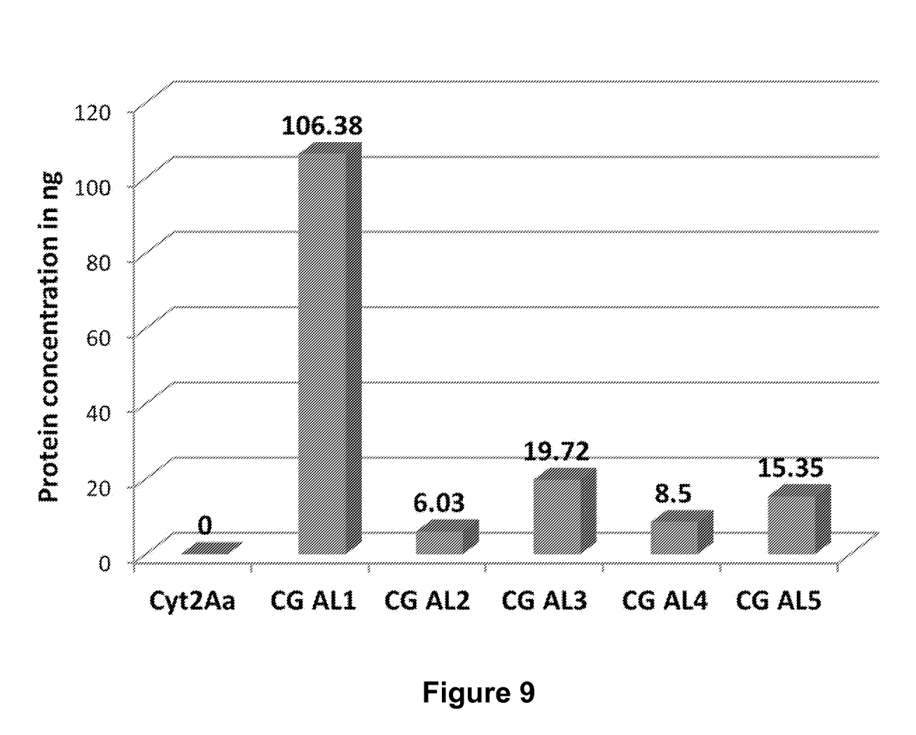

Fluorescence was localized to the gut of aphids that fed on GBP3.1-EGFP. These results indicate that following feeding the majority of GBP3.1-EGFP either bound to the gut surface or entered the gut cells, but did not appear to enter the hemocoel (body cavity) of the aphid. The results are depicted in FIG. 4, FIG. 9 and Liu et al., 2010.

Pull down assay. The method described by Perez et al. (2005) (PNAS 102:18303-1808) was used for a pull down assay. Briefly, 10 μg whole pea aphid BBMV was incubated with 50 nM (125 ng) of GBP3.1-EGFP, Alanine mutant, C6-EGFP or EGFP in 100 μl binding buffer (1×PBS pH 7.4, 0.1% BSA, 0.1% Tween20) for 1 hr at room temperature, and centrifuged at 14,000 rpm for 15 min at 4° C. The pelleted BBMV were washed three times with 500 μl binding buffer. The final BBMV pellet was resuspended in 10 μl 1×SDS sample buffer, boiled for 5 min, and proteins separated by 12% SDS-PAGE. Proteins were then detected by western blotting using commercially available anti-GFP antibodies. To quantify the relative binding to pea aphid BBMV, ImageJ software was used to measure the intensities of each band in a scanned image of the blot, which were then compared with the intensities of known amounts of protein. The experiment was conducted twice, with the ImageJ analysis conducted for one blot.

BBMV of other target insects can be used in place of the pea aphid BBMV to identify gut binding peptide useful for making a chimeric insecticidal toxin active in that target insect.

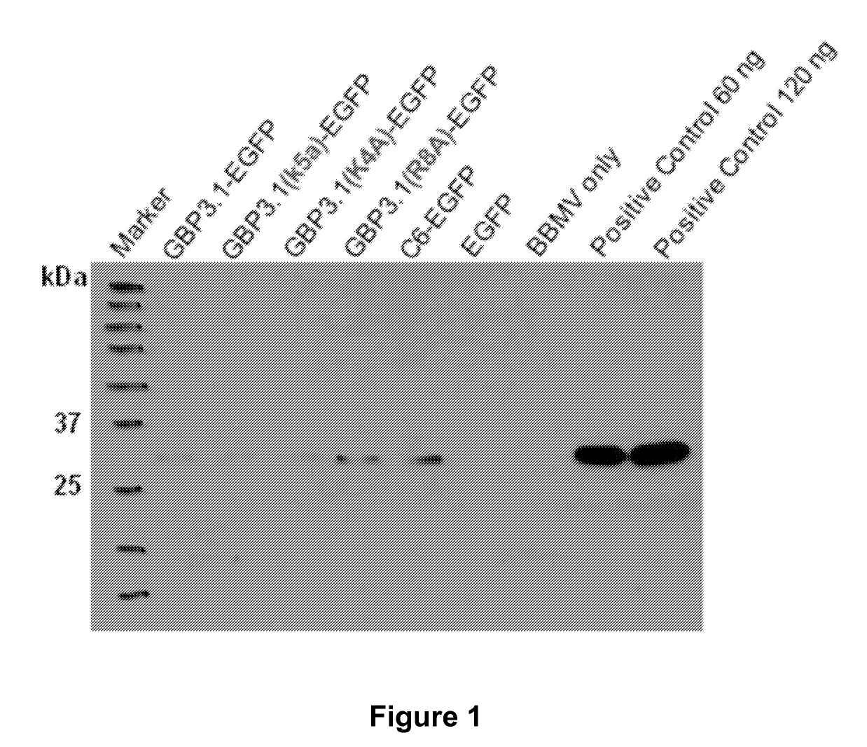

Relative binding of GBP3.1 and alanine mutants to pea aphid BBMV. None of the three mutations made in GBP3.1 completely abolished binding to pea aphid BBMV (FIG. 1 and FIG. 4). A band of ˜29 kDa was detected in all GBP3.1 and mutant pull down assay reactions, while no bands were detected in the EGFP only, and BBMV only control reactions. C6-EGFP (the in vivo control non-binding peptide) showed greater binding than any of the other peptides tested under these in vitro conditions. The band from the GBP3.1(R8A)EGFP mutant appeared more intense than that of GBP3.1-EGFP, indicating improved binding to pea aphid gut proteins. Bands from reactions with GBP3.1(K5A)-EGFP and GBP3.1(K4A)-EGFP were similar in intensity to that of GBP3.1-EGFP. The blots generated for the two experimental replicates were similar.

The ImageJ analysis indicated an almost five-fold increase in the pea aphid BBMV binding efficiency of GBP3.1(R4A)-EGFP, while the other GBP3.1 mutants showed similar binding efficiency to GBP3.1 (FIG. 2). C6-EGFP showed ˜7.5-fold more binding to BBMV than GBP3.1-EGFP.

In summary, Alanine mutants of GBP3.1 varied from GBP3.1 in their pea aphid BBMV binding properties. Substitutions of Ala for Lys4 or Lys5 had little effect on binding, while substitution of Ala for Arg8 improved BBMV binding by almost five-fold relative to GBP3.1.

Construction of novel Cyt2Aa proteins with aphid toxicity. The Example describes construction of novel, aphicidally active Cyt2Aa by introducing an aphid gut binding peptide into the toxin. Constructs for addition to, or substitution of, the Cyt2Aa loops with the 12 amino acid GBP3.1 or variants thereof have been made. Sequences are provided herein below.

Additional mutants were first selected to test for pea aphid toxicity and then subjected to functional characterization, especially for the two addition mutants Cyt2Aa-His-Ek-GBP-AL1 (CGAL1) and Cyt2Aa-His-Ek-GBP-AL3 (CGAL3), and additional chimeric proteins were also tested for gut binding and toxicity.

Expression, purification and activation of CGAL1, CGAL3 and other engineered proteins. A single colony of recombinant bacteria expressing the chimeric fusion protein of interest from a freshly streaked plate was inoculated into 2 ml LB+Carbanicillin and incubated overnight at 37° C. The following day, 50 μl of the overnight culture was inoculated into 50 ml fresh LB medium and incubated at 37° C. until the optical density at 600 nm (0D₆₀₀) of the culture reached approximately 0.5. IPTG was added to a final concentration of 1 mM to induce recombinant protein expression in the culture. The culture was incubated at 37° C. overnight after induction. Cells were pelleted by centrifugation at 3500 rpm for 25 min at 4° C., resuspended in 10 ml of 50 mM Tris-HCl (pH 7.5) containing 10 mM KCl and 0.01% Triton X100, 10 mM EDTA and 1 mM PMSF and sonicated on ice 10 times with a 1 min ON/OFF cycle at level 6. The cell lysate was spun at 10,000 rpm for 10 min at 4° C., pelleted inclusion bodies were washed three times with chilled water and the final pellet was solubilized in 1 ml of 50 mM Na₂CO₃ pH 10.5 buffer at 37° C. for 1 hr. Solubilized toxin was obtained by centrifugation at 10,000 rpm for 10 min at 4° C. The clear supernatant containing recombinant protein was transferred to a fresh tube and checked by SDS-PAGE for purity. Western blot analysis with anti-Cyt2Aa antibodies was also conducted.

For activation of CGAL1 and CGAL3 or other Bt-derived protein of interest, trypsin was used at a final concentration equal to 5% of the toxin concentration. A trypsin concentration higher than 1% was needed due to the presence of contaminating proteins in the final preparations of CGAL1 and CGAL3. The CGAL1 and CGAL3 preparations were adjusted to pH 7.5 before the activation reaction, as trypsin has optimal activity and specificity around pH 7.5. Reactions were incubated at 37° C. for 45 min. Small aliquots of activated CGAL1 and CGAL3 were boiled in 1×SDS sample buffer for 5 min and analyzed by 12% SDS-PAGE and western blot using anti-Cyt2Aa antibodies. After complete activation of CGAL1 and CGAL3, residual trypsin from the activation reaction was removed by Benzamidine Sepharose B affinity chromatography (GE Healthcare). Complete removal of trypsin from activated CGAL1 and CGAL3 was confirmed in an trypsin activity assay using BApNA as a substrate. The remaining active Cyt2Aa preparation was stored at −20° C. until further use.

Whole aphid BBMV binding assay. Whole pea aphid BBMV (20 μg) was incubated with 50 nM active Cyt2Aa, active CGAL1 or active CGAL3 (or other engineered protein of interest) in 100 μl binding buffer (1×PBS pH 7.4, 0.1% BSA, 0.1% Tween20) for 1 hr at room temperature, centrifuged at 14,000 rpm for 15 min at 4° C. The pelleted BBMV were washed three times with 500 μl of binding buffer. The final BBMV pellet was resuspended in 10 μl 1×SDS sample buffer, boiled for 5 min, separated by 12% SDS-PAGE and bound proteins detected by western blot using anti-Cyt2Aa antibodies.

Mosquito and pea aphid feeding assays. Early third instar Aedes aegypti larvae were used in feeding assays to confirm the toxicity of purified recombinant Cyt2Aa, CGAL1, CGAL3, and CGAL4 or other engineered protein of interest. Assays were set up in 24-well cell culture plates with 2 ml of protein solution in distilled water in each well. Toxin dilutions ranged from 12.5 μg/ml to 0.097 μg/ml in serial two-fold dilutions. Eight different groups (control, pro-Cyt2Aa, active Cyt2Aa, pro-CGAL1, active CGAL1, pro-CGAL3, active CGAL3, CGAL4 and vector protein control) were set up in duplicate with 10-15 larvae per well. The protein preparation was neutralized to pH 7.5 with 1 N HCl before being used in the feeding assay. Plates were incubated in an incubator at 30° C. with 75% humidity and an 18:6 light:dark photoperiod. Mortality of larvae was recorded daily, and the assay was run for 50 hr.

For the pea aphid feeding assay, two protein concentrations (100 μg/ml and 50 μg/ml) or five doses up to a maximum of 150 μg/ml (see tables herein) of the pro- and active forms of Cyt2Aa and chimeric toxins were used. In addition to the diet only control, control treatments of vector protein only, as well as BSA were set up to control for contaminating proteins and high protein concentration in the diet. Complete artificial liquid diet (Febvay et al. 1988) and second instar pea aphids were used in the assay. Treatments were set up in duplicate. The protein preparation was neutralized to pH 7.5 with 1 N HCl before use in the feeding assay. The assay was set up in a growth chamber at 24° C. with an 18:6 light:dark photoperiod. Mortality was recorded every 24 hr and diet was replaced every third day. The feeding assay was continued for 7 days. Expression of Cyt2Aa-GBP3.1 addition or substitution mutants in E. coli BL21 DE3 PLysE. Previously prepared Cyt2Aa-GBP3.1 addition/pGEMTeasy mutants were transformed into E. coli BL21 DE3 PLysE and screened for expression. Small scale analysis of the expression of four Cyt2Aa-GBP3.1 addition mutants (AL2, AL4, AL5 and AL7) in pGEM-Teasy was carried out. Four clones of each construct were selected for this screen. Individual colonies from freshly streaked plates were inoculated into 1 ml fresh LB medium containing 50 μg/ml Carbanicillin and incubated on a shaker overnight at 37° C. Four μl of the overnight culture was inoculated into 4 ml fresh LB medium and incubated at 37° C. until the OD₆₀₀ reached about 0.5. IPTG was added to a final concentration of 1 mM to induce recombinant protein expression. The culture was incubated at 37° C. overnight (or about 16 hr) after induction. Wild type Cyt2Aa was included in the experiment to compare expression levels with those of the recombinant proteins. Cells from the 20 μl culture were pelleted by centrifugation at 3,500 rpm for 10 min at 4° C., resuspended in 10 ml of 50 mM Tris-HCl pH 7.5 containing 10 mM KCl. 1×SDS sample buffer was added and the sample boiled for 10 min to lyse the cells, solubilize and denature the recombinant protein. Proteins were separated by 12% SDS-PAGE and recombinant proteins detected by western blot using anti-Cyt2Aa antibodies.

### Results

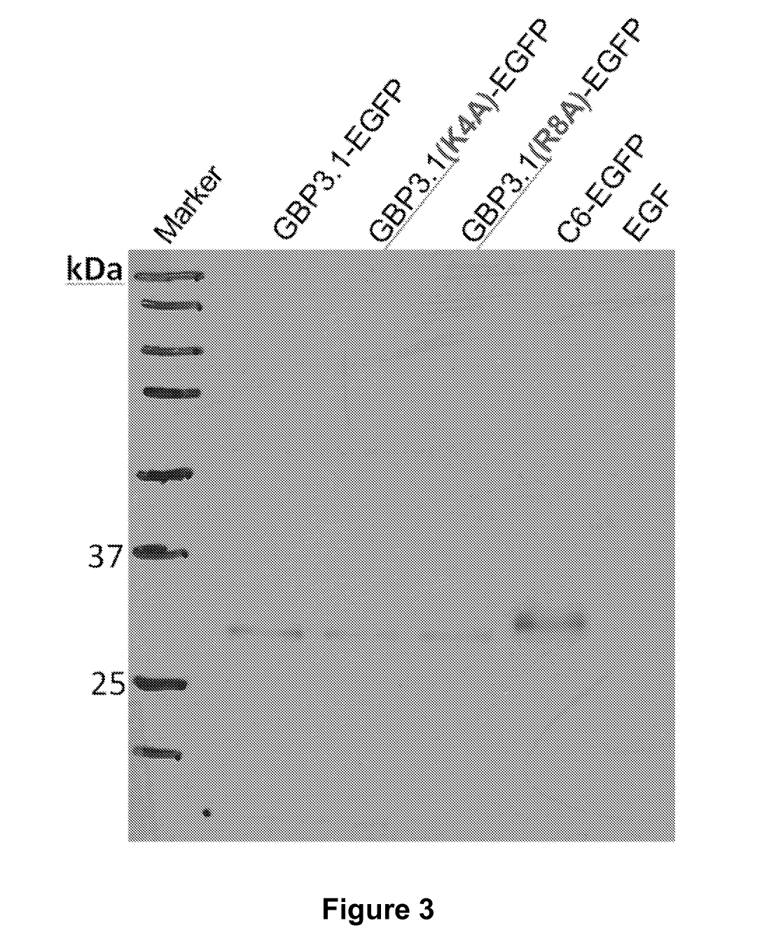

Purification and activation of CGAL1 and CGAL3. Previously selected high expressing clones of CGAL1 and CGAL3 as representative toxins were used for large scale production and purification of the protein. These two constructs have N-terminal His tags to improve the purification efficiency and quality. However purification of His-tagged proteins with a Ni-NTA affinity column was inadequate. Extracted proteins were treated with trypsin under controlled conditions in an attempt to remove contaminating E. coli proteins to improve the ratio of recombinant toxin to E. coli proteins for improved Ni-NTA affinity binding efficiency, but this approach failed to improve purification efficiency. Without wishing to be bound by any particular theory, it is believed that the recombinant toxins may not be present in monomeric form and hence access of the His-tag to the Ni-affinity matrix may be blocked. Hence the original Cyt2Aa purification protocol was used to get sufficient protein to conduct bioassays (FIG. 3). Although the purified proteins clearly contained contaminating proteins (FIG. 3 activation and functional characterization studies of these chimeric protein preparations were carried out using the appropriate control treatments.

For activation of CGAL1 and CGAL3, the amount of trypsin and the incubation time were optimized due to the presence of contaminating proteins: 5% trypsin was used with a 45 min incubation at 37° C., rather than 1% trypsin with a 90 min incubation that is typically used for activation of purified toxin. Trypsin was then removed from samples using Benzamidine Sepharose 6B affinity matrix to avoid potential trypsin-mediated effects in mosquito and pea aphid bioassays. Similar results were obtained with other chimeric toxins described herein (except for those chimeric toxins that were inherently proteolytically unstable.

Because these constructs have a His tag (6 aa) and a five amino acid enterokinase cleavage site along with the twelve amino acid GBP3.1 sequence (SEQ ID NO:11), the theoretical molecular mass of fully processed CGAL1 and CGAL3 is around 25 kDa (Cyt2Aa is 22 kDa). Active CGAL1 and CGAL3 were around 25 kDa with no degraded fragments indicating proteolytic stability of both of these mutant toxins (FIG. 4). The theoretical molecular mass of the pro-mutant toxins is ˜31 kDa. However these protoxins run at ˜27 kDa indicating proteolytic processing before in vitro activation.

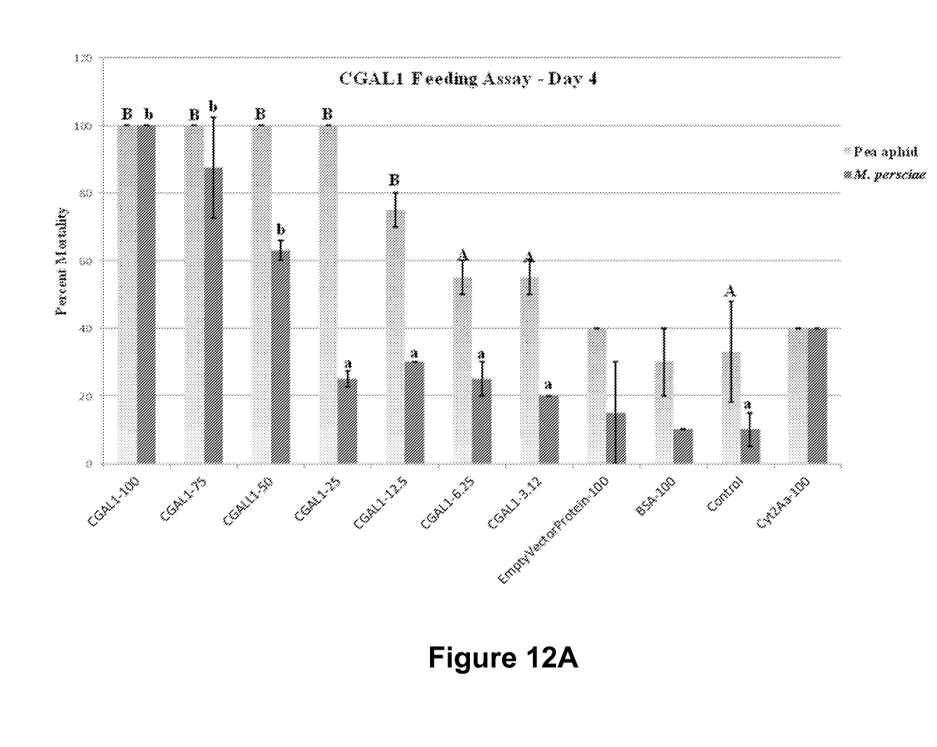

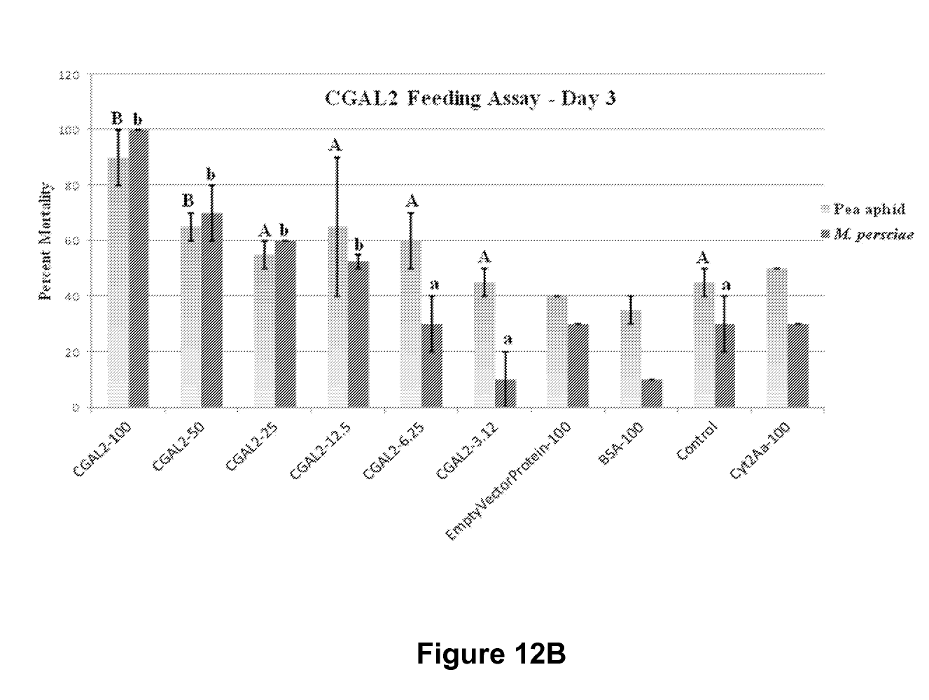

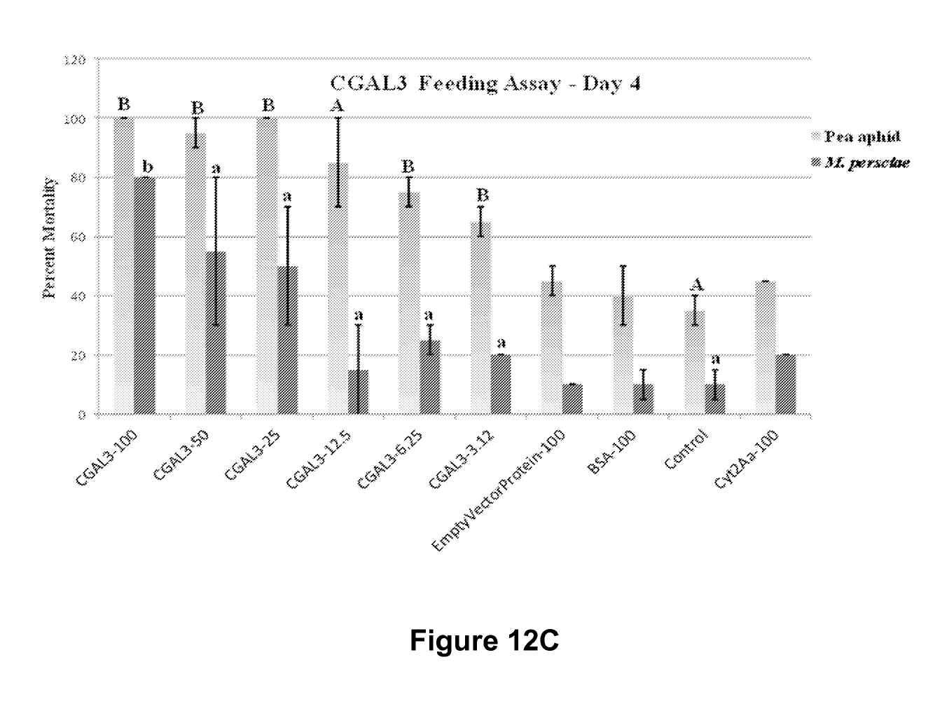

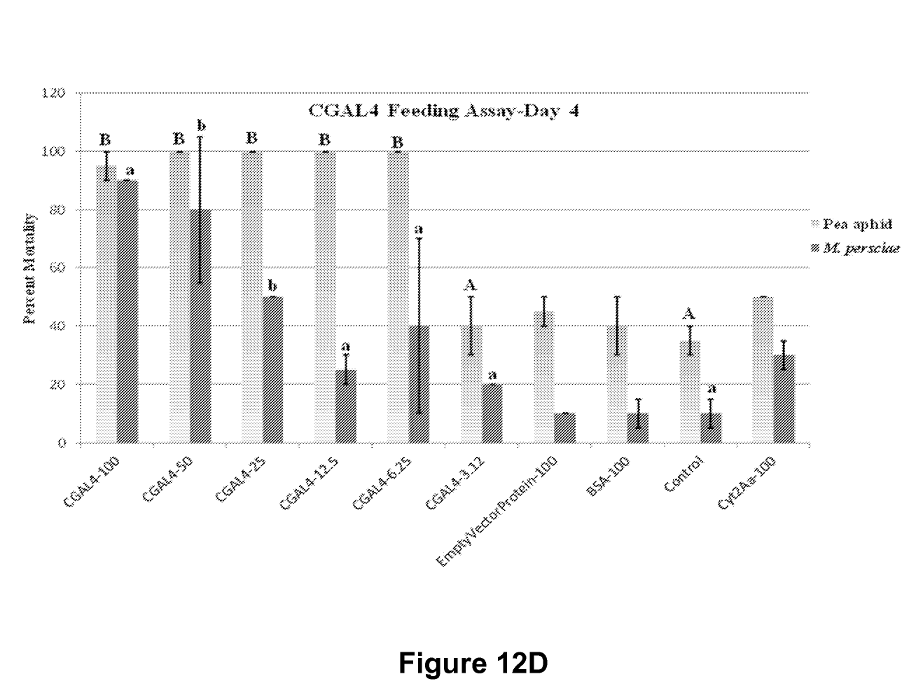

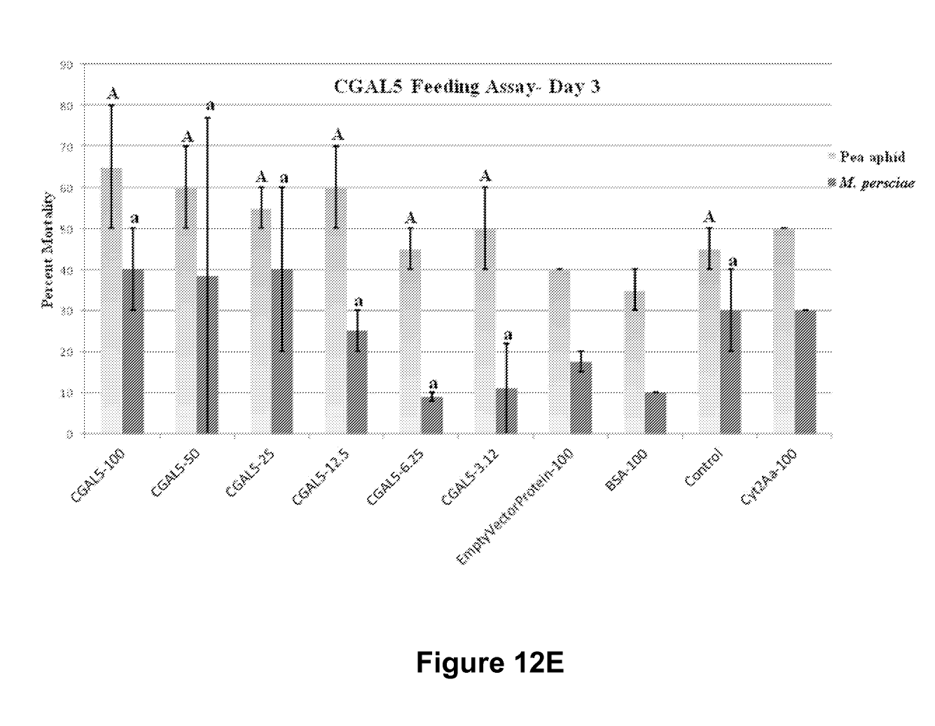

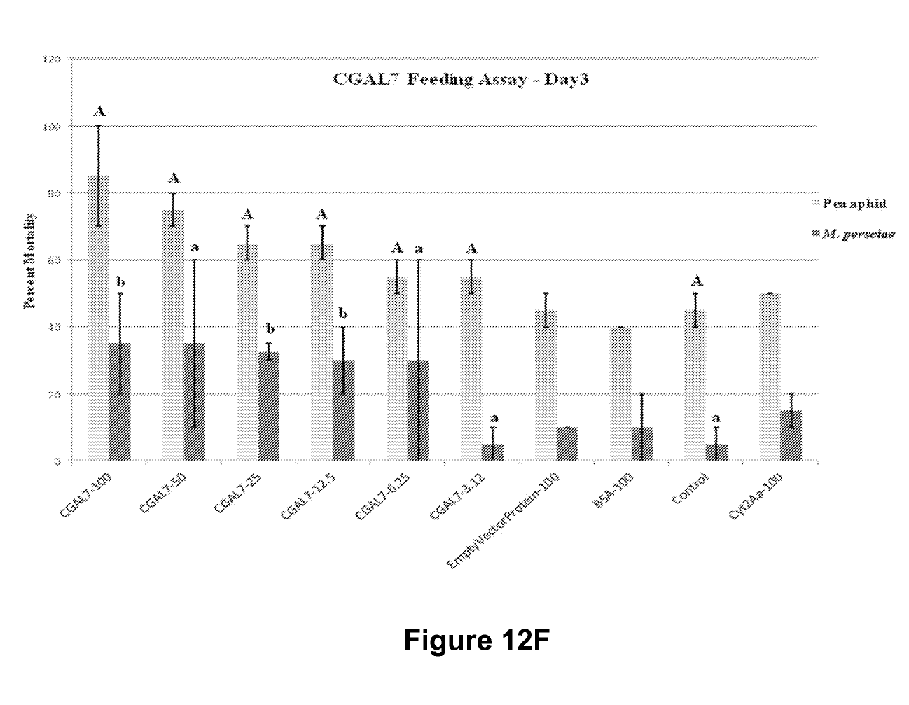

Relative binding of wild type and mutant Cyt2Aa to pea aphid gut membrane. Changes in the ability of Cyt2Aa to bind to pea aphid gut proteins following introduction of GBP3.1 were examined by pull down assay. Very strong binding was seen for active CGAL1 to the whole aphid BBMV whereas no binding was seen for activated wild type Cyt2Aa under optimized experimental conditions (FIG. 12). Binding of active CGAL3 was barely detectable. There was no band present in the BBMV only control lane. Without wishing to be bound by any particular theory, it is believed that the difference in the abilities of active CGAL1 and active CGAL3 to bind to whole aphid BBMV proteins may result from differences in the accessibility of the GBP3.1 peptide; and that the peptide within loop3 in AL3 may be buried within the core structure of Cyt2Aa. The level of toxicity to insects is correlated with the extent of binding of the chimeric protein to the gut membrane. The addition of the gut binding peptide to CytAa at loops 1, 3 and 4 did not affect mosquitocidal activity as compared to the wild type CytAa protein, but the toxicity to aphids was significantly increased as compared to the wild type CytAa protein. However, incorporation of the gut binding peptide described herein at loops 2, 5 and 7 significantly reduced CytAa toxicity to mosquito larvae.

Mosquito larvicidal and aphicidal activity. The biological activity of partially purified and active mutants as well as wild type Cyt2Aa was ascertained by a mosquito feeding assay. Three day old Aedes aegypti larvae were used for the feeding assay. The feeding assay results showed functional activity of CGAL1, CGAL3 and CGAL4 as well as Cyt2Aa, which indicates that introduction of the GBP3.1 peptide into Cyt2Aa did not significantly affect the core structure or functional domains. Chimeric Bt proteins in which the gut binding peptide substituted for a loop of the Bt protein and which exhibited aphicidal activity included CGSL1 and SGSL4. Raw data from this feeding assay were subjected to statistical analysis using PoloPlus software (Table 1). The LC₅₀ values indicate that chimeric toxin CGAL1 is significantly more toxic to mosquito larvae than wild type Cyt2Aa and chimeric CGAL3 protein (ANOVA p=1.7E-05 and p=7.7E-06 respectively). Although CGAL3 showed functional activity against mosquito larvae, the LC₅₀ was significantly lower than that for wild type Cyt2Aa and CGAL1 proteins (ANOVA p=2.8E-05 and p=7.7E-06, respectively).

The increased toxicity of CGAL1 to mosquito larvae suggests that the peptide GBP3.1 may bind to components that are common to the guts of both aphids and mosquitoes. Without wishing to be bound by any particular theory, it is believed that the difference in toxicity of the two Cyt2Aa mutants relates to the stability of the core structure of CGAL3 and/or to accessibility of the introduced GBP3.1 peptide. Addition of the GBP3.1 sequence to loop 1 is less likely to affect the core structure because this loop is at the N-terminal end of the protein. In contrast, homology modeling suggests that loop 3 is in the middle of core structure of Cyt2Aa, and introduction of GBP3.1 may have destabilized the toxin. Alternatively, the accessibility of GBP3.1 within loop 3 may be restricted for binding of CGAL3 to gut membrane proteins. Results from the in vitro pull down assays and analyses of purified proteins appear to support this second possibility.

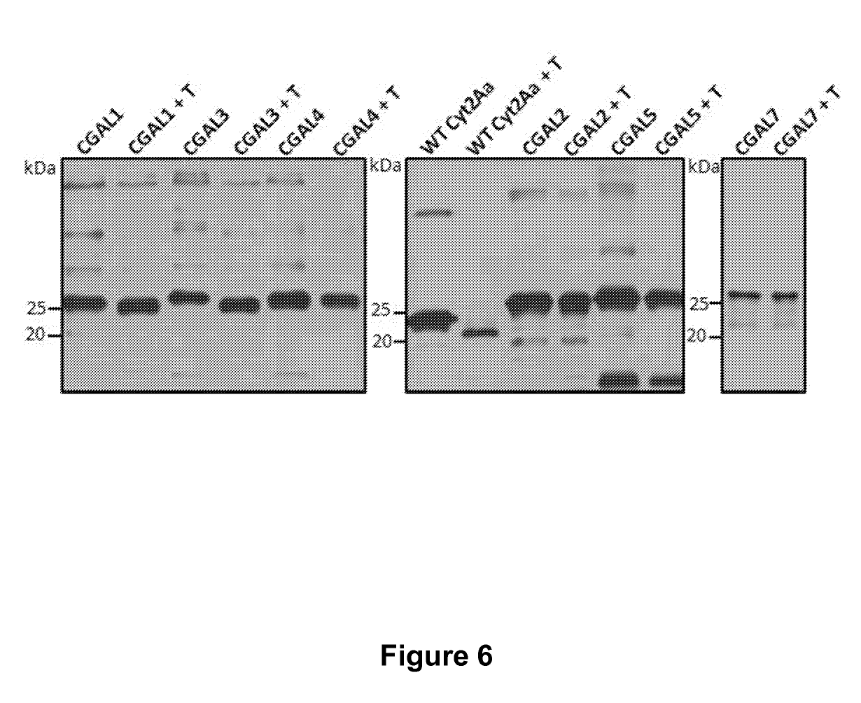

Once the biological activity of the mutant Cyt2Aa had been confirmed in mosquito larvae, feeding assays were conducted to determine the relative toxicity of Cyt2Aa and mutant Cyt2Aa against the pea aphid. A single high concentration of 100 μg/ml was administered in membrane feeding assays using complete artificial diet. Both CGAL1 and CGAL3 in their pro- and active forms were toxic to the pea aphid (FIG. 6). As observed in previous assays, Cyt2Aa was not toxic to pea aphids in the pro- or active toxin form. CGAL1 was significantly more toxic than wild type Cyt2Aa after 60 hr of membrane feeding (ANOVA p=0.000329 and p=0.000432 for pro and active CGAL1, respectively). CGAL3 was also significantly more toxic against the pea aphid toxicity than wild type Cyt2Aa (ANOVA p=0.002215 and p=0.000555 for pro and active CGAL3 respectively). Control treatments of vector proteins and BSA (at the same protein concentrations) were used to assess the possible effects of contaminating E. coli proteins in toxin preparations, and the effect of high protein concentration respectively on pea aphid survival.

TABLE 1
Mosquito larvicidal activity of wild type and certain mutant Cyt2Aa
toxins. PoloPlus statistical software was used to determine the
LC50 values. Values in parentheses show the range of toxin
concentrations used for serial dilutions in feeding assays.

| Toxin | LC50 μg/ml (dose range used) | CL 95% |
|---|---|---|
| Cyt2Aa | 0.488 A (12.5-0.097) | 0.210-0.903 |
| | Slope = 1.118 +/− 0.117; | |
| | Nat Resp = 0.000 +/− 0.000 | |
| Active Cyt2Aa | 0.564 A (12.5-0.097) | 0.186-1.184 |
| | Slope = 1.118 +/− 0.117; | |
| | Nat Resp = 0.000 +/− 0.000 | |
| CGAL1 | 0.217 B (12.5-0.097) | 0.058-0.432 |
| | Slope = 1.118 +/− 0.117; | |
| | Nat Resp = 0.000 +/− 0.000 | |
| Active CGAL1 | 0.258 B (12.5-0.097) | 0.22-0.640 |
| | Slope = 1.118 +/− 0.117; | |
| | Nat Resp = 0.000 +/− 0.000 | |
| CGAL3 | 0.621 C (12.5-0.097) | 0.238-1.301 |
| | Slope = 1.118 +/− 0.117; | |
| | Nat Resp = 0.000 +/− 0.000 | |
| Active CGAL3 | 0.665 C (12.5-0.097) | 0.251-1.373 |
| | Slope = 1.118 +/− 0.117; | |
| | Nat Resp = 0.000 +/− 0.000 | |
| Active CGAL4 | 0.181 D (12.5-0.097) | 0.008-0.822 |
| | Slope = 1.118 +/− 0.117; | |
| | Nat Resp = 0.000 +/− 0.000 | |

The following table shows toxicity of CGAL1, CGAL3 and CGAL4 against both aphids and mosquitoes:

TABLE 2
Toxicity of wild type Cyt2Aa and Cyt2Aa-GBP3.1 chimeric insecticidal toxins
against A. aegypti, A. pisum and M. persicae. Aphid LC50 values
at day 4 (except where indicated) were estimated by probit analysis using
PoloPlus statistical software. The relative LC50 for mosquitoes was calculated
by dividing the mutant toxin LC50 by the wild type Cyt2Aa LC50.

| | Mosquito activity | | | A. pisum toxicity | | M. persicae toxicity | |
|---|---|---|---|---|---|---|---|
| | LC50 | | Relative | LC50 | | LC50 | |
| Toxin | (μg/ml) | CL95% | LC50 | (μg/ml) | CL95% | (μg/ml) | CL95% |
| Cyt2Aa | 0.368 | 0.210-0.903 | 1 | >>150 | ND | >>150 | ND |
| CGAL1 | 0.217 | 0.058-0.432 | 0.58 | 12.809 | 2.51-21.00 | 55.96 | 35.01-65.73 |
| CGAL3 | 0.621 | 0.238-1.301 | 1.68 | 5.807 | 0.65-12.23 | 43.17 | 17.18-83.04 |
| CGAL4 | 0.181 | 0.008-0.822 | 0.49 | 8.93 | 0.83-22.43 | 95.29 | 34.67-152.96 |
| | | | | (Day3) | | | |

Mosquito bioassays were conducted with third instar A. aegypti, nine concentrations of toxin, 30 larvae per dose, with two replicates. The assay was run for 48 hr and LC50 values estimated by probit analysis using PoloPlus (LeOra-Software. 1987). Aphid bioassays were conducted by membrane feeding assay (Chay et al. 1996) with 30 third and fourth instars per treatment. Bioassays were conducted in triplicate and LC50 values determined using the POLO program (Russell et al. 1977).

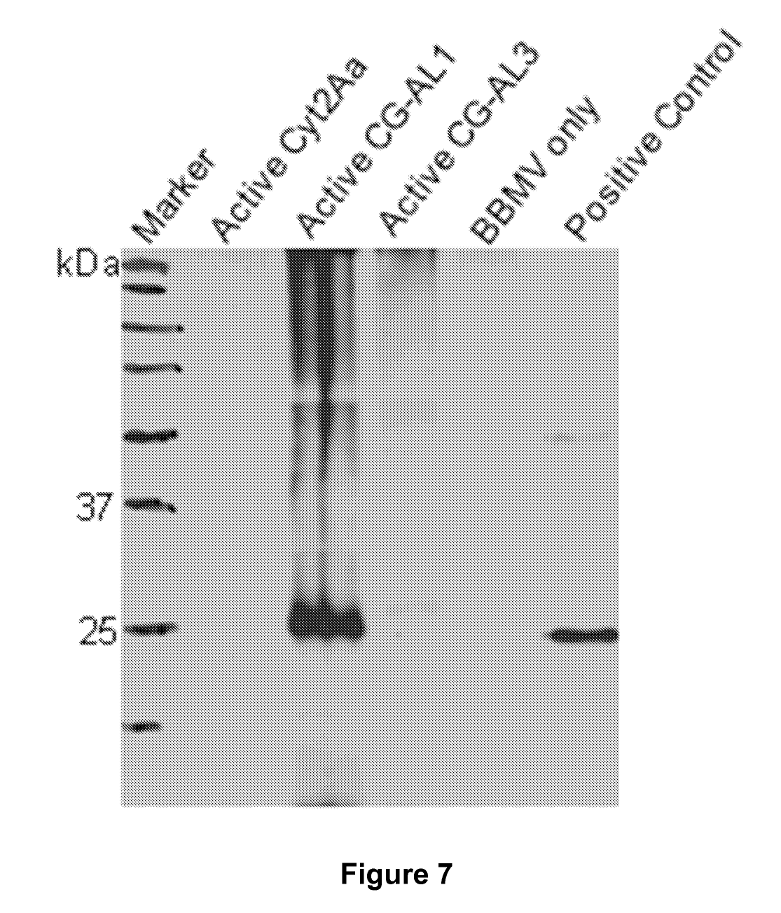

Expression of Cyt2Aa-GBP3.1 addition mutants AL2, AL4, AL5, and AL7. In order to identify mutant Cyt2Aa clones with high expression levels, mutant constructs in pGEM-Teasy (without His tags) were transformed into E. coli BL21 DE3 pLysE. Small scale expression analyses were carried out for comparison with the high expression wild type Cyt2Aa clone. Expression levels of some mutant Cyt2Aa clones were similar to those of wild type Cyt2Aa (FIG. 7). High level expression of these addition Cyt2Aa mutants should facilitate acquisition of sufficient protein for functional characterization. Clone no 2 (C2) of the loop 4 addition mutant (AL4) showed a low mobility band indicating differential proteolytic processing of the recombinant toxin during production.

In summary, partially purified GBP3.1 addition Cyt2Aa mutants (AL1 and AL3) showed correct in vitro proteolytic activation and stability. AL1 showed improved toxicity, while AL3 showed decreased toxicity compared to wild type Cyt2Aa in mosquitocidal assays. AL1 bound strongly and AL3 bound weakly to whole aphid BBMV in pull down assays, while no binding was detected for wild type Cyt2Aa. Preliminary bioassays with pea aphids indicate that AL1 and AL3 are toxic to the pea aphid, while no toxicity was detected for wild type Cyt2Aa against the pea aphid. The increased toxicity of CGAL1 to both mosquito larvae and aphids suggests that GBP3.1 may bind to components that are common to the guts of both aphids and mosquitoes. Retransformation of the remaining four Cyt2Aa addition mutants in E. coli BL21 DE3 pLysE resulted in identification of high expressing clones. In aphid bioassays, CGAL1, CGAL3 and CGAL4 showed similar toxicity, but all were more toxic against the pea aphid than the green peach aphid (Table 2). The relative toxicity compared to wild type Cyt2Aa could not be compared as the Cyt2Aa LC50 could not be estimated under the experimental conditions employed. The highest Cyt2Aa concentrations used (150 μg/ml, and 500 μg/ml), exerted no significant mortality, indicating that the LC50 was well above 150 μg/ml. The CGAL2, CGAL5 and CGAL7 constructs also lacked aphid toxicity at 150 μg toxin/ml (data not shown), likely due to loss of function as indicated by the mosquito bioassays.

In summary, partially purified GBP3.1 addition Cyt2Aa mutants (AL2, AL4 and AL5) showed correct in vitro proteolytic activation and stability. AL2, AL4 and AL5 bound weakly when compared to AL1 to whole aphid BBMV in pull down assays, while no binding was detected for wild type Cyt2Aa.

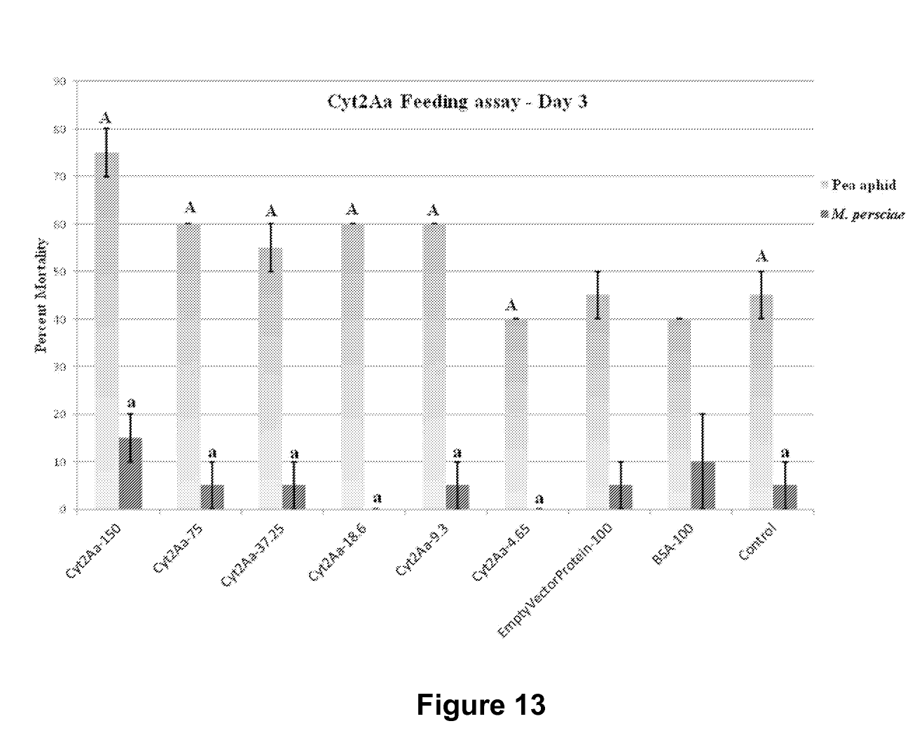

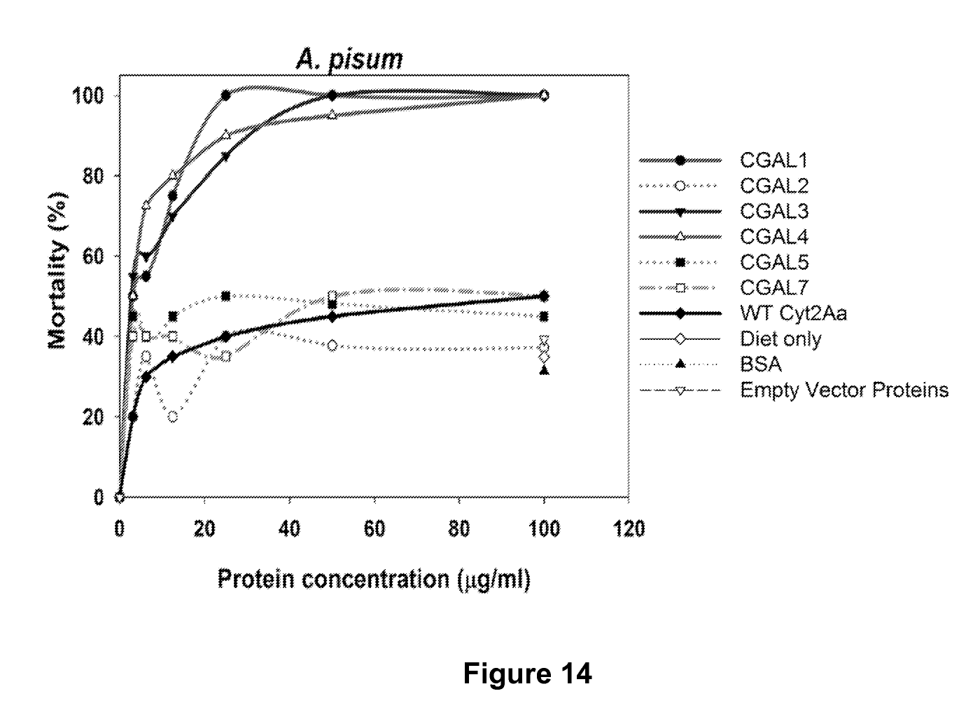

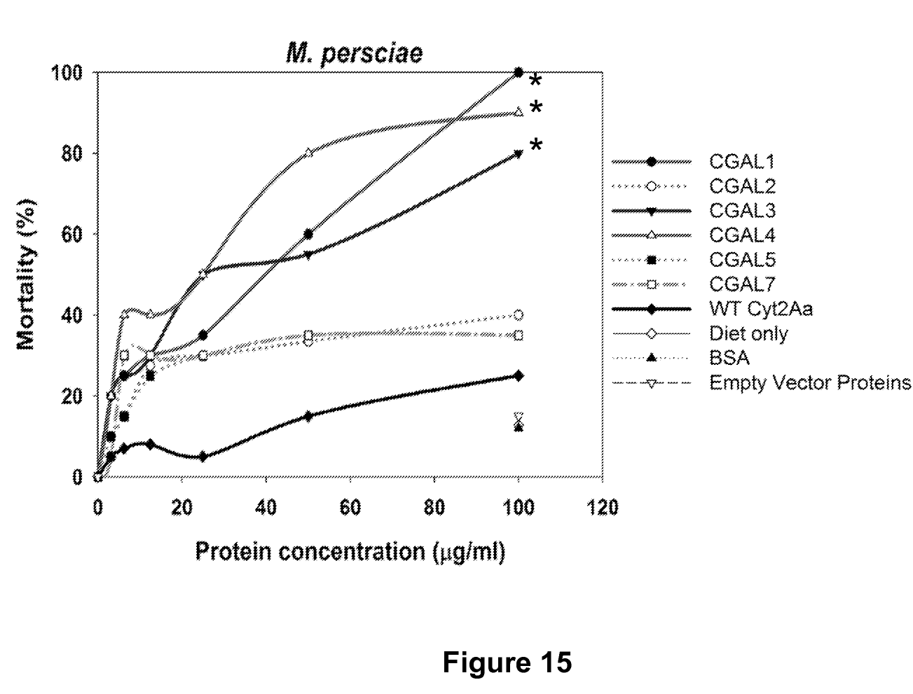

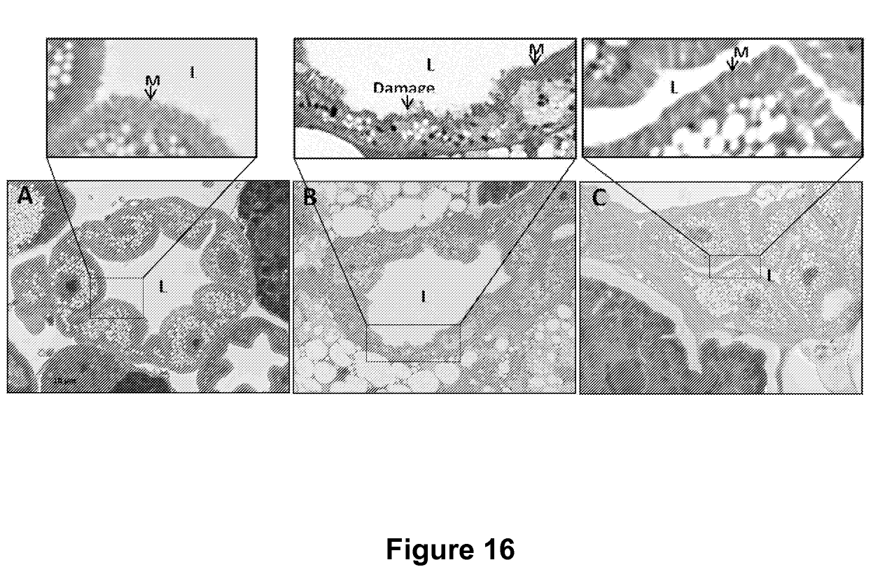

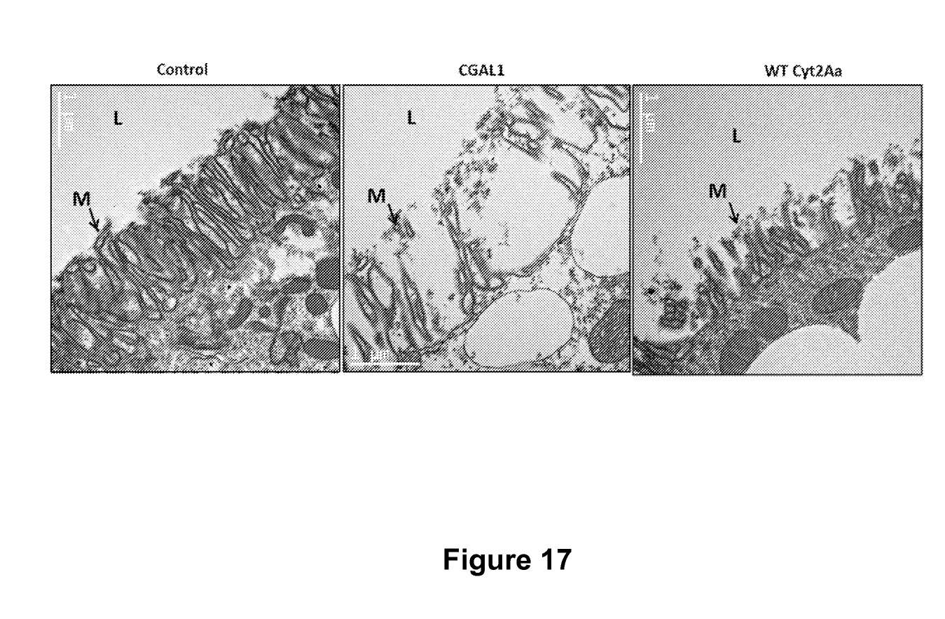

Expression, purification and activation of Cyt2Aa-GBP3.1 substitution mutants CGSL1, CGSL2, CGSL3, CGSL4, CGSL5 and CGSL7. In order to identify mutant Cyt2Aa clones with high expression levels, the original mutant constructs in pGEM-Teasy (without His tags) were transformed into E. coli BL21 DE3 pLysE. Small scale expression analyses were carried out for comparison with the high expression wild type Cyt2Aa clone. Expression levels of some mutant Cyt2Aa clones were similar to those of wild type Cyt2Aa (FIG. 13). These clones were selected for the partial purification of substitution mutants using the original protocol for Cyt2Aa purification (Promdonkoy & Ellar, 2000). E. coli clones expressing either CGAL7 or CGSL7 grow well on antibiotic plates, so it is concluded that the modified toxin has no toxicity to E. coli. However, it is believed that these proteins are unstable, and degrade to a significant degree after expression.

A single colony from a freshly streaked plate was inoculated into 2 ml LB-Carbanicillin and incubated overnight at 37° C. The following day, 500 μl of the overnight culture was inoculated into 500 ml fresh LB medium and incubated at 37° C. until the OD of the culture reached around 0.5 at 600 nm. IPTG was added to the final concentration of 1 mM to induce recombinant protein expression. The culture was incubated at 37° C. for 3-5 hr (250 ml) and 17-20 hr (250 ml) after induction. Cells were pelleted by centrifugation at 3500 rpm for 25 min at 4° C., resuspended in 10 ml of 50 mM Tris-HCl pH 7.5 containing 10 mM KCl and 0.01% Triton X100, 10 mM EDTA and 1 mM PMSF and sonicated on ice 10 times with a 1 min ON/OFF cycle at level 6. The cell lysate was spun at 10,000 rpm for 10 min at 4° C., pelleted inclusion bodies were washed three times with chilled water and the final pellet was solubilized in 1 ml of 50 mM Na2CO3 pH 10.5 buffer at 37° C. for 1 hr. Solubilized toxin was obtained by centrifugation at 10,000 rpm for 10 min at 4° C. The clear supernatant containing recombinant protein was transferred to a fresh tube and checked by SDS-PAGE for purity. Western blot analysis with anti-Cyt2Aa antibodies was also conducted.

Selected high expressing clones of CGSL1, CGSL2, CGSL3 and CGSL4 were used for large scale production and purification of the recombinant proteins. Purification of these mutants was carried out using the standard Cyt2Aa purification protocol (Promdonkoy and Ellar, 2000) to obtain sufficient protein for characterization. As presented in FIG. 2, the mutant toxins were expressed after overnight induction (17-20 hr), except for CGSL4. CGSL4 was expressed in relatively high amounts even after 3-5 hr induction. Partially purified toxins were analyzed by western blot using polyclonal anti-Cyt2Aa antibodies. These partially purified toxins are used for analysis of proteolytic stability, pea aphid gut binding and toxicity assays.

Q-Sepharose FF purification of CGSL1 and other Cyt2A chimeric proteins. Partially purified CGSL1 dialyzed against 50 mM Tri-Cl, pH 8.5 at 4° C. For small scale manual batch purification, dialyzed protein sample was incubated with 1 ml of pre-equilibrated Q-Sepharose FF resin at 4° C. overnight. Toxin bound Q-Sepharose FF resin packed in 7 ml disposable column and washed with 10 column volumes of 50 mM Tri-Cl, pH 8.5 buffer. Column bound toxin was eluted with a manual step gradient ranging from 25 mM to 1 M NaCL in 50 mM Tri-Cl, pH 8.5. One ml elution fractions were collected. All procedures were carried out at 4° C. Eluted fractions were analyzed by western blot for detection of toxin using anti-Cyt2Aa antibodies.

Q-Sepharose-bound CGSL1 protein was eluted between 300-400 mM NaCl concentrations in 50 mM Tris-HCl, pH 8.5. There was no detection of CGSL1 in the flow through or wash fractions, indicating strong binding of the toxin under these conditions.

To express and purify CGSL3 and CGSL7 and other chimeric proteins described herein, a single colony of the relevant transformant from a freshly streaked plate was inoculated and incubated, and chimeric protein expression was induced as described herein above. Soluble toxin protein was prepared as described herein as well.

Purification of all CGSLn proteins provided partially purified proteins along with some contaminating. Partially purified monomeric CGSLn was proteolytically processed during production leading to an intermediate sized toxin of ˜27 kDa as opposed to 29 kDa for the intact mutant toxins. Western blot detection of CGSLn using Cyt2Aa polyclonal antiserum confirmed the presence of protein at ˜27 kDa. As described in a previous report, CGSL3 appears to be highly unstable and degraded to smaller fragments. Two independent partially purified CGSL3 samples appeared to be unstable.

For activation of CGSL7, trypsin was used at a final concentration of 1% of the toxin concentration. Partially purified CGSL7 was dialyzed against 50 mM Tris-Cl pH 8.0 at 4° C. with three buffer changes. Dialyzed CGSL7 was mixed with 1% trypsin in the same buffer and incubated at 37° C. for 45 min. CGSL7 protein without trypsin was included as negative control. SDS sample buffer was added to the reactions and boiled for 5 min and analyzed on 12% SDS-PAGE with western blotting using anti-Cyt2Aa antibodies and CBB staining.

In vitro proteolytic activation of all CGSLn proteins with bovine trypsin did not alter the mobility of the toxin compared to the control (CGSLn without trypsin). In the case of CGSL3, the toxin degraded to fragments of ˜17 and ˜14 kDa even in the absence of trypsin.

Pea aphid and M. persicae feeding assay. Seven protein concentrations (100, 50, 25, 12.5, 6.25, 3.12 and 1.56 μg/ml) of CGSL7 (or other chimeric protein as described herein) were used to test for toxicity against the pea aphid, A. pisum and the green peach aphid, M. persicae. Controls of vector protein only (100 μg/ml) and BSA (100 μg/ml) were included in bioassays to observe the effect of contaminating proteins as well as high protein concentration on the aphids, respectively. Wild type Cyt2Aa (100 μg/ml) and a diet only control were included in the bioassay for comparative purposes. Partially purified CGSL7 was dialyzed against 50 mM Tris-Cl pH 8.0 before use in the feeding assay. Complete artificial liquid diet (Febvay et al., 1987) and second instar pea aphids were used in a growth chamber at 24° C. with an 18:6 light:dark photoperiod. Assays were set up in duplicate with ten aphids per replicate. Mortality was recorded every 24 hr and diet was replaced every third day. The feeding assay was continued for a period of 7 days.

Purification and activation of CGSL7. A previously selected clone with a high level of CGSL7 expression was used for large scale production and purification of the protein. Purification of CGSL7 was carried out using the standard Cyt2Aa purification protocol (Promdonkoy and Ellar, 2000). Purification of CGSL7 provided a better yield relative to CGAL7 with contaminating proteins present at similar levels. Western blot analysis of partially purified CGSL7 using anti-Cyt2Aa antiserum detected monomeric as well as dimeric forms of CGSL7 (not shown). It appears that partially purified monomeric CGSL7 has been proteolytically processed during the production or purification process leading to formation of intermediate size of ˜27 kDa as opposed to 29 kDa of intact CGSL7.

In vitro proteolytic activation of CGSL7 using bovine trypsin produced the active form of CGSL7 with the correct size. However, trypsin treatment also produced some degradation fragments indicative of proteolytic instability of CGSL7 with trypsin. Western blot of activated CGSL7 showed at least three degradation fragments resulting from trypsin cleavage. The control CGSL7 reaction without trypsin also showed three low intensity bands of different sizes. These degraded fragments were not detected in purified CGSL7 which indicates that degradation of CGSL7 occurred during incubation at 37° C. for 45 min due to the activity of trypsin and/or co-purified bacterial protease.

Without wishing to be bound by theory, it is believed that the CGSL7 and CGSL3 proteins are proteolytically unstable. CGS7 did not appear to be toxic to the pea aphid or the green peach aphid in membrane feeding studies. The lack of significant toxicity is believed due to the instability of the protein.

There was no significant difference in mortality between aphids fed on CGSL7 and the negative control treatment except for the 100 μg/ml concentration of CGSL7. While 100 μg/ml CGSL7 resulted in a significant increase in mortality of both the pea aphid and M. persicae when compared to the control treatment, toxicity was not concentration dependent (FIG. 12F). Proteolytic instability may contribute to the low aphid toxicity of CGSL7.

Partially purified CGSL3 was obtained from large scale purification. Contaminating proteins were at similar levels to previous Cyt2Aa mutant purification experiments. Western blot analysis of partially purified CGSL3 showed dimeric, intact monomeric and intermediate active forms of CGSL3, as well as three high intensity bands at around 17, 15 and 12 kDa, which appear to be degraded fragments. The relative intensity of the degraded fragments was higher than that of intact or intermediate active CGSL3 indicating that most of the toxin was degraded.

Pea aphid feeding assay with CGSn (including CGSL1, CGSL2, CGSL4 and CGSL5). Six protein concentrations (100, 50, 25, 12.5, 6.25, and 3.12 μg/ml) of partially purified mutant toxins and wild type Cyt2Aa were used to test for toxicity against the pea aphid, A. pisum. Controls of vector protein only (100 μg/ml) and BSA (100 μg/ml) were included in bioassays to observe the effect of contaminating proteins as well as high protein concentration on the aphids, respectively. A diet only control was included in the bioassay for comparative purposes (Febvay et al., 1987). The protein preparation was neutralized to pH 7.5 with 1 N HCl before use in the feeding assay. The assay was set up in a growth chamber at 24° C. with an 18:6 light:dark photoperiod. Mortality was recorded every 24 hr and diet was replaced every third day. The feeding assay was continued for a period of 7 days. LC50 was estimated by probit analysis using PoloPLus.

In vivo binding of CGALn and CGSLn (including CGAL1 and CGAL3) to pea aphid gut membrane. A single protein concentration of 25 μg/ml of CGAL1, CGAL3, wild type Cyt2Aa was fed to pea aphids in complete liquid diet in a membrane feeding assay for 24 hr. Control aphids were fed on diet only. Forty pea aphids per treatment were included in the feeding assays. Pea aphid guts were dissected from 30 insects from each set and homogenized in 100 μl of 1×PBS in an eppendorf tube. The membrane fraction was isolated by centrifugation at 12,000 rpm for 10 min at 4° C. The pellet was washed thrice with 1×PBS, resuspended in 10 μl of 1×SDS-Sample buffer, boiled for 5 min and loaded on 12% SDS-PAGE gels. Toxin associated with the pea aphid gut membrane was detected using Cyt2Aa polyclonal antiserum.

Pea aphid gut BBMV binding assay. Pea aphid gut BBMV (10 μg) were incubated with 50 nM activated Cyt2Aa, or activated CGAL1, CGAL2, CGAL3, CGAL4, CGAL5 and CGAL7 in 100 μl binding buffer (1×PBS pH 7.4, 0.1% BSA, 0.1% Tween20) for 1 hr at room temperature, centrifuged at 14,000 rpm for 15 min at 4° C. The pelleted BBMV were washed three times with 500 μl of binding buffer. The final BBMV pellet was resuspended in 10 μl 1×SDS sample buffer, boiled for 5 min, separated by 12% SDS-PAGE and bound proteins detected by western blot using anti-Cyt2Aa antibodies. This experiment was repeated twice.

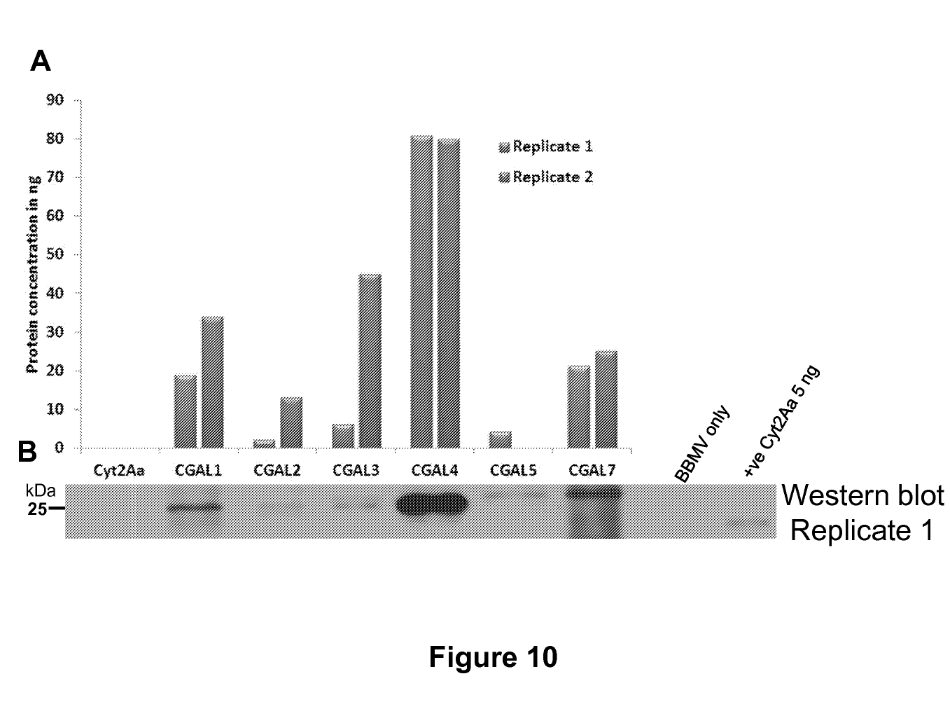

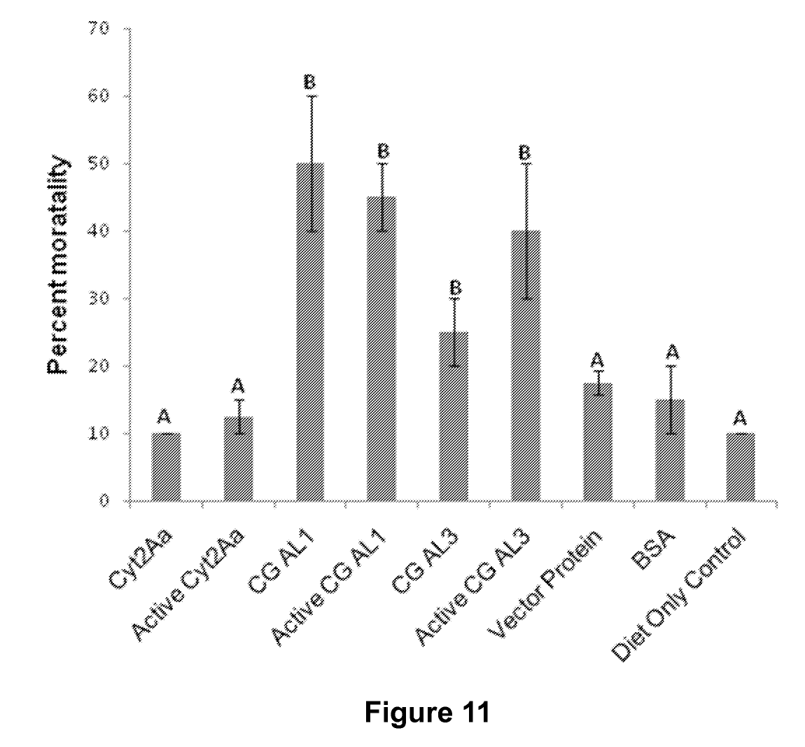

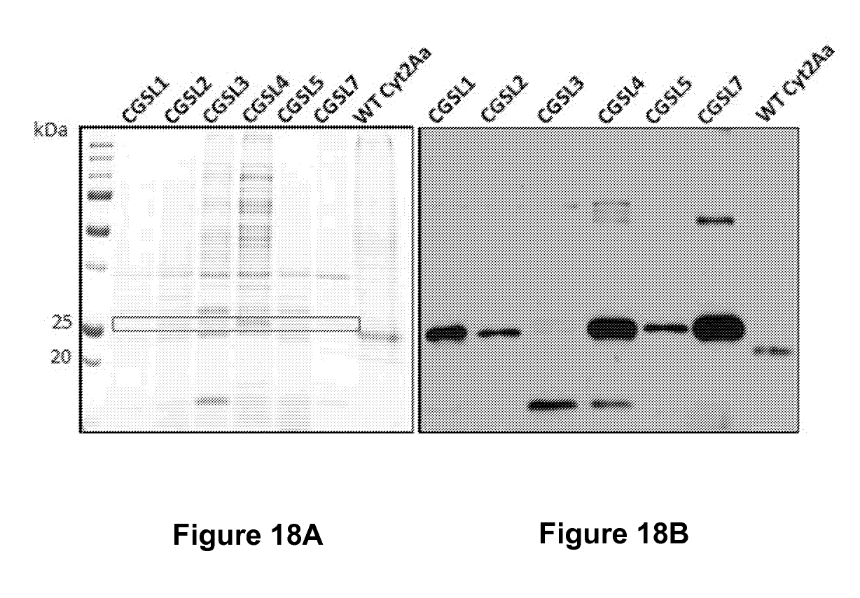

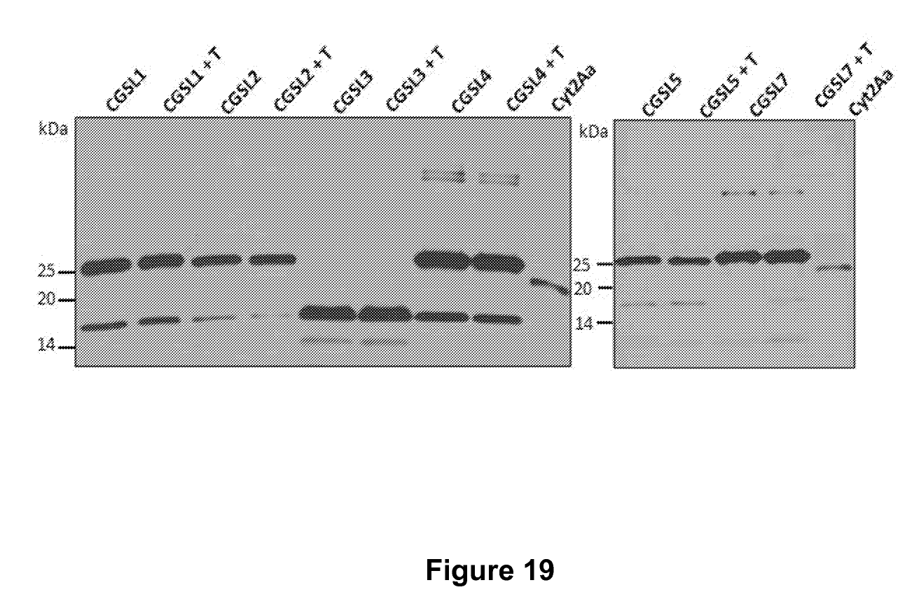

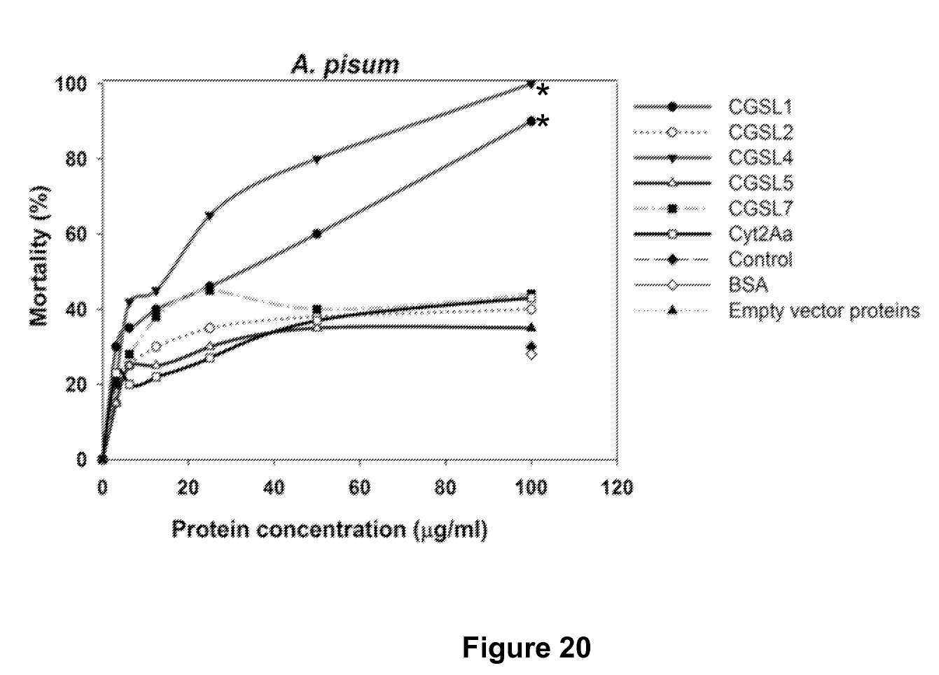

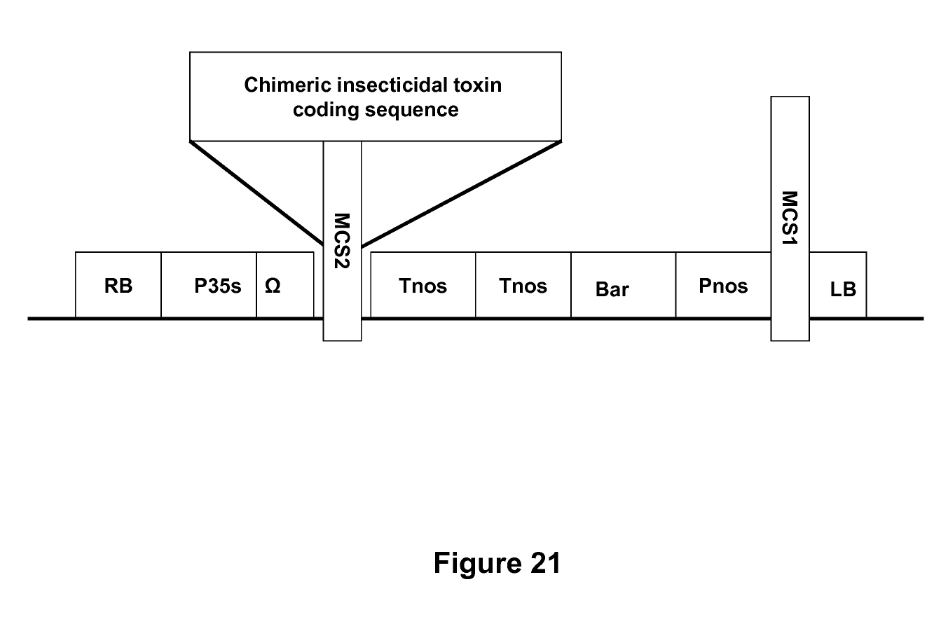

Toxicity of CGSL1, CGSL4 and CGSL5 against the pea aphid. Data presented in FIGS. 18-10 include data from day 2 of the feeding assay, whereas day 3 data were used for estimation of LC50 values. Control mortality on day 3 was 40% however. Both CGSL1 and CGSL4 showed a concentration dependent effect on the pea aphid (FIG. 20). The LC50 values for CGSL1 and CGSL4 against the pea aphid were 32.95 and 5.807 μg/ml respectively (Table 3). Studies indicated improved toxicity of CGSL1 and CGSL4 but not CGSL2, CGSL5 and CGSL7. CGSL3 was highly unstable, and hence it was not included in aphid toxicity assays. The in vivo pea aphid gut binding of five CGSLn mutants, excluding CGSL3, has been tested.

The relative toxicity could not be compared with wild type Cyt2Aa as the LC50 for Cyt2Aa could not be estimated under the experimental conditions employed.

TABLE 3
Comparative toxicity analysis of wild type Cyt2Aa and CGSL1, CGSL4,
CGSL5 substitution mutants against pea aphid. LC50 were estimated
from day 3 data by probit analysis using PoloPlus statistical software.
Mortality in control treatments by day 3 was 40%.

Pea aphid toxicity

| Toxin | LC50 (μg/ml) | 95% CL |
|---|---|---|
| Cyt2Aa | ND | ND |
| CGSL1 | 32.95 | 6.40-93.40 |
| CGSL4 | 14.88 | 4.3-25.26 |
| CGSL5 | ND | ND |

ND, could not be determined under experimental conditions used

Modification of Cyt2Aa amino acid residues in loops 1 and 4 does not affect the pore forming activity and hence the toxicity of Cyt2Aa (Promdonkoy and Ellar, 2000; Promdonkoy and Ellar, 2005). Without wishing to be bound by theory, it is believed that substitution or addition of GBP3.1 to loops 1 and 4 of Cyt2Aa does not affect the toxicity. The LC50 values of CGSL4 and CGAL4 are similar (14.88 and 10.03 μg/ml respectively) whereas the LC50 values for CGSL1 and CGAL1 differed (32.95 and 14 μg/ml respectively). CGSL5 showed some marginal improvement in toxicity when compared to wild type Cyt2Aa (FIG. 20), but LC50 values could not be calculated for this feeding assay

In vivo binding of CGAL1 and CGAL3 to pea aphid gut membrane. Proteolytic processing of toxins in the gut and the relative binding of the mutant toxins to the aphid gut membrane was assessed.

The membrane fraction derived from 30 aphid guts was loaded in each lane. The results show binding of both CGAL1 and CGAL4 to the pea aphid gut membrane. The observed size of the bound toxin was ˜22 kDa, indicating that these toxins were proteolytically processed by aphid gut proteases. A faint band of ˜21 kDa was also observed for wild type Cyt2Aa-fed pea aphid gut samples (FIG. 6). No toxin band was detected in the control aphids fed diet alone. A non-specific band of 31 kDa was observed in all four treatment groups. A small aliquot of aphid diet plus toxin was removed and incubated in an eppendorf tube for the same time and temperature to check toxin stability toxin. The results indicated that the toxins were stable in aphid diet for 24 hr. This result confirmed that the toxins were processed in the aphid gut to their active, stable form.

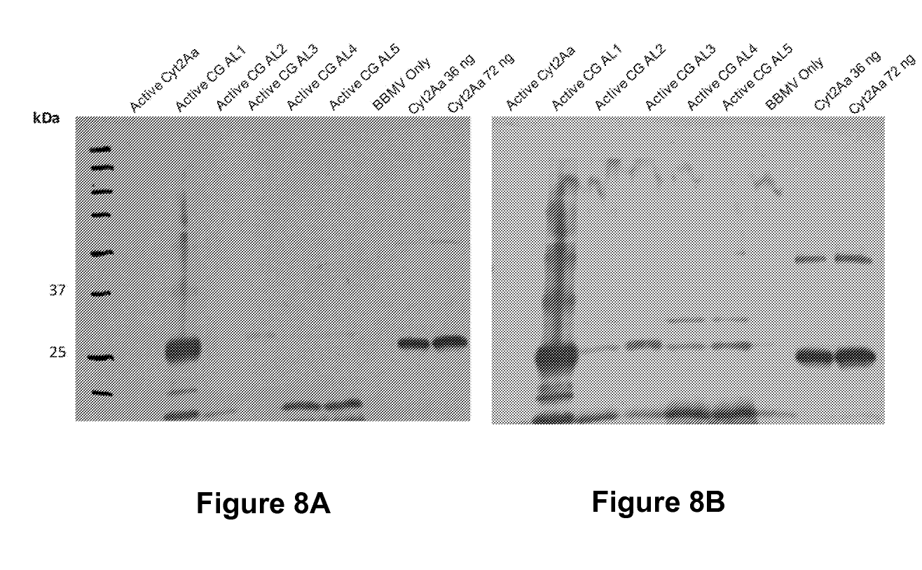

Relative binding of wild type Cyt2Aa and CGALn to pea aphid gut BBMV The ability of Cyt2Aa to bind to pea aphid gut proteins following introduction of GBP3.1 was examined by pull down assay. FIGS. 8-10 shows binding of CGALn mutants from two replicates. More CGAL4 bound to aphid gut BBMV than other CGALn mutants. Cyt2Aa binding was not detected. CGAL1, CGAL4 and CGAL7 showed relatively high binding in both replicates whereas CGAL3 showed high binding in a single replicate. CGAL5 showed very limited binding to the pea aphid gut BBMV. Note that CGAL7 appears to be a somewhat unstable protein, as analyzed by Western blotting.

Mosquito feeding assays. Third instar Aedes aegypti larvae were used in the feeding assay to estimate the relative toxicity of purified CGSL1, CGSL2, CGSL4, CGSL5, CGSL7 and wild type Cyt2Aa. The assay was set up in a 24-well cell culture plate with 1 ml of protein solution in distilled water in each well. Toxin dilutions ranged from 100 μg/ml to 0.195 μg/ml in serial two-fold dilutions. Six different groups (control, CGSL1, CGSL2, CGSL4, CGSL5 and CGSL7 and vector protein control) were set up in duplicate with 10-15 larvae per well. The protein preparation was neutralized to pH 7.5 with 1 N HCl before being used in the feeding assay. Plates were incubated in an incubator at 28° C. with 75% humidity and a 18:6 light:dark photoperiod. Mortality of larvae was recorded every 24 hr and the assay was run for 48 hr. LC50 was estimated by probit analysis using PoloPlus.

Analysis of the impact of CGSL4 on the aphid gut Second instar pea aphids were fed on a single concentration (100 μg/ml) of CGSL4 or wild type Cyt2Aa in complete artificial diet by membrane feeding. Control aphids were fed on diet alone. The assay was set up in triplicate with ten aphids per replicate in a growth chamber at 24° C. with an 18:6 light:dark photoperiod. After a period of approximately 72 hr, aphids from all groups were collected. The rear abdomen was cut and aphids were fixed in a fixative solution containing embedded resin and subjected to microscopic analysis.

Mosquitocidal activity of CGSL1, CGSL2, CGSL4, CGSL5 and CGSL7 To confirm the functional activity of the substitution mutants, CGSL1, CGSL2, CGSL4, CGSL5 and CGSL7, A. aegypti feeding assays were carried out with nine different concentrations for LC50 estimation. Results presented in Table 4 indicate that CGSL1 and CGSL4 maintained toxicity, similar to wild type Cyt2Aa. However, the remaining three mutants, CGSL2, CGSL5 and CGSL7 showed a decrease in toxicity against A. aegypti and the LC50 values could not be estimated with the protein concentration range used (100 to 0.195 μg/ml).

The decrease in functional activity of the three substitution mutants, CGSL2, CGSL5 and CGSL7 was similar to that of the CGAL2, CGAL5 and CGAL7. Without wishing to be bound by any particular theory, it is believed that any changes to loops 2, 5 and 7 affects the control toxicity of Cyt2Aa. In fact, structure-function studies on Cyt2Aa implicated amino acids in loops 2, 5, and 7 in pore formation. Addition or substitution of GBP3.1 to these loops is believed to have altered the pore forming toxin structure to the extent that the toxin loses its pore forming ability. Structure-function studies of Cyt2Aa indicated that (i) amino acids in loop 2 and the loop 2 flanking helices (aA and aB) are important for pore formation (Promdonkoy and Ellar, 2005); (ii) amino acids in loop 5 and the loop 5 flanking β5 and β6 are involved in pore formation and are inserted into the membrane (Promdonkoy and Ellar, 2000; Promdonkoy and Ellar, 2005; Promdonkoy et al., 2008); (iii) two amino acids from β7, which is at the N-terminal end of loop 7 are inserted into the membrane during pore formation (Promdonkoy and Ellar, 2005). The loss of CGAL7 toxicity may result from the fact that GBP3.1 is located next to β7 which may affect the pore forming ability of the toxin.

TABLE 4
Comparative toxicity analysis of wild type Cyt2Aa and CGSLn mutants
against A. aegypti and pea aphid LC50 values were estimated
for mortality at day 3 by probit analysis using PoloPlus statistical
software. The relative LC50 for mosquitoes was calculated by dividing
the mutant toxin LC50 by the wild type Cyt2Aa LC50.

| | Mosquitocidal activity | | | Pea aphid toxicity | |
|---|---|---|---|---|---|
| | LC50 | | Relative | LC50 | |
| Toxin | (μg/ml) | CL95% | LC50 | (μg/ml) | CL95% |
| Cyt2Aa | 0.295 | 0.103-0.890 | 1 | ND | ND |
| CGSL1 | 0.358 | 0.192-0.793 | 1.21 | 32.95 | 6.40-93.40 |
| CGSL2 | ND | ND | ND | ND | ND |
| CGSL4 | 0.402 | 0.116-0.915 | 1.36 | 14.88 | 4.3-25.26 |
| CGSL5 | ND | ND | ND | ND | ND |
| CGSL7 | ND | ND | ND | ND | ND |

ND, not determined.

Toxicity of CGSL2 against the pea aphid Data presented in FIG. 20 are from day 2 of the pea aphid feeding assay. CGSL2 showed no improvement in the toxicity to pea aphids compared to wild type Cyt2Aa. The LC50 values for CGSL2 against the pea aphid could not be determined under the experimental conditions employed, likely due to the loss of mosquitocidal activity.

In vivo binding of CGSLn to pea aphid gut. Proteolytic processing of toxins in the gut and to assess the relative binding of the mutant toxins to the aphid gut membrane. The membrane fraction derived from 30 aphid guts was loaded in each lane. The results show binding of CGSL1 and CGSL4 to the pea aphid gut membrane. The observed size of the bound toxin was ˜22 kDa, indicating that these toxins were proteolytically processed by aphid gut proteases. In the case of CGSL2, CGSL5 and CGSL7, no toxin specific band was detected indicating no binding of these mutants to pea aphid gut membrane. However, a faint band of ˜21 kDa which was observed for wild type Cyt2Aa-fed pea aphid gut samples in the previous experiments, was not clearly visible in this experiment. This could be due to variability in pea aphid feeding. No toxin band was detected in the control aphids fed diet alone. A nonspecific band of 31 kDa was observed in all treatment groups. A small aliquot of aphid diet plus toxin was removed and incubated in an eppendorf tube for the same time and temperature to check toxin stability toxin. The results indicated that the toxins were stable in aphid diet for 24 hr. This result confirms that the chimeric toxins were processed in the aphid gut to their active, stable form.

Damage to Insect Gut. Transmission electron microscopic analysis of CGAL1-fed aphids revealed that there was clear damage to the microvillar structure of the gut, with almost complete loss of microvilli from the gut membrane. Wild-type Cyt2a also appeared to cause some damage to the gut microvilli as compared to the control aphid guts (fed with normal diet only).

Plant Transformation. Additional embodiments of the invention relate to transformed seeds and transgenic progeny plants of the parent transgenic plant, all expressing the gut-binding-peptide, peptide multimer or a fusion protein comprising same, advantageously expressed in the phloem of the plants, and the use of said plants, seeds, and plant parts in agro-industry and/or horticulture and/or in the production of food, feed, industrial products, oil, nutrients, and other valuable products. These other embodiments of the invention relate to transformed seed of such a plant, methods for breeding other plants using said plant, use of said plant in breeding or agriculture, and use of said plant to produce chemicals, food or feed products, as well as to reduce transmission of targeted virus diseases spread by sap-sucking insects and to reduce plant damage and economic losses due to those targeted virus diseases. The expression of the gut-binding peptide, peptide multimer or a fusion protein comprising same reduces the spread of viruses carried by sap-sucking insects, luteoviruses, geminiviruses and especially enamoviruses such as Pea enation mosaic virus carried by aphids, and as a result, the infection of plants by such viruses is reduced and crops expressing the peptide, peptide multimer or fusion protein are afforded some level of protection against damage due to such viral infection. The use of transgenic plants expressing the peptide, peptide multimer or fusion protein among, near or surrounding a nontransgenic plant of interest also affords some protection to the nontransgenic plant of interest.

For recombinant production of the peptide in a host organism, the gut binding, plant virus inhibiting peptide coding sequence is inserted into an expression cassette designed for the chosen host and introduced into the host where it is recombinantly produced. The choice of specific regulatory sequences such as promoter, signal sequence, 5′ and 3′ untranslated sequences, and enhancer, is within the level of skill of the one ordinarily skilled in the art. The resultant molecule, containing the individual elements linked in proper orientation and reading frame, may be inserted into a vector capable of being transformed into the host cell. Suitable expression vectors and methods for recombinant production of proteins are well known for host organisms such as E. coli (see, e.g., Studier and Moffatt. 1986. J. Mol. Biol. 189: 113; Brosius. 1989. DNA 8: 759), yeast (see, e.g., Schneider and Guarente. 1991. Meth. Enzymol. 194: 373) and insect cells (see, e.g., Luckow and Summers. 1988. Bio/Technol. 6: 47). Specific examples include plasmids such as pBluescript (Stratagene, La Jolla, Calif.), pFLAG (International Biotechnologies, Inc., New Haven, Conn.), pTrcHis (Invitrogen, Carlsbad, Calif.).

Plants expressing a chimeric insecticidal toxin described herein can be obtained by stably transforming a peptide coding sequence of the present invention into a plant cell such that it is expressed in the above-ground plant tissues, and preferably in phloem, and is stably maintained in the plant.

As specifically exemplified herein, the plant used for transgenic expression of GBP3.1-linked Cyt2Aa is pea (Pisum sativum ssp.). Other dicotyledonous species, especially legumes, and importantly soybean, vegetables, tobacco, grains, fodder, ornamental plants such as members of the rose family, among other plants, can be similarly constructed, using plant transformation and regeneration technology well known to the art. Monocots susceptible to attack by aphids and viruses can also be made.

Agrobacterium tumefaciens-mediated transformation is used to make aphid-resistant plants, for example legumes such as soybean or other bean, vegetables such as tomatoes or potatoes, crops such as oilseed, tobacco and other Nicotiana species, and ornamental plant such as roses. T-DNA binary vectors are used for introducing the plant-expressible sequences encoding a gut binding peptide, peptide multimer or fusion protein of the present invention. Embryonic segments from mature pea seeds are used as initial explants. Alternatively, stem segment and axillary buds may also be used; see Krejci et al., 2007. The transformation of pea (Pisum sativum L.): applicable methods of A. tumefaciens-mediated gene transfer. Acta Physiol. Plant. 29:157-163, which provides methods for preparing and transforming embryonic segments, and for regeneration of transformant plants from the callus tissue. See also Jordan et al. 1993. Evaluation of a cotyledonary node regeneration system for Agrobacterium-mediated transformation of pea (Pisum sativum L.). In Vitro Cell Dev. Biol. 29:77-82.

The strategy for construction of the vectors is summarized in FIG. 20. See also Xiao et al., 1999. A mini binary vector series for plant transformation. Plant Molecular Biology 40: 711-717.

PCR is used to confirm that the transgenic plants contain the proper insertion of the chimeric toxin gene. RT-PCR is used to confirm that the peptide, tandem repeat or fusion protein mRNA is transcribed, and ELISA or western blots establish that the protein is expressed.

The effectiveness of the expressed chimeric insecticidal toxin is shown by bioassay of the transformed plants, as described herein.

Examples of constitutive promoters which function in plant cells include the Cauliflower mosaic virus (CaMV) 19S or 35S promoters, CaMV 35S double or enhanced promoters, the 35S promoter and an enhanced or double 35S promoter such as that described in Kay et al., Science 236: 1299-1302 (1987); nopaline synthase promoter; the rice actin promoter (McElroy et al. 1991. Mol. Gen. Genet. 231: 150), maize ubiquitin promoter (EP 0 342 926; Taylor et al. 1993. Plant Cell Rep. 12: 491), and the Pr-1 promoter from tobacco, Arabidopsis, or maize (see U.S. Pat. No. 5,614,395), the Peanut chlorotic streak caulimovirus (PCISV) promoter (U.S. Pat. No. 5,850,019), the 35S promoter from Cauliflower mosaic virus (CaMV) (Odell et al. 1985. Nature 313:810-812), promoters of Chlorella virus methyltransferase genes (U.S. Pat. No. 5,563,328), the full-length transcript promoter from Figwort mosaic virus (FMV) (U.S. Pat. No. 5,378,619); the promoters from such genes as rice actin (McElroy et al. 1990. Plant Cell 2:163-171), ubiquitin (Christensen et al. 1989. Plant Mol. Biol. 12:619-632) and Christensen et al. 1992. Plant Mol. Biol. 18:675-689), pEMU (Last et al. 1991. Theor. Appl. Genet. 81:581-588), MAS (Velten et al. 1984. EMBO J. 3:2723-2730), maize H3 histone (Lepetit et al. 1992. Mol. Gen. Genet. 231:276-285 and Atanassova et al. 1992. Plant Journal 2:291-300), Brassica napus ALS3 (WO 97/41228); and promoters of various Agrobacterium genes (see, e.g., U.S. Pat. Nos. 4,771,002, 5,102,796, 5,182,200 and 5,428,147). Light-regulated promoters suitable for expression in above-ground tissues include the small subunit of ribulose bisphosphate carboxylase (ssuRUBISCO) promoter and the like. The promoters themselves may be modified to manipulate promoter strength to increase peptide, peptide multimer or fusion protein expression, in accordance with art-recognized procedures.

Guidance for the design of promoters is provided by studies of promoter structure, such as that of Harley and Reynolds. (1987). Nucleic Acids Res. 15:2343-2361. Also, the location of the promoter relative to the transcription start may be optimized. See, e.g., Roberts, et al. (1979) Proc. Natl. Acad. Sci. USA 76:760-4. Many suitable promoters for use in plants are well known in the art.

The promoter may include or be modified to include one or more enhancer elements. Promoters with enhancer elements provide for higher levels of transcription as compared to promoters without them. Suitable enhancer elements for use in plants include the PCISV enhancer element (U.S. Pat. No. 5,850,019), the CaMV 35S enhancer element (U.S. Pat. Nos. 5,106,739 and 5,164,316) and the FMV enhancer element (Maiti et al. (1997). Transgenic Res. 6:143-156). See also WO 96/23898 and Enhancers and Eukaryotic Expression (Cold Spring Harbor Press, Cold Spring Harbor, N.Y., 1983).

A 5′ untranslated sequence is also advantageously employed. The 5′ untranslated sequence is the portion of an mRNA which extends from the 5′ cap site to the translation initiation codon. This region of the mRNA is necessary for translation initiation in plants and plays a role in the regulation of gene expression. Suitable 5′ untranslated regions for use in plants include those of Alfalfa mosaic virus, Cucumber mosaic virus coat protein gene, and Tobacco mosaic virus.

For efficient expression, the coding sequences are preferably also operatively linked to a 3′ untranslated sequence. The 3′ untranslated sequence will include a transcription termination sequence and a polyadenylation sequence. The 3′ untranslated region can be obtained from the flanking regions of genes from Agrobacterium, plant viruses, plants or other eukaryotes. Suitable 3′ untranslated sequences for use in plants include those of the Cauliflower mosaic virus 35S gene, the phaseolin seed storage protein gene, the pea ribulose bisphosphate carboxylase small subunit E9 gene, the soybean 7S storage protein genes, the octopine synthase gene, and the nopaline synthase gene.

The chimeric insecticidal toxin coding sequence described herein is advantageously expressed in the phloem of the plant. It is then consumed by a sap-sucking insect, in which transmission of a relevant virus from plant to plant is inhibited or prevented. The CaMV 35C promoter is a useful promoter for phloem expression.

Chimeric DNA construct(s) (non-naturally occurring nucleic acid molecules) described herein may contain multiple copies of a promoter or multiple copies of the peptide coding sequence of the present invention. In addition, the construct(s) may include coding sequences for selectable or detectable markers, each in proper reading frame with the other functional elements in the DNA molecule. The preparation of such constructs is within the ordinary level of skill in the art.

The DNA construct may be a vector. The vector may contain one or more replication systems which allow it to replicate in host cells. Self-replicating vectors include plasmids, cosmids and viral vectors. Alternatively, the vector may be an integrating vector which allows the integration into the host cell's chromosome of the DNA sequence encoding the chimeric insecticidal toxin described herein. The vector desirably also has unique restriction sites for the insertion of DNA sequences. If a vector does not have unique restriction sites, it may be modified to introduce or eliminate restriction sites to make it more suitable for further manipulations.

The DNA constructs herein can be used to transform any type of plant cells (see below). A genetic marker can be used for selecting transformed plant cells (a selection marker). Selection markers typically allow transformed cells to be recovered by negative selection (i.e., inhibiting growth of cells that do not contain the selection marker) or by screening for a product encoded by the selection marker.

A commonly used selectable marker gene for plant transformation is the neomycin phosphotransferase II (nptII) gene, isolated from Tn5, which, when placed under the control of plant expression control signals, confers resistance to kanamycin (Fraley et al. (1983) Proc. Natl. Acad. Sci. USA 80:4803). Another commonly used selectable marker gene is the hygromycin phosphotransferase gene which confers resistance to the antibiotic hygromycin. Vanden Elzen et al. (1995) Plant Mol. Biol. 5:299). Additional selectable marker genes of bacterial origin that confer resistance to antibiotics include gentamicin acetyl transferase, streptomycin phosphotransferase, aminoglycoside-3′-adenyl transferase, the phosphinothricin acetyltransferase conferring resistance to the herbicide phosphinothricin, and the bleomycin resistance determinant (Hayford et al. (1988) Plant Physiol. 86:1216; Jones et al. (1987). Mol. Gen. Genet. 210:86; Svab et al. (1990) Plant Mol. Biol. 14:197; Hille et al. (1986) Plant Mol. Biol. 7:171). Other selectable marker genes confer resistance to herbicides such as glyphosate, glufosinate or bromoxynil (Comai et al. (1985) Nature 317:741-744; Stalker et al. (1988) Science 242:419-423; Hinchee et al. (1988) Bio/Technology 6:915-922; Stalker et al. (1988) J. Biol. Chem. 263:6310-6314; Gordon-Kamm et al. (1990) Plant Cell 2:603-618).

Other selectable markers useful for plant transformation include, without limitation, mouse dihydrofolate reductase, plant 5-enolpyruvylshikimate-3-phosphate synthase, and plant acetolactate synthase (Eichholtz et al. (1987) Somatic Cell Mol. Genet. 13:67; Shah et al. (1986) Science 233:478; Charest et al. (1990) Plant Cell Rep. 8:643; EP 154,204.

Commonly used (reporter) genes for screening presumptively transformed cells include but are not limited to β-glucuronidase (GUS), β-galactosidase, luciferase, and chloramphenicol acetyltransferase (Jefferson, R. A. (1987) Plant Mol. Biol. Rep. 5:387; Teeri et al. (1989) EMBO J. 8:343; Koncz et al. (1987) Proc. Natl. Acad. Sci. USA 84:131; De Block et al. (1984) EMBO J. 3:1681), green fluorescent protein (GFP) (Chalfie et al. (1994) Science 263:802; Haseloff et al. (1995) TIG 11:328-329 and PCT application WO 97/41228). Another approach to the identification of relatively rare transformation events has been use of a gene that encodes a dominant constitutive regulator of the Zea mays anthocyanin pigmentation pathway (Ludwig et al. 1990. Science 247:449). To select cells which have successfully undergone transformation, it is preferred to introduce a selectable marker which confers, to the cells which have successfully undergone transformation, a resistance to a biocide (for example a herbicide), a metabolism inhibitor such as 2-deoxyglucose-6-phosphate (WO 98/45456) or an antibiotic. The selection marker permits the transformed cells to be selected from untransformed cells (McCormick et al. (1986) Plant Cell Reports 5:81-84). Suitable selection markers are described above and include antibiotic resistance markers, among others.

Numerous transformation vectors are available for plant transformation, and sequences encoding the chimeric insecticidal toxins described herein can be used in conjunction with any such vectors. The selection of vector for use will depend upon the preferred transformation technique and the target species for transformation. For certain target species, different antibiotic or herbicide selection markers may be preferred. Selectable markers used routinely in transformation include the nptII gene which confers resistance to kanamycin and related antibiotics (Messing and Vierra. (1982) Gene 19: 259-268; Bevan et al. (1983). Nature 304:184-187), the bar gene which confers resistance to the herbicide phosphinothricin (White et al. (1990) Nucl Acids Res 18: 1062; Spencer et al. (1990) Theor Appl Genet. 79: 625-631), the hph gene which confers resistance to the antibiotic hygromycin (Blochinger and Diggelmann. (1984) Mol Cell Biol 4: 2929-2931), and the dhfr gene, which confers resistance to methotrexate (Bourouis et al. (1983). EMBO J. 2(7): 1099-1104).

Many vectors are available for transformation using A. tumefaciens. These typically carry at least one T-DNA border sequence and include vectors such as pBIN19 (Bevan. 1984. Nucl. Acids Res.). Below the construction of two typical vectors is described. pCAMBIA and other vectors are well known to the art as well.

The exemplary binary vector pCIB10 contains a gene encoding kanamycin resistance for selection in plants, T-DNA right and left border sequences and incorporates sequences from the wide host-range plasmid pRK252 allowing it to replicate in both E. coli and Agrobacterium. Its construction is described by Rothstein et al. 1987. Gene 53: 153-161. Various derivatives of pCIB10 have been constructed which incorporate the gene for hygromycin B phosphotransferase described by Gritz et al. (1983). Gene 25: 179-188). These derivatives enable selection of transgenic plant cells on hygromycin only (pCIB743), or hygromycin and kanamycin (pCIB715, pCIB717). See, e.g., Rogers et al., Methods for Plant Molecular Biology, Weissbach and Weissbach, eds, Academic Press, San Diego, Calif., 1988, for a description of a kanamycin resistance marker. Other selective agents for use in plants include bleomycin, gentamicin and certain herbicide resistance markers.

Transformation without the use of A. tumefaciens circumvents the requirement for T-DNA sequences in the chosen transformation vector and consequently vectors lacking these sequences can be utilized in addition to vectors such as the ones described above which contain T-DNA sequences. Transformation techniques which do not rely on Agrobacterium include transformation via particle bombardment, protoplast uptake (e.g., PEG and electroporation) and microinjection. The choice of vector depends largely on the preferred selection for the species being transformed.

Gene sequences intended for expression in transgenic plants are first assembled in expression cassettes behind a suitable promoter and upstream of a suitable transcription terminator. These expression cassettes can then be easily transferred to the plant transformation vectors of choice.

A variety of transcriptional terminators are available for use in expression cassettes. These are responsible for the termination of transcription beyond the transgene and its correct polyadenylation. Appropriate transcriptional terminators and those which are known to function in plants and include the CaMV 35S terminator, the tml terminator, the nopaline synthase (nos) terminator, the pea rbcS E9 terminator. These can be used in both monocotyledonous and dicotyledonous plants.

Numerous sequences have been found to enhance gene expression from within the transcriptional unit and these sequences can be used in conjunction with the genes of this invention to increase their expression in transgenic plants.

Various intron sequences have been shown to enhance expression, particularly in monocotyledonous cells. For example, the introns of the maize Adh1 gene significantly enhance the expression of the wild-type gene under its cognate promoter when introduced into maize cells. Intron 1 enhances expression in fusion constructs with the chloramphenicol acetyltransferase gene (Callis et al. (1987) Genes Develop. 1: 1183-1200). In the same experimental system, the intron from the maize bronzel gene had a similar effect in enhancing expression. Intron sequences have been routinely incorporated into plant transformation vectors, typically within the non-translated leader.

A number of non-translated leader sequences derived from viruses also enhance expression, especially in dicotyledonous cells. Leader sequences from Tobacco mosaic virus (TMV, the “W-sequence”), Maize chlorotic mottle virus (MCMV), and Alfalfa mosaic virus (AMV) have been shown to enhance expression (e.g., Gallie et al. (1987) Nucl. Acids Res. 15: 8693-8711; Skuzeski et al. (1990) Plant Molec. Biol. 15:65-79).

Agrobacterium-mediated transformation is one technique for transformation of dicots because of the high efficiency of transformation and success with many different species. The many crop species which are routinely transformable by Agrobacterium include tobacco, tomato, sunflower, cotton, oilseed rape, potato, soybean, peas, beans, alfalfa and poplar (EP 317 511, cotton; EP 0 249 432, tomato, to Calgene; WO 87/07299, Brassica, to Calgene; U.S. Pat. No. 4,795,855, poplar). Agrobacterium transformation typically involves the transfer of the binary vector carrying the foreign DNA of interest to an appropriate Agrobacterium strain which may depend of the complement of vir genes carried by the host Agrobacterium strain either on a co-resident Ti plasmid or chromosomally (e.g., strain CIB542 for pCIB200 and pCIB2001 (Uknes et al. (1993) Plant Cell 5: 159-169). The transfer of the recombinant binary vector to Agrobacterium is accomplished by a triparental mating procedure using E. coli carrying the recombinant binary vector, a helper E. coli strain which carries a plasmid such as pRK2013 and which is able to mobilize the recombinant binary vector to the target Agrobacterium strain. Alternatively, the recombinant binary vector can be transferred to Agrobacterium by DNA transformation (Hofgen and Willmitzer. (1988) Nucl. Acids Res. 16: 9877).

The following include representative publications disclosing protocols that can be used to genetically transform various plant species including rice (Alam et al., 1999, Plant Cell Rep. 18, 572); apple (Yao et al., 1995, Plant Cell Reports 14, 407-412); maize (U.S. Pat. Nos. 5,177,010 and 5,981,840); wheat (Ortiz et al., 1996, Plant Cell Rep. 15, 1996, 877); tomato (U.S. Pat. No. 5,159,135); potato (Kumar et al., 1996 Plant J. 9, : 821); cassaya (Li et al., 1996 Nat. Biotechnology 14, 736); lettuce (Michelmore et al., 1987, Plant Cell Rep. 6, 439); tobacco (Horsch et al., 1985, Science 227, 1229); cotton (U.S. Pat. Nos. 5,846,797 and 5,004,863); grasses (U.S. Pat. Nos. 5,187,073 and 6,020,539); peppermint (Niu et al., 1998, Plant Cell Rep. 17, 165); citrus plants (Pena et al., 1995, Plant Sci. 104, 183); caraway (Krens et al., 1997, Plant Cell Rep, 17, 39); banana (U.S. Pat. No. 5,792,935); soybean (U.S. Pat. Nos. 5,416,011; 5,569,834; 5,824,877; 5,563,04455 and 5,968,830); pineapple (U.S. Pat. No. 5,952,543); poplar (U.S. Pat. No. 4,795,855); monocots in general (U.S. Pat. Nos. 5,591,616 and 6,037,522); brassica (U.S. Pat. Nos. 5,188,958; 5,463,174 and 5,750,871); cereals (U.S. Pat. No. 6,074,877); pear (Matsuda et al., 2005, Plant Cell Rep. 24(1):45-51); Prunus (Ramesh et al., 2006 Plant Cell Rep. 25(8):821-8; Song and Sink 2005 Plant Cell Rep. 2006; 25(2):117-23; Gonzalez Padilla et al., 2003 Plant Cell Rep. 22(1):38-45); strawberry (Oosumi et al., 2006 Planta. 223(6):1219-30; Folta et al., 2006 Planta April 14; PMID: 16614818), rose (Li et al., 2003), Rubus (Graham et al., 1995 Methods Mol. Biol. 1995; 44:129-33), tomato (Dan et al., 2006, Plant Cell Reports V25:432-441), apple (Yao et al., 1995, Plant Cell Rep. 14, 407-412) and Actinidia eriantha (Wang et al., 2006, Plant Cell Rep. 25,5: 425-31). Transformation of other species is also contemplated. Suitable methods and protocols are available in the scientific literature.

Once an expression construct or expression vector of the invention has been established, it can be transformed into a plant cell. A variety of methods for introducing nucleic acid sequences (e.g., vectors) into the genome of plants and for the regeneration of plants from plant tissues or plant cells are known (Plant Molecular Biology and Biotechnology (CRC Press, Boca Raton, Fla., pp. 71-119 (1993); White F F. (1993) Vectors for Gene Transfer in Higher Plants; in: Transgenic Plants, vol. 1, Engineering and Utilization, Ed.: Kung and Wu R, Academic Press, 15-38; Jenes et al. (1993) Techniques for Gene Transfer, in: Transgenic Plants, vol. 1, Engineering and Utilization, Ed.: Kung and R. Wu, Academic Press, pp. 128-143; Potrykus et al. (1991) Annu. Rev. Plant Physiol. Plant Molec. Biol. 42:205-225; Halford and Shewry. 2000. Br. Med. Bull. 56:62-73).

Transformation methods may include direct and indirect methods of transformation. Suitable direct methods include polyethylene glycol induced DNA uptake, liposome-mediated transformation (U.S. Pat. No. 4,536,475), biolistic methods using the gene gun (particle bombardment; Fromm et al. (1990) Bio/Technology. 8:833-9; Gordon-Kamm et al. (1990) Plant Cell 2:603), electroporation, incubation of dry embryos in DNA-comprising solution, and microinjection. In the case of these direct transformation methods, the plasmid used need not meet any particular requirements. Simple plasmids, such as those of the pUC series, pBR322, M13 mp series, pACYC184 and the like can be used. If intact plants are to be regenerated from the transformed cells, an additional selectable marker gene is preferably located on the plasmid. The direct transformation techniques are equally suitable for dicotyledonous and monocotyledonous plants.

Transformation can also be carried out by bacterial infection by means of Agrobacterium (for example EP 116,718), viral infection by means of viral vectors (EP 067,553; U.S. Pat. No. 4,407,956; WO 95/34668; WO 93/03161) or by means of pollen (EP 270,356; WO 85/01856; U.S. Pat. No. 4,684,611). Agrobacterium based transformation techniques (especially for dicotyledonous plants) are well known in the art. The Agrobacterium strain (e.g., Agrobacterium tumefaciens or Agrobacterium rhizogenes) comprises a plasmid (Ti or Ri plasmid) and a T-DNA element which is transferred to the plant following infection with Agrobacterium. The T-DNA (transferred DNA) is integrated into the genome of the plant cell. The T-DNA may be localized on the Ri- or Ti-plasmid or is separately comprised in a so-called binary vector. Methods for the Agrobacterium-mediated transformation are described, for example, in Horsch R B et al. (1985) Science 225:1229f. The Agrobacterium-mediated transformation is best suited to dicotyledonous plants but has also been adapted to monocotyledonous plants. The transformation of plants by Agrobacteria is described, for example, in White FF, Vectors for Gene Transfer in Higher Plants; in Transgenic Plants, Vol. 1, Engineering and Utilization, edited by S. D. Kung and R. Wu, Academic Press, (1993), pp. 15-38; Jenes B et al. (1993) Techniques for Gene Transfer, in: Transgenic Plants, Vol. 1, Engineering and Utilization, edited by S. D. Kung and R. Wu, Academic Press, pp. 128-143; Potrykus (1991). Annu Rev Plant Physiol Plant Molec Biol 42:205-225).

Transformation may result in transient or stable transformation and expression; stable transformation is preferred in the cells, plants, and methods herein. Although a sequence encoding a chimeric insecticidal toxin can be inserted into any plant and plant cell, it is particularly useful in crop plant cells (including those of vegetables, grains, fruits and other agricultural plants for human or animal use) as well as in horticultural plant cells, such as ornamental or ground cover plant cells.

Various tissues are suitable as starting material (explant) for the Agrobacterium-mediated transformation process including but not limited to callus (U.S. Pat. No. 5,591,616; EP 604 662), immature embryos (EP 672 752), pollen (U.S. Pat. No. 5,929,300), shoot apex (U.S. Pat. No. 5,164,310), or in planta transformation (U.S. Pat. No. 5,994,624). The method and material described herein can be combined with virtually all Agrobacterium mediated transformation methods known in the art. Preferred combinations include, but are not limited, to the following starting materials and methods: monocotyledonous plants: EP-A1 672 752, EP-A1 604 662, U.S. Pat. No. 6,074,877, U.S. Pat. No. 6,037,522, WO 01/12828; banana, U.S. Pat. No. 5,792,935; EP 731 632; U.S. Pat. No. 6,133,035; barley, WO 99/04618; maize, U.S. Pat. No. 5,177,010; U.S. Pat. No. 5,987,840; pineapple, U.S. Pat. No. 5,952,543; WO 01/33943; soybean, U.S. Pat. No. 5,376,543; EP 397 687; U.S. Pat. No. 5,416,011; U.S. Pat. No. 5,968,830; U.S. Pat. No. 5,563,055; U.S. Pat. No. 5,959,179; EP 652 965; EP 1,141,346; brassicacious plants, U.S. Pat. No. 5,188,958; EP 270 615; EP-A1 1,009,845; beans, U.S. Pat. No. 5,169,770; EP 397 687; peas, U.S. Pat. No. 5,286,635; cotton, U.S. Pat. No. 5,004,863; EP-A1 270 355; U.S. Pat. No. 5,846,797; EP-A1 1,183,377; EP-A1 1,050,334; EP-A1 1,197,579; EP-A1 1,159,436, U.S. Pat. No. 5,929,300, U.S. Pat. No. 5,994,624, and tomato, U.S. Pat. No. 5,565,347, and other plants and methods are also known to the art.

Transformation of most monocotyledon species has now also become routine. Preferred techniques include direct gene transfer into protoplasts using PEG or electroporation techniques, and particle bombardment into callus tissue. Transformations can be undertaken with a single DNA species or multiple DNA species (i.e., co-transformation) and both these techniques are suitable for use with this invention. Co-transformation may have the advantage of avoiding complex vector construction and of generating transgenic plants with unlinked loci for the gene of interest and the selectable marker, enabling the removal of the selectable marker in subsequent generations, should this be regarded desirable. However, a disadvantage of the use of co-transformation is the less than 100% frequency with which separate DNA species are integrated into the genome (Schocher et al. (1986) Biotechnology 4:1093-1096). EP 0 292 435, EP 0 392 225 and WO 93107278 describe techniques for the preparation of callus and protoplasts from an elite inbred line of maize, transformation of protoplasts using PEG or electroporation, and the regeneration of maize plants from transformed protoplasts. Gordon-Kamm et al. (1990) Plant Cell 2: 603-618 and Fromm et al. (1990) Biotechnology 8: 833-839 have published techniques for transformation of A188-derived maize line using particle bombardment. Furthermore, WO 93/07278 and Koziel et al. (1993) Biotechnology 11: 194-200 describe techniques for the transformation of elite inbred lines of maize by particle bombardment. This technique utilizes immature maize embryos of 1.5-2.5 mm length excised from a maize ear 14-15 days after pollination and a biolistics device for bombardment.

Transformation of rice can also be undertaken by direct gene transfer techniques utilizing protoplasts or particle bombardment. Protoplast-mediated transformation has been described for Japonica-types and Indica-types (Zhang et al. (1988) Plant Cell Rep 7:379-384; Shimamoto et al. (1989) Nature 338: 274-277; Datta et al. (1990) Biotechnology 8: 736-740). Both types are also routinely transformable using particle bombardment (Christou et al. (1991) Biotechnology 9: 957-962).

Transgenic plants can be regenerated in the known manner from the transformed cells. The resulting plantlets can be planted and grown in the customary manner. Preferably, two or more generations should be cultured to ensure that the genomic integration is stable and hereditary. Suitable methods are described (Fennell et al. (1992) Plant Cell Rep. 11: 567-570; Stoeger et al. (1995) Plant Cell Rep. 14:273-278; Jahne et al. (1994) Theor Appl Genet. 89:525-533).

EP 332 581 describes techniques for the generation, transformation and regeneration of Pooideae protoplasts. These techniques allow the transformation of Dactylis and wheat. Furthermore, wheat transformation was been described by Vasil et al. (1992) Biotechnology 10: 667-674) using particle bombardment into cells of type C long-term regenerable callus, and also by Vasil et al. (1993) Biotechnology 11: 1553-1558 and Weeks et al. (1993) Plant Physiol. 102: 1077-1084 using particle bombardment of immature embryos and immature embryo-derived callus. A preferred technique for wheat transformation, however, involves the transformation of wheat by particle bombardment of immature embryos and includes either a high sucrose or a high maltose step prior to gene delivery. Prior to bombardment, any number of embryos (0.75-1 mm in length) are plated onto MS medium with 3% sucrose (Murashige and Skoog. (1962) Physiologia Plantarum 15: 473497) and 3 mg/l 2,4-D for induction of somatic embryos which is allowed to proceed in the dark. On the chosen day of bombardment, embryos are removed from the induction medium and placed onto the osmoticum (i.e., induction medium with sucrose or maltose added at the desired concentration, typically 15%). The embryos are allowed to plasmolyze for 2-3 h and are then bombarded. Twenty embryos per target plate is typical, although not critical. An appropriate gene-carrying plasmid (such as pCIB3064 or pSG35) is precipitated onto micrometer size gold particles using standard procedures. Each plate of embryos is shot with the DuPont Biolistics, helium device using a burst pressure of about 1000 psi using a standard 80 mesh screen. After bombardment, the embryos are placed back into the dark to recover for about 24 h (still on osmoticum). After 24 hrs, the embryos are removed from the osmoticum and placed back onto induction medium where they stay for about a month before regeneration. Approximately one month later the embryo explants with developing embryogenic callus are transferred to regeneration medium (MS+1 mg/liter NAA, 5 mg/liter GA), further containing the appropriate selection agent (10 mg/l basta in the case of pCIB3064 and 2 mg/l methotrexate in the case of pSOG35). After approximately one month, developed shoots are transferred to larger sterile containers known as “GA7s” which contained half-strength MS, 2% sucrose, and the same concentration of selection agent. U.S. patent application Ser. No. 08/147,161 describes methods for wheat transformation.

Expression directed by a particular sequence means there is transcription and translation of an associated downstream sequence. With reference to tissue-specific regulation of expression of a peptide sequence of interest operably linked to the plant-expressible transcription regulatory sequence, expression may be advantageously determined by a strong constitutive promoter such as the Cauliflower Mosaic Virus 19S or 35 S promoter, a tandem repeat 35S promoter, the actin 2 promoter from Arabidopsis thaliana, among others, or advantageously, a phloem-specific promoter.

Transformation to provide transgenic plants expressing a peptide of the invention can be carried out by art-known methods. A vector construct carrying a nucleotide sequence encoding a peptide of the invention such as PhD3.1 (SEQ ID NO:9) will optionally include DNA encoding a signal peptide to provide for export of the peptide from the transformed cell and a suitable plant promoter. A phloem-specific promoter is preferred, allowing expression to be maximized in phloem tissue (see, e.g., Booker, J. et al. (2003) Plant Cell 15(2):495-507; Jones, J. D. et al. (1992) Transgenic Res. 1(6):285-297; Truenit, E. et al. (1995) Planta 196(3):564-570). However, a highly active promoter, such as CaMV 35S promoter can also be used. Given the small size of peptides of the invention, the expression level can be increased by including multiple copies of the same peptide controlled by a single promoter (see, e.g., Marcos, J. F. et al. (1994) Plant Mol. Biol. 24:495-503; Beck von Bodman, S. et al. (1995) Bio/technology 13:587-591).

Transformation can be carried out by a variety of known methods. Commercial facilities for carrying out plant transformation are available, e.g., at the Iowa State University, Plant Transformation Facility, Ames Iowa, and techniques for transformation are well known and widely accessible in the art. Suitable transformants are identified or selected by means known in the art. Those skilled in the art can make appropriate choices from known methods transformation, selection and regeneration based on the plant species to be transformed. The choice of plant species will be determined by the virus whose inhibition is desired.

Selected transformants are regenerated using art-known methods appropriate for the desired plant species. For pea (Pisum sativum) see, e.g., Nauerby, B. et al. (1991) Plant Cell Reports 676-679.

The plants transformed to contain and express a chimeric aphicidal or other insecticidal toxin as described herein may be grown and either selfed or crossed with a different plant strain and the resulting hybrids, with the desired phenotypic characteristics, may be identified. Two or more generations may be grown to ensure that the subject phenotypic characteristics are stably maintained and inherited. Plants resulting from such standard breeding approaches also within the scope of the present disclosure.

All references and patent documents cited herein reflect the level of skill in the relevant arts and are incorporated by reference in their entireties to the extent there is no inconsistency with the present disclosure.

The examples provided herein are for illustrative purposes and are not intended to limit the scope of the invention as claimed. Any variations in the exemplified compositions, plants and methods which occur to the skilled artisan are intended to fall within the scope of the present invention.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner. Encompassed within comprising are consisting essentially of and consisting of:

Monoclonal or polyclonal antibodies specifically reacting with a chimeric insecticidal protein of interest can be made by methods well known in the art. See, e.g., Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Wiley Interscience/Greene Publishing, New York, N.Y.

As used interchangeably herein, the terms “nucleic acid molecule(s)”, “oligonucleotide(s)”, and “polynucleotide(s)” include RNA or DNA (either single or double stranded, coding, complementary or antisense), or RNA/DNA hybrid sequences of more than one nucleotide in either single chain or duplex form (although each of the above species may be particularly specified). The term “nucleotide” is used herein as an adjective to describe molecules comprising RNA, DNA, or RNA/DNA hybrid sequences of any length in single-stranded or duplex form. More precisely, the expression “nucleotide sequence” encompasses the nucleic material itself and is thus not restricted to the sequence information (e.g. the succession of letters chosen among the four base letters) that biochemically characterizes a specific DNA or RNA molecule. The term “nucleotide” is also used herein as a noun to refer to individual nucleotides or varieties of nucleotides, meaning a molecule, or individual unit in a larger nucleic acid molecule, comprising a purine or pyrimidine, a ribose or deoxyribose sugar moiety, and a phosphate group, or phosphodiester linkage in the case of nucleotides within an oligonucleotide or polynucleotide. The term “nucleotide” is also used herein to encompass “modified nucleotides” which comprise at least one modifications such as (a) an alternative linking group, (b) an analogous form of purine, (c) an analogous form of pyrimidine, or (d) an analogous sugar. For examples of analogous linking groups, purine, pyrimidines, and sugars see for example, WO 95/04064, which disclosure is hereby incorporated by reference in its entirety. Preferred modifications of the present invention include, but are not limited to, 5-fluorouracil, 5-bromouracil, 5-chlorouracil, 5-iodouracil, hypoxanthine, xanthine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylguanosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylguanosine, 5′-methoxycarboxymethyluracil, 5-methoxyuracil, 2-methylthio-N6-isopentenyladenine, uracil-5-oxyacetic acid (v) ybutoxosine, pseudouracil, guanosine, 2-thiocytosine, 5-methyl-2-thiouracil, 2-thiouracil, 4-thiouracil, 5-methyluracil, uracil-5-oxyacetic acid methylester, uracil-5-oxyacetic acid, 5-methyl-2-thiouracil, 3-(3-amino-3-N2-carboxypropyl)uracil, and 2,6-diaminopurine. The polynucleotide sequences herein may be prepared by any known method, including synthetic, recombinant, ex vivo generation, or a combination thereof, as well as utilizing any purification methods known in the art. Methylenemethylimino linked oligonucleotides as well as mixed backbone compounds, may be prepared as described in U.S. Pat. Nos. 5,378,825; 5,386,023; 5,489,677; 5,602,240; and 5,610,289. Formacetal and thioformacetal linked oligonucleotides may be prepared as described in U.S. Pat. Nos. 5,264,562 and 5,264,564. Ethylene oxide linked oligonucleotides may be prepared as described in U.S. Pat. No. 5,223,618. Phosphinate oligonucleotides may be prepared as described in U.S. Pat. No. 5,508,270. Alkyl phosphonate oligonucleotides may be prepared as described in U.S. Pat. No. 4,469,863. 3′-Deoxy-3′-methylene phosphonate oligonucleotides may be prepared as described in U.S. Pat. No. 5,610,289 or 5,625,050. Phosphoramidite oligonucleotides may be prepared as described in U.S. Pat. No. 5,256,775 or 5,366,878. Alkylphosphonothioate oligonucleotides may be prepared as described in WO 94/17093 and WO 94/02499. 3′-Deoxy-3′-amino phosphoramidate oligonucleotides may be prepared as described in U.S. Pat. No. 5,476,925. Phosphotriester oligonucleotides may be prepared as described in U.S. Pat. No. 5,023,243. Borano phosphate oligonucleotides may be prepared as described in U.S. Pat. Nos. 5,130,302 and 5,177,198.

The term “upstream” is used herein to refer to a location which is toward the 5′ end of the polynucleotide from a specific reference point.

The terms “base paired” and “Watson & Crick base paired” are used interchangeably herein to refer to nucleotides which can be hydrogen bonded to one another by virtue of their sequence identities in a manner like that found in double-helical DNA with thymine or uracil residues linked to adenine residues by two hydrogen bonds and cytosine and guanine residues linked by three hydrogen bonds.

The terms “complementary” or “complement thereof” are used herein to refer to the sequences of polynucleotides which is capable of forming Watson & Crick base pairing with another specified polynucleotide throughout the entirety of the complementary region. For the purpose of the present invention, a first polynucleotide is deemed to be complementary to a second polynucleotide when each base in the first polynucleotide is paired with its complementary base. Complementary bases are, generally, A and T (or A and U), or C and G. “Complement” is used herein as a synonym from “complementary polynucleotide”, “complementary nucleic acid” and “complementary nucleotide sequence”. These terms are applied to pairs of polynucleotides based solely upon their sequences and not any particular set of conditions under which the two polynucleotides would actually bind. Unless otherwise stated, all complementary polynucleotides are fully complementary on the whole length of the considered polynucleotide.

The terms “polypeptide” and “protein”, used interchangeably herein, refer to a polymer of amino acids without regard to the length of the polymer; thus, peptides, oligopeptides, and proteins are included within the definition of polypeptide. This term also does not specify or exclude chemical or post-expression modifications of the polypeptides herein, although chemical or post-expression modifications of these polypeptides may be included excluded as specific embodiments. Therefore, for example, modifications to polypeptides that include the covalent attachment of glycosyl groups, acetyl groups, phosphate groups, lipid groups and the like are expressly encompassed by the term polypeptide. Further, polypeptides with these modifications may be specified as individual species to be included or excluded from the present invention. The natural or other chemical modifications, such as those listed in examples above can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristoylation, oxidation, pegylation, proteolytic processing, phosphorylation, prenylation, racemization, selenoylation, sulfation, transfer-RNA mediated addition of amino acids to proteins such as arginylation, and ubiquitination, as known to the art. Also included within the definition are polypeptides which contain one or more analogs of an amino acid (including, for example, non-naturally occurring amino acids, amino acids which only occur naturally in an unrelated biological system, modified amino acids from mammalian systems, etc.), polypeptides with substituted linkages, as well as other modifications known in the art, both naturally occurring and non-naturally occurring.

As used herein, the terms “recombinant polynucleotide” and “polynucleotide construct” are used interchangeably to refer to linear or circular, purified or isolated polynucleotides that have been artificially designed and which comprise at least two nucleotide sequences that are not found as contiguous nucleotide sequences in their initial natural environment. In particular, these terms mean that the polynucleotide or cDNA is adjacent to “backbone” nucleic acid to which it is not adjacent in its natural environment. Additionally, to be “enriched” the cDNAs will represent 5% or more of the number of nucleic acid inserts in a population of nucleic acid backbone molecules. Backbone molecules according to the present invention include nucleic acids such as expression vectors, self-replicating nucleic acids, viruses, integrating nucleic acids, and other vectors or nucleic acids used to maintain or manipulate a nucleic acid insert of interest. Preferably, the enriched cDNAs represent 15% or more of the number of nucleic acid inserts in the population of recombinant backbone molecules. More preferably, the enriched cDNAs represent 50% or more of the number of nucleic acid inserts in the population of recombinant backbone molecules. In a highly preferred embodiment, the enriched cDNAs represent 90% or more (including any number between 90 and 100%, to the thousandth position, e.g., 99.5%) of the number of nucleic acid inserts in the population of recombinant backbone molecules.

As used herein, the term “operably linked” refers to a linkage of polynucleotide elements in a functional relationship. A sequence which is “operably linked” to a regulatory sequence such as a promoter means that said regulatory element is in the correct location and orientation in relation to the nucleic acid to control RNA polymerase initiation and expression of the nucleic acid of interest. For instance, a promoter or enhancer is operably linked to a coding sequence if it affects the transcription of the coding sequence.

In certain embodiments, an expression construct or expression vector, any type of genetic construct containing a nucleic acid coding for a gene product in which part or all of the nucleic acid coding sequence is capable of being transcribed, is constructed so that the coding sequence of interest is operably linked to and is expressed under transcriptional control of a promoter. A “promoter” refers to a DNA sequence recognized by the synthetic machinery of the cell, or introduced synthetic machinery, required to initiate the specific transcription of a gene. The phrase “under transcriptional control” can mean that the promoter is in the correct location and orientation in relation to the nucleic acid to control RNA polymerase initiation and expression of the gene in the isolated host cell of interest.

Where a cDNA insert is employed, typically one can include a polyadenylation signal to effect proper polyadenylation of the gene transcript. A terminator is also contemplated as an element of the expression construct. These elements can serve to enhance message levels and to minimize read through from the construct into other sequences.

In certain embodiments, the expression construct or vector contains a reporter gene whose activity may be detected or measured to determine the effect of a bi-directional, host-factor independent transcriptional terminators element or other element. Conveniently, the reporter gene produces a product that is easily assayed, such as a colored product, a fluorescent product or a luminescent product. Many examples of reporter genes are available, such as the genes encoding GFP (green fluorescent protein), CAT (chloramphenicol acetyltransferase), luciferase, GAL (β-galactosidase), GUS (β-glucuronidase), etc. The particular reporter gene employed is not important, provided it is capable of being expressed and expression can be detected. Further examples of reporter genes are well known to the art, and any of those known may be used in the practice of the claimed methods.

The term “chimeric” with reference to polypeptides encompasses recombinantly and synthetically produced polypeptides containing portions from different sources. Variant polypeptide sequences preferably exhibit at least 75%, more preferably at least 76%, more preferably at least 77%, more preferably at least 78%, more preferably at least 79%, more preferably at least 80%, more preferably at least 81%, more preferably at least 82%, more preferably at least 83%, more preferably at least 84%, more preferably at least 85%, more preferably at least 86%, more preferably at least 87%, more preferably at least 88%, more preferably at least 89%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, and most preferably at least 99% identity to a specifically exemplified sequence disclosed herein. Identity is found over a comparison window of at least 20 amino acid positions, preferably at least 50 amino acid positions, more preferably at least 100 amino acid positions, and most preferably over the entire length of an insecticidal polypeptide.

Polypeptide sequence identity can be determined in the following manner. The subject polypeptide sequence is compared to a candidate polypeptide sequence using BLASTP (from the BLAST suite of programs, version 2.2.5 [Nov. 2002]) in bl2seq, which is publicly available via the NCBI website. The default parameters of bl2seq are utilized except that filtering of low complexity regions should be turned off.

Polypeptide sequence identity may also be calculated over the entire length of the overlap between a candidate and subject polynucleotide sequences using global sequence alignment programs. EMBOSS-needle (available on the worldwide web, address ebi.ac.uk/emboss/align/, and GAP (Huang, X. (1994) On Global Sequence Alignment. Computer Applications in the Biosciences 10, 227-235.) as discussed above are also suitable global sequence alignment programs for calculating polypeptide sequence identity.

A preferred method for calculating polypeptide % sequence identity is based on aligning sequences to be compared using Clustal X (Jeanmougin et al., 1998, Trends Biochem. Sci. 23, 403-5).

Polypeptide variants herein or used in the methods herein, also encompass those which exhibit a similarity to one or more of the specifically identified sequences that is likely to preserve the functional equivalence of those sequences and which could not reasonably be expected to have occurred by random chance. Such sequence similarity with respect to polypeptides may be determined using the publicly available bl2seq program from the BLAST suite of programs (version 2.2.5 [Nov. 2002]) from the NCBI website. The similarity of polypeptide sequences may be examined using the following unix command line parameters:
bl2seq −i peptideseq1 −j peptideseq2 −F F −p blastp

Variant polypeptide sequences preferably exhibit an E value of less than 1×10-6 more preferably less than 1×10-9, more preferably less than 1×10-12, more preferably less than 1×10-15, more preferably less than 1×10-18, more preferably less than 1×10-21, more preferably less than 1×10-30, more preferably less than 1×10-40, more preferably less than 1×10-50, more preferably less than 1×10-60, more preferably less than 1×10-70, more preferably less than 1×10-80, more preferably less than 1×10-90 and most preferably 1×10-100 when compared with any one of the specifically identified sequences.

The parameter −F F turns off filtering of low complexity sections. The parameter −p selects the appropriate algorithm for the pair of sequences. This program finds regions of similarity between the sequences and for each such region reports an “E value” which is the expected number of times one could expect to see such a match by chance in a database of a fixed reference size containing random sequences. For small E values, much less than one, this is approximately the probability of such a random match.

Conservative substitutions of one or several amino acids of a described polypeptide sequence without significantly altering its biological activity are also included in the invention. A skilled artisan will be aware of methods for making phenotypically silent amino acid substitutions (see, e.g., Bowie et al., 1990, Science 247, 1306).

The term “genetic construct” refers to a polynucleotide molecule, usually double-stranded DNA, which may have inserted into it another polynucleotide molecule (the insert polynucleotide molecule) such as, but not limited to, a cDNA molecule. A genetic construct may contain the necessary elements that permit transcribing the insert polynucleotide molecule, and, optionally, translating the transcript into a polypeptide. The insert polynucleotide molecule may be derived from the host cell, or may be derived from a different cell or organism and/or may be a recombinant polynucleotide. Once inside the host cell, the genetic construct may become integrated in the host chromosomal DNA. The genetic construct may be linked to a vector.

The term “vector” refers to a polynucleotide molecule, usually double stranded DNA, which is used to transport the genetic construct into a host cell. The vector may be capable of replication in at least one additional host system, such as E. coli.

The term “expression construct” refers to a genetic construct that includes the necessary elements that permit transcribing the insert polynucleotide molecule, and, optionally, translating the transcript into a polypeptide. An expression construct typically comprises in a 5′ to 3′ direction:
a) a promoter functional in the host cell into which the construct will be transformed,
b) the polynucleotide to be expressed, and
c) a terminator functional in the host cell into which the construct will be transformed.

The term “coding region” or “open reading frame” (ORF) refers to the sense strand of a genomic DNA sequence or a cDNA sequence that is capable of producing a transcription product and/or a polypeptide under the control of appropriate regulatory sequences. The coding sequence is identified by the presence of a 5′ translation start codon and a 3′ translation stop codon. When inserted into a genetic construct, a “coding sequence” is capable of being expressed when it is operably linked to promoter and terminator sequences.

“Operably-linked” means that the sequenced to be expressed is placed under the control of regulatory elements that include promoters, tissue-specific regulatory elements, temporal regulatory elements, enhancers, repressors and terminators.

A plant expressible promoter is one which directs transcription of an associated nucleotide sequence in a plant cell. It may be tissue-specific (e.g., phloem-specific or leaf or root specific) or it may be expressed in response to an environmental signal such as wounding or light. Alternatively a plant expressible promoter may be constitutive, i.e., expressed in essentially all plant tissue.

A gut binding peptide (or multimer thereof) binds to the surface of the gut of an insect, especially the midgut. When incorporated into an insecticidal toxin (as part of an in-frame fusion) that does not normally bind and kill the insect, the gut binding peptide mediates the binding of the associated toxin to the gut and increases the toxicity of the protein to that insect.

General references for cloning include Maniatis et al., Molecular Cloning: A Laboratory Manual, Cold Spring Harbor, N.Y. (1982), Sambrook et al., Molecular Cloning: A Laboratory Manual, Cold Spring Harbor, N.Y. (1989); Ausubel 1993, Current Protocols in Molecular Biology, Wiley, NY.

Nucleotide Sequence Encoding Cyt2A (EU835185) (SEQ ID NO:27)

ATGTATACTAAAAATTTTAGTAATTCCAGAATGGAAGTAAAAGGTAATAACGGCTGTTCTGCAC
CTATTATTAGAAAACCATTTAAACATATTGTATTAACGGTTCCATCCAGTGATTTAGATAATTT
TAATACAGTCTTTTATGTACAACCACAATACATTAATCAGGCTCTTCATTTAGCAAATGCTTTT
CAAGGGGCTATAGACCCACTTAATTTAAATTTCAATTTTGAAAAGGCACTCCAAATTGCAAATG
GTATTCCTAATTCTGCAATTGTAAAAACTCTTAATCAAAGTGTTATACAGCAAACAGTTGAAAT
TTCAGTTATGGTTGAGCAACTTAAAAAGATTATTCAAGAGGTTTTAGGACTTGTTATTAACAGT
ACTAGTTTTTGGAATTCGGTAGAAGCTACAATTAAAGGCACATTTACAAATTTAGACACTCAAA
TAGATGAAGCATGGATTTTTTGGCATAGTTTATCCGCCCACAATACAAGTTATTATTATAATAT
TTTATTTTCTATTCAAAATGAAGATACAGGTGCAGTTATGGCAGTATTACCTTTAGCATTTGAG
GTTTCTGTGGATGTTGAAAAACAAAAAGTATTATTCTTTACAATAAAAGATAGTGCACGATATG
AAGTTAAAATGAAAGCTTTGACTTTAGTTCAAGCTCTACATTCCTCTGATGCCCCAATTGTAGA
TATATTTAATGTTAATAACTATAATTTATACCATTCTAATCATAAGATTATTCAAAATTTAAAT
TTATCGAATTGA

Amino acid sequence (ACF35049): (SEQ ID NO:28)

MYTKNFSNSRMEVKGNNGCSAPIIRKPFKHIVLTVPSSDLDNFNTVFYVQPQYINQALHLANAF
QGAIDPLNLNFNFEKALQIANGIPNSAIVKTLNQSVIQQTVEISVMVEQLKKIIQEVLGLVINS
TSFWNSVEATIKGTFTNLDTQIDEAWIFWHSLSAHNTSYYYNILFSIQNEDTGAVMAVLPLAFE
VSVDVEKQKVLFFTIKDSARYEVKMKALTLVQALHSSDAPIVDIFNVNNYNLYHSNHKIIQNLN
LSN

Nucleotide and amino acid sequence of Cyt2Aa-GBP3.1 and GBP3.1 mutants with aphicidal activity (Underlined sequences GBP3.1)

Nucleotide Sequence encoding CGAL1 (SEQ ID NO:29)

ATGTATACTAAAAATTTTAGTAATTCCAGAATGGAAGTAAAAGGTAATAACGGCTGTTCTGCAC
CTATTATTAGAAAACCATTTAAACATATTGTATTAACGGTTCCATCCAGTGATTTAGATAATTT
TACGTGTAGTAAGAAGTATCCGCGTTCTCCGTGTATGAATACAGTCTTTTATGTACAACCACAA
TACATTAATCAGGCTCTTCATTTAGCAAATGCTTTTCAAGGGGCTATAGACCCACTTAATTTAA
ATTTCAATTTTGAAAAGGCACTCCAAATTGCAAATGGTATTCCTAATTCTGCAATTGTAAAAAC
TCTTAATCAAAGTGTTATACAGCAAACAGTTGAAATTTCAGTTATGGTTGAGCAACTTAAAAAG
ATTATTCAAGAGGTTTTAGGACTTGTTATTAACAGTACTAGTTTTTGGAATTCGGTAGAAGCTA
CAATTAAAGGCACATTTACAAATTTAGACACTCAAATAGATGAAGCATGGATTTTTTGGCATAG
TTTATCCGCCCACAATACAAGTTATTATTATAATATTTTATTTTCTATTCAAAATGAAGATACA
GGTGCAGTTATGGCAGTATTACCTTTAGCATTTGAGGTTTCTGTGGATGTTGAAAAACAAAAAG
TATTATTCTTTACAATAAAAGATAGTGCACGATATGAAGTTAAAATGAAAGCTTTGACTTTAGT
TCAAGCTCTACATTCCTCTGATGCCCCAATTGTAGATATATTTAATGTTAATAACTATAATTTA
TACCATTCTAATCATAAGATTATTCAAAATTTAAATTTATCGAATTGA

Amino acid sequence of CGAL1 (SEQ ID NO:30)

MYTKNFSNSRMEVKGNNGCSAPIIRKPFKHIVLTVPSSDLDNFTCSKKYPRSPCMNTVFYVQPQ
YINQALHLANAFQGAIDPLNLNFNFEKALQIANGIPNSAIVKTLNQSVIQQTVEISVMVEQLKK
IIQEVLGLVINSTSFWNSVEATIKGTFTNLDTQIDEAWIFWHSLSAHNTSYYYNILFSIQNEDT
GAVMAVLPLAFEVSVDVEKQKVLFFTIKDSARYEVKMKALTLVQALHSSDAPIVDIFNVNNYNL
YHSNHKIIQNLNLSN

Nucleotide Sequence encoding CGAL3 (SEQ ID NO:31)

ATGTATACTAAAAATTTTAGTAATTCCAGAATGGAAGTAAAAGGTAATAACGGCTGTTCTGCAC
CTATTATTAGAAAACCATTTAAACATATTGTATTAACGGTTCCATCCAGTGATTTAGATAATTT
TAATACAGTCTTTTATGTACAACCACAATACATTAATCAGGCTCTTCATTTAGCAAATGCTTTT
CAAGGGGCTATAGACCCACTTAATTTAAATTTCAATTTTGAAAAGGCACTCCAAATTGCAAATG
GTATTCCTAATTCTGCAATTGTAAAAACTCTTAATCAAAGTGTTATACAGCAAACAGTTGAAAT
TTCAGTTATGGTTGAGCAACTTAAAAAGATTATTCAAGAGGTTTTAGGACTTGTTATTAACAGT
ACGTGTAGTAAGAAGTATCCGCGTTCTCCGTGTATGACTAGTTTTTGGAATTCGGTAGAAGCTA
CAATTAAAGGCACATTTACAAATTTAGACACTCAAATAGATGAAGCATGGATTTTTTGGCATAG
TTTATCCGCCCACAATACAAGTTATTATTATAATATTTTATTTTCTATTCAAAATGAAGATACA
GGTGCAGTTATGGCAGTATTACCTTTAGCATTTGAGGTTTCTGTGGATGTTGAAAAACAAAAAG
TATTATTCTTTACAATAAAAGATAGTGCACGATATGAAGTTAAAATGAAAGCTTTGACTTTAGT
TCAAGCTCTACATTCCTCTGATGCCCCAATTGTAGATATATTTAATGTTAATAACTATAATTTA
TACCATTCTAATCATAAGATTATTCAAAATTTAAATTTATCGAATTGA

Amino acid sequence of CGAL3 (SEQ ID NO:32)

MYTKNFSNSRMEVKGNNGCSAPIIRKPFKHIVLTVPSSDLDNFNTVFYVQPQYINQALHLANAF
QGAIDPLNLNFNFEKALQIANGIPNSAIVKTLNQSVIQQTVEISVMVEQLKKIIQEVLGLVINS
TCSKKYPRSPCMTSFWNSVEATIKGTFTNLDTQIDEAWIFWHSLSAHNTSYYYNILFSIQNEDT
GAVMAVLPLAFEVSVDVEKQKVLFFTIKDSARYEVKMKALTLVQALHSSDAPIVDIFNVNNYNL
YHSNHKIIQNLNLSN

Nucleotide Sequence encoding CGAL4 (SEQ ID NO:33)

ATGTATACTAAAAATTTTAGTAATTCCAGAATGGAAGTAAAAGGTAATAACGGCTGTTCTGCAC
CTATTATTAGAAAACCATTTAAACATATTGTATTAACGGTTCCATCCAGTGATTTAGATAATTT
TAATACAGTCTTTTATGTACAACCACAATACATTAATCAGGCTCTTCATTTAGCAAATGCTTTT
CAAGGGGCTATAGACCCACTTAATTTAAATTTCAATTTTGAAAAGGCACTCCAAATTGCAAATG
GTATTCCTAATTCTGCAATTGTAAAAACTCTTAATCAAAGTGTTATACAGCAAACAGTTGAAAT
TTCAGTTATGGTTGAGCAACTTAAAAAGATTATTCAAGAGGTTTTAGGACTTGTTATTAACAGT
ACTAGTTTTTGGAATTCGGTAGAAGCTACAATTAAAGGCACATTTACAAATTTAGACACTCAAA
TAGATGAAGCATGGATTTTTTGGCATAGTTTATCCGCCCACAATACGTGTAGTAAGAAGTATCC
GCGTTCTCCGTGTATGACAAGTTATTATTATAATATTTTATTTTCTATTCAAAATGAAGATACA
GGTGCAGTTATGGCAGTATTACCTTTAGCATTTGAGGTTTCTGTGGATGTTGAAAAACAAAAAG
TATTATTCTTTACAATAAAAGATAGTGCACGATATGAAGTTAAAATGAAAGCTTTGACTTTAGT
TCAAGCTCTACATTCCTCTGATGCCCCAATTGTAGATATATTTAATGTTAATAACTATAATTTA
TACCATTCTAATCATAAGATTATTCAAAATTTAAATTTATCGAATTGA

Amino acid sequence of CGAL4 (SEQ ID NO:34)

MYTKNFSNSRMEVKGNNGCSAPIIRKPFKHIVLTVPSSDLDNFNTVF
YVQPQYINQALHLANAFQGAIDPLNLNFNFEKALQIANGIPNSAIVK
TLNQSVIQQTVEISVMVEQLKKIIQEVLGLVINSTSFWNSVEATIKG
TFTNLDTQIDEAWIFWHSLSAHNTCSKKYPRSPCMTSYYYNILFSIQ
NEDTGAVMAVLPLAFEVSVDVEKQKVLFFTIKDSARYEVKMKALTLV
QALHSSDAPIVDIFNVNNYNLYHSNHKIIQNLNLSN

Amino acid sequence of CGSL1 (SEQ ID NO:35)

MYTKNFSNSRMEVKGNNGCSAPIIRKPFKHIVLTVPTCSKKYPRSPCM
NTVFYVQPQYINQALHLANAFQGAIDPLNLNFNFEKALQIANGIPNSA
IVKTLNQSVIQQTVEISVMVEQLKKIIQEVLGLVINSTSFWNSVEATI
KGTFTNLDTQIDEAWIFWHSLSAHNTSYYYNILFSIQNEDTGAVMAVL
PLAFEVSVDVEKQKVLFFTIKDSARYEVKMKALTLVQALHSSDAPIVD
IFNVNNYNLYHSNHKIIQNLNLSN

Amino acid sequence of CGSL2 (SEQ ID NO:36)

MYTKNFSNSRMEVKGNNGCSAPIIRKPFKHIVLTVPSSDLDNFNTVF
YVQPQYINQALHLANTCSKKYPRSPCMFEKALQIANGIPNSAIVKTL
NQSVIQQTVEISVMVEQLKKIIQEVLGLVINSTSFWNSVEATIKGTF
TNLDTQIDEAWIFWHSLSAHNTSYYYNILFSIQNEDTGAVMAVLPLA
FEVSVDVEKQKVLFFTIKDSARYEVKMKALTLVQALHSSDAPIVDIF
NVNNYNLYHSNHKIIQNLNLSN

Amino acid sequence of CGSL3 (SEQ ID NO:37)

MYTKNFSNSRMEVKGNNGCSAPIIRKPFKHIVLTVPSSDLDNFNTVF
YVQPQYINQALHLANAFQGAIDPLNLNFNFEKALQIANGIPNSAIVK
TLNQSVIQQTVEISVMVEQLKKIIQEVTCSKKYPRSPCMTSFWNSVE
ATIKGTFTNLDTQIDEAWIFWHSLSAHNTSYYYNILFSIQNEDTGAV
MAVLPLAFEVSVDVEKQKVLFFTIKDSARYEVKMKALTLVQALHSSD
APIVDIFNVNNYNLYHSNHKIIQNLNLSN

Amino acid sequence of CGSL4 (SEQ ID NO:38)

MYTKNFSNSRMEVKGNNGCSAPIIRKPFKHIVLTVPSSDLDNFNTVFY
VQPQYINQALHLANAFQGAIDPLNLNFNFEKALQIANGIPNSAIVKTL
NQSVIQQTVEISVMVEQLKKIIQEVLGLVINSTSFWNSVEATIKGTFT
NLDTQIDEAWIFWH TCSKKYPRSPCM TSYYYNILFSIQNEDTGAVM
AVLPLAFEVSVDVEKQKVLFFTIKDSARYEVKMKALTLVQALHSSDAP
IVDIFNVNNYNLYHSNHKIIQNLNLSN

Amino acid sequence of CGSL5): (SEQ ID NO:39)

MYTKNFSNSRMEVKGNNGCSAPIIRKPFKHIVLTVPSSDLDNFNTVF
YVQPQYINQALHLANAFQGAIDPLNLNFNFEKALQIANGIPNSAIVK
TLNQSVIQQTVEISVMVEQLKKIIQEVLGLVINSTSFWNSVEATIKG
TFTNLDTQIDEAWIFWHSLSAHNTSYYYNILFSIQTCSKKYPRSPCM
AVMAVLPLAFEVSVDVEKQKVLFFTIKDSARYEVKMKALTLVQALHS
SDAPIVDIFNVNNYNLYHSNHKIIQNLNLSN

Amino acid sequence of CGSL7: (SEQ ID NO:40)

MYTKNFSNSRMEVKGNNGCSAPIIRKPFKHIVLTVPSSDLDNFNTVF
YVQPQYINQALHLANAFQGAIDPLNLNFNFEKALQIANGIPNSAIVK
TLNQSVIQQTVEISVMVEQLKKIIQEVLGLVINSTSFWNSVEATIKG
TFTNLDTQIDEAWIFWHSLSAHNTSYYYNILFSIQNEDTGAVMAVLP
LAFEVSVDVEKQKVLFFTIKDSARYEVKMKALTLVQAL TCSKKYPR
SPCM IVDIFNVNNYNLYHSNHKIIQNLNLSN

It is understood that a different gut binding peptide fitting the consensus sequence of SEQ ID NO:21, or any of SEQ ID NOs:2-20 can be inserted in place of TCSKKYPRSPCM (SEQ ID NO:11) into the same sites in the above exemplified proteins above, with the result that chimeric insecticidal toxins are given. The particular gut binding peptide can be tailored to a target insect of choice.

Amino acid sequence (ACF35049) (SEQ ID NO:28):

MYTKNFSNSRMEVKGNNGCSAPIIRKPFKHIVLTVPSSDLDNFNTVFYVQPQYINQALHLANAFQGAID
PLNLNFNFEKALQIANGIPNSAIVKTLNQSVIQQTVEISVMVEQLKKIIQEVLGLVINSTSFWNSVEAT
IKGTFTNLDTQIDEAWIFWHSLSAHNTSYYYNILFSIQNEDTGAVMAVLPLAFEVSVDVEKQKVLFFTI
KDSARYEVKMKALTLVQALHSSDAPIVDIFNVNNYNLYHSNHKIIQNLNLSN
Loop 1: amino acids 37 to 43 of SEQ ID NO: 28 (SSDLDNF)
Loop 2: amino acids 63 to 76 of SEQ ID NO: 28 (AFQGAIDPLNLNFN)
Loop 3: amino acids 122 to 128 of SEQ ID NO: 28 (LGLVINS)
Loop 4: amino acids 159 to 164 of SEQ ID NO: 28 (SLSAHN)
Loop 5: amino acids 177 to 181 of SEQ ID NO: 28 (NEDTG)
Loop 7: amino acids 227 to 232 of SEQ ID NO: 28 (HSSDAP)

TABLE 5
Cyt2Aa substitution variant chimeric toxins and amino
acids replaced for respective chimeric toxins

| | amino acids of Cyt2Aa |
|---|---|
| Cyt2Aa-GBP3.1 | (SEQ ID NO: 28) replaced by |
| chimeric toxin | GBP3.1 in CGSLn mutants |
| CGSL1 | ³⁷SSDLDNF⁴³ |
| CGSL2 | ⁶³AFQGAIDPLNLNFN⁷⁶ |
| CGSL3 | ¹²²LGLVINS¹²⁸ |
| CGSL4 | ¹⁵⁹SLSAHN¹⁶⁴ |
| CGSL5 | ¹⁷⁷NEDTG¹⁸¹ |
| CGSL7 | ²²⁷HSSDAP²³² |

Cry4A amino acid sequence as specifically exemplified (SEQ ID NO:41) with sites for insertion of a gut binding peptide marked by arrows. A peptide of SEQ ID NO:1-20 or one fitting consensus sequence SEQ ID NO:21 or other target insect gut binding peptide can be inserted at one or more of the marked positions. Arrows indicate the sites where at least one gut binding peptide is added.

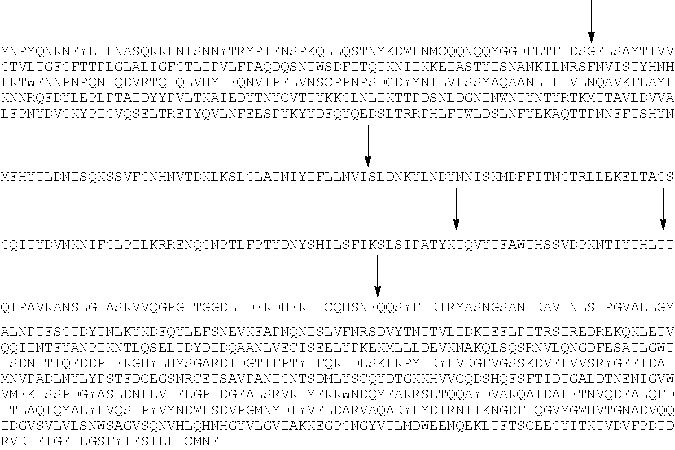

Cry4Aa: peptide substitution sites in SEQ ID NO:41. Each box containing amino acids is replaced by a gut binding peptide, for example, one or more of SEQ ID NO:1-21 for chimeric aphicidal toxin or other gut binding peptide to form a chimeric insecticidal toxin active against a target insect of interest.

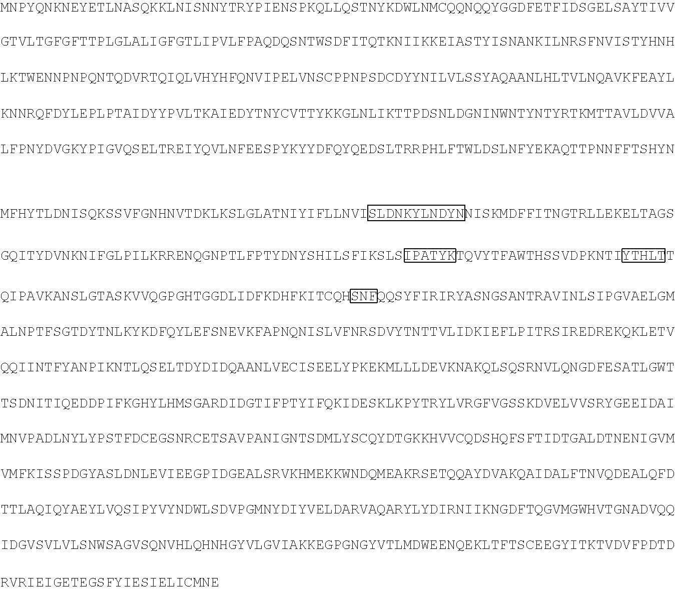

Cry4Aa Coding Sequence (Y00423.1) (SEQ ID NO:42)

TGAATCCTTATCAAAATAAAAATGAATATGAAACATTAAATGCTTCACAAAAAAAATTAAATAT
ATCTAATAATTATACAAGATATCCAATAGAAAATAGTCCAAAACAATTATTACAAAGTACAAAT
TATAAAGATTGGCTCAATATGTGTCAACAGAATCAGCAGTATGGTGGAGATTTTGAAACTTTTA
TTGATAGTGGTGAACTCAGTGCCTATACTATTGTAGTTGGGACCGTACTGACTGGTTTCGGGTT
CACAACACCCTTAGGACTTGCTTTAATAGGTTTTGGTACATTAATACCAGTTCTTTTTCCAGCC
CAAGACCAATCTAACACATGGAGTGACTTTATAACACAAACTAAAAATATTATAAAAAAAGAAA
TAGCATCAACATATATAAGTAATGCTAATAAAATTTTAAACAGGTCGTTTAATGTTATCAGCAC
TTATCATAATCACCTTAAAACATGGGAGAATAATCCAAACCCACAAAATACTCAGGATGTAAGG
ACACAAATCCAGCTAGTTCATTACCATTTTCAAAATGTCATTCCAGAGCTTGTAAACTCTTGTC
CTCCTAATCCTAGTGATTGCGATTACTATAACATACTAGTATTATCTAGTTATGCACAAGCAGC
AAACTTACATCTGACTGTATTAAATCAAGCCGTCAAATTTGAAGCGTATTTAAAAAACAATCGA
CAATTCGATTATTTAGAGCCTTTGCCAACAGCAATTGATTATTATCCAGTATTGACTAAAGCTA
TAGAAGATTACACTAATTATTGTGTAACAACTTATAAAAAAGGATTAAATTTAATTAAAACGAC
GCCTGATAGTAATCTTGATGGAAATATAAACTGGAACACATACAATACGTATCGAACAAAAATG
ACTACTGCTGTATTAGATGTTGTTGCACTCTTTCCTAATTATGATGTAGGTAAATATCCAATAG
GTGTCCAATCTGAACTTACTCGAGAAATTTATCAGGTACTTAACTTCGAAGAAAGCCCCTATAA
ATATTATGACTTTCAATATCAAGAGGATTCACTTACACGTAGACCGCATTTATTTACTTGGCTT
GATTCTTTGAATTTTTATGAAAAAGCGCAAACTACTCCTAATAATTTTTTCACCAGCCATTATA
ATATGTTTCATTACACACTTGATAATATATCCCAAAAATCTAGTGTTTTTGGAAATCACAATGT
AACTGATAAATTAAAATCTCTTGGTTTGGCAACAAATATTTATATTTTTTTATTAAATGTCATA
AGCTTAGATAATAAATATCTAAATGATTATAATAATATTAGTAAAATGGATTTTTTTATAACTA
ATGGTACTAGACTTTTGGAGAAAGAACTTACAGCAGGATCTGGGCAAATAACTTATGATGTAAA
TAAAAATATTTTCGGGTTACCAATTCTTAAACGAAGAGAGAATCAAGGAAACCCTACCCTTTTT
CCAACATATGATAACTATAGTCATATTTTATCATTTATTAAAAGTCTTAGTATCCCTGCAACAT
ATAAAACTCAAGTGTATACGTTTGCTTGGACACACTCTAGTGTTGATCCTAAAAATACAATTTA
TACACATTTAACTACCCAAATTCCAGCTGTAAAAGCGAATTCACTTGGGACTGCTTCTAAGGTT
GTTCAAGGACCTGGTCATACAGGAGGGGATTTAATTGATTTCAAAGATCATTTCAAAATTACAT
GTCAACACTCAAATTTTCAACAATCGTATTTTATAAGAATTCGTTATGCTTCAAATGGAAGCGC
AAATACTCGAGCTGTTATAAATCTTAGTATCCCAGGGGTAGCAGAACTGGGTATGGCACTCAAC
CCCACTTTTTCTGGTACAGATTATACGAATTTAAAATATAAAGATTTTCAGTACTTAGAATTTT
CTAACGAGGTGAAATTTGCTCCAAATCAAAACATATCTCTTGTGTTTAATCGTTCGGATGTATA
TACAAACACAACAGTACTTATTGATAAAATTGAATTTCTGCCAATTACTCGTTCTATAAGAGAG
GATAGAGAGAAACAAAAATTAGAAACAGTACAACAAATAATTAATACATTTTATGCAAATCCTA
TAAAAAACACTTTACAATCAGAACTTACAGATTATGACATAGATCAAGCCGCAAATCTTGTGGA
ATGTATTTCTGAAGAATTATATCCAAAAGAAAAAATGCTGTTATTAGATGAAGTTAAAAATGCG
AAACAACTTAGTCAATCTCGAAATGTACTTCAAAACGGGGATTTTGAATCGGCTACGCTTGGTT
GGACAACAAGTGATAATATCACAATTCAAGAAGATGATCCTATTTTTAAAGGGCATTACCTTCA
TATGTCTGGGGCGAGAGACATTGATGGTACGATATTTCCGACCTATATATTCCAAAAAATTGAT
GAATCAAAATTAAAACCGTATACACGTTACCTAGTAAGGGGATTTGTAGGAAGTAGTAAAGATG
TAGAACTAGTGGTTTCACGCTATGGGGAAGAAATTGATGCCATCATGAATGTTCCAGCTGATTT
AAACTATCTGTATCCTTCTACCTTTGATTGTGAAGGGTCTAATCGTTGTGAGACGTCCGCTGTG
CCGGCTAACATTGGGAACACTTCTGATATGTTGTATTCATGCCAATATGATACAGGGAAAAAGC
ATGTCGTATGTCAGGATTCCCATCAATTTAGTTTCACTATTGATACAGGGGCATTAGATACAAA
TGAAAATATAGGGGTTTGGGTCATGTTTAAAATATCTTCTCCAGATGGATACGCATCATTAGAT
AATTTAGAAGTAATTGAAGAAGGGCCAATAGATGGGGAAGCACTGTCACGCGTGAAACACATGG
AGAAGAAATGGAACGATCAAATGGAAGCAAAACGTTCGGAAACACAACAAGCATATGATGTAGC
GAAACAAGCCATTGATGCTTTATTCACAAATGTACAAGATGAGGCTTTACAGTTTGATACGACA
CTCGCTCAAATTCAGTACGCTGAGTATTTGGTACAATCGATTCCATATGTGTACAATGATTGGT
TGTCAGATGTTCCAGGTATGAATTATGATATCTATGTAGAGTTGGATGCACGAGTGGCACAAGC
GCGTTATTTGTATGATATAAGAAATATTATTAAAAATGGTGATTTTACACAAGGGGTAATGGGG
TGGCATGTAACTGGAAATGCAGACGTACAACAAATAGATGGTGTTTCTGTATTGGTTCTATCTA
ATTGGAGTGCTGGCGTATCTCAAAATGTCCATCTCCAACATAATCATGGGTATGTCTTAGGTGT
TATTGCCAAAAAAGAAGGACCTGGAAATGGGTATGTCACGCTTATGGATTGGGAGGAGAATCAA
GAAAAATTGACGTTTACGTCTTGTGAAGAAGGATATATTACGAAGACAGTAGATGTATTCCCAG
ATACAGATCGTGTACGAATTGAGATAGGCGAAACCGAAGGTTCGTTTTATATCGAAAGCATTGA
ATTAATTTGCATGAACGAGTGA

Another Cry4A insecticidal protein has a sequence as set forth in NCBI sequence EF424469.1, EF424468.1, EF208904.1 or HC732056.1 or an amino acid sequence with at least 85% amino acid sequence identity to one of the foregoing.

Amino acid sequence of Cry4A from ABM97547.1 (SEQ ID NO:43)

| | |
|---|---|
| 1 | mnpyqnkney etlnasqkkl nisnnytryp ienspkqllq stnykdwlnm cqqnqqyggd |
| 61 | fetfidsgel saytivvgtv ltgfgfttpl glaligfgtl ipvlfpaqdq sntwsdfitq |
| 121 | tkniikkeia styisnanki lnrsfnvist yhnhlktwen npnpqntqdv rtqiqlvhyh |
| 181 | fqnvipelvn scppnpsdcd yynilvlssy ahaanlhltv lnqavkfeay lknnrqfdyl |
| 241 | eplptaidyy pvltkaiedy tnycvttykk glnlikttpd snldgninwn tyntyrtkmt |
| 301 | tavldvvalf piydvgkypi gvqseltrei yqvinfeesp ykyydfqyqe dsltrrphlf |
| 361 | twldslnfye kaqttpnnff tshynmfhyt ldnisqkssv fgnhnvtdkl kalglatniy |
| 421 | ifllnvisld nkylndynni skmdffitng trllekelta gsgqitydvn knifglpilk |
| 481 | rrenqgnptl fptydnyshi lsfikslsip atyktqvytf awthssvdpk ntiythlttq |
| 541 | ipavkanslg taskvvqgpg htggdlidfk dhfkitcqhs nfqqsyfiri rfasngsant |
| 601 | ravinlsipg vaelgmalnp tfsgtdytnl kykdfqylef snevkfapnq nislvfnrsd |
| 661 | vytnttvlid kieflpitrs iredrekqkl etvqqiintf yanpikntlq seltdydidq |
| 721 | aanlvecise elypkekmll ldevknakql sksrnvlqng dfesatlgwt tsdnitiqed |
| 781 | dpifkghylh msgardidgt ifptyifqki desklkpytr ylvrgfvgss kdvelvvsry |
| 841 | geeidaimhv padlnylyps tcdceasnrc etsavpanig ntsdmlyscq ydtgkkhvvc |
| 901 | qdshqfsfti dtgaldtnen igvwvmfkis spdgyasldn levieegpid gealsrvkhm |
| 961 | ekkwndqmea krsetqqayd vakqaidalf tnvqdealqf dttlaqiqya eylvqsipyv |
| 1021 | yndwlsdvpg mnydiyveld arvaqaryly dirniikngd ftqgvmgwhv tgnadvqqid |
| 1081 | gvsvlvlfnw sagvsqnvhl hhnhgyvlgv iakkegpgng yvtlmdween qekltftsce |
| 1141 | egyitktvdv fpdtdrvrie igetegsfyi esidlicmne |

Amino acid sequence of Cry4A from ABR12214.1 (SEQ ID NO:44)

| | |
|---|---|
| 1 | mnpyqnkney etlnasqkkl nisnnytryp ienspkqllq stnykdwlnm cqqnqqyggd |
| 61 | fetfidsgel saytivvgtv ltgfgfttpl glaligfgtl ipvlfpaqdq sntwsdfitq |
| 121 | tkniikkeia styisnanki lnrsfnvist yhnhlktwen npnpqntqdv rtgiqlvhyh |
| 181 | fqnvipelvn scppnpsdcd yynilvlssy aqaanlhltv lnqavkfeay lknnrqfdyl |
| 241 | eplptaidyy pvltkaiedy tnycvttykk gfnlikttpd snldgninwi tyntyrtkmt |
| 301 | tavldvvalf piydvgkypi gvqseltrei yqvinfeesp ykyydfqyqe dsltrrphlf |
| 361 | twldslnfye kaqttpnnff tshynmfhyt ldnisqkssv fgnhnvtdkl kalglatniy |
| 421 | ifllnvisld nkylndynni skmdffitng trllekelta gsgqitydvi knifglpilk |
| 481 | rrenqgnptf fptydnyshi lsfikslsip atyktqvytf awthssvdpk ntiythlttq |
| 541 | ipavkanslg taskvvqgpg htggdlidfk vhfkitcqhs nfqqsyfiri rfasngsant |
| 601 | ravinlsipg vaelgmalnp tfsgtdytnl kykdfqylef snevkfapnq nislvfnrsd |
| 661 | vytnttvlid kieflpitrs iredrekqkl etvqqiintf yanpikntlq seltdydidq |
| 721 | aanlvecise elypkekmll ldevknakql sksrnvlqng dfesatlgwt tsdnitiqed |
| 781 | dpifkghylh msgardidgt ifptyifqki desklkpytr ylvrgfvgss kdvelvvsry |
| 841 | geeidgimhv padlnylyps tcdceasnrc etsavpanig ntsdmlyscq ydtgkkhvvc |
| 901 | gdshgfifti dtgaldtnen igvwvmfkis spdgyasldn levieegpid gealsrvkhm |
| 961 | ekkwndqmea krsetqqayd vakqaidalf tnvqdealqf dttlaqiqya eylvqsipyv |
| 1021 | yndwlsdvpg mnydiyveld arvagaryly dirniikngd ftqgvmgwdv tgnadvggid |
| 1081 | gvsvlvlfnw sagvsqnvhl hhnhgyvlgv iakkegpgng yvtlmdween qekltftsce |
| 1141 | egyitktvdv fpdtdrvrie igetegsfyi esidlicmne |

Amino acid sequence of Cry4A from ABR12215.1 (SEQ ID NO:45)

| | |
|---|---|
| 1 | mnpyqnkney etlnasqkkl nisnnytryp ienspkqllq stnykdwlnm cqqnqqyggd |
| 61 | fetfidsgel saytivvgtv ltgfgfttpl glaligfgtl ipvlfpaqdq sntwsdfitq |
| 121 | tkniikkeia styisnanki lnrsfnvist yhnhlktwen npnpqntqdv rtgiqlvhyh |
| 181 | fqnvipelvn scppnpsdcd yynilvlssy ahaanlhltv lnqavkfeay lknnrqfdyl |
| 241 | eplptaidyy pvltkaiedy tnycvttykk glnlikttpd snldgninwn tyntyrtkmt |
| 301 | tavldvvalf piydvgkypi gvqseltrei yqvinfeesp ykyydfqyqe dsltrrphlf |
| 361 | twldslnfye kaqttpnnff tshynmfhyt fdnisqkssv fgnhnvtdkl kalglatniy |
| 421 | ifllnvisld nkylkdynni skmdffitng trlwekelta gsgqitydvn knifglpilk |
| 481 | rrenqgnptl fatydnyshi lsfikslsir atyktqvytf awthssvdpk ntiythlttq |
| 541 | ipavkanslg taskgvqgpg htggdlidfk dhfkitcqhs nfqqsyfiri rfasngsant |
| 601 | ravinlsipg vaelgmalnp tfsgtdytnl kykdfqylef snevkfapnq nislvfnrsd |
| 661 | vytnttvlid kieflpitrs iredrekqkl etvqqiintf yanpikntlq seltdydidq |
| 721 | aanlvecise elypkekmll ldevknakql sksrnvlqng dfesatlgwt ksdnitiqed |
| 781 | dpifkghylh rsgardidgt ifptyifqki desklkpytr ylvrgfvgss kdvelvvsry |
| 841 | geeidaimhf padlnylyps tcdceasnrc etsavpanig ntsdmlyscq ydtgkkhvvc |
| 901 | qdshqfsfti dtgaldtnen igvwvmfkis spdgyasldn levieegpid gealsrvkhm |
| 961 | ekkwndqmea krsetqqayd vakqaiealf tnvqdealqf dttlaqiqya eylvqsipyv |
| 1021 | yndwlsdvpg mnydiyveld arvagaryly dirniikngd ftqgvmgwhv tgnadvggid |
| 1081 | gvsvlvlfnw ragvsqnvhl hhnhgyvlgv iakkegpgng yvtlmdween qekltftsce |
| 1141 | egyitktvdv fpdtdrvrie igetegsfyi esidlicmne |

Amino acid sequence of Cry4A from ABR12217.1 (SEQ ID NO:46)

| | |
|---|---|
| 1 | mnpycinkney etlnasqkkl nisnnytrypienspkqllq stnykdwlnm cqqnqqyggd |
| 61 | fetfidsgel saytivvgtv ltgfgfttpl glaligfgtl ipvlfpaqdq sntwsdfitq |
| 121 | tkniikkeia styisnanki lnrsfnvist yhnhlktwen npnpqntqdv rtgiqlvhyh |
| 181 | fqnvipelvn scppnpsdcd yynilvlssy ahaanlhltv lnqavkfeay lknnrqfdyl |
| 241 | eplptaidyy pvltkaiedy tnycvttykk glnlikttpd snldgninwn tyntyrtkmt |
| 301 | tavldvvalf piydvgkypi gvqseltrei yqvinfeesp ykyydfqyqe dsltrrphlf |
| 361 | twldslnfye kaqttpnnff tshynmfhyt fdnisqkssv fgnhnvtdkl kalglatniy |
| 421 | ifllnvisld nkylkdynni skmdffitng trlwekelta gsgqitydvn knifglpilk |
| 481 | rrenqgnptl fatydnyshi lsfikslsir atyktqvytf awthssvdpk ntiythlttq |
| 541 | ipavkanslg taskgvqgpg htggdlidfk dhfkitcqhs nfqqsyfiri rfasngsant |
| 601 | ravinlsipg vaelgmalnp tfsgtdytnl kykdfqylef snevkfapnq nislvfnrsd |
| 661 | vytnttvlid kieflpitrs iredrekqkl etvqqiintf yanpikntlq seltdydidq |
| 721 | aanlvecise elypkekmll ldevknakql sksrnvlqng dfesatlgwt ksdnitiqed |
| 781 | dpifkghylh rsgardidgt ifptyifqki desklkpytr ylvrgfvgss kdvelvvsry |
| 841 | geeidaimhf padlnylyps tcdceasnrc etsavpanig ntsdmlyscq ydtgkkhvvc |
| 901 | qdshqfsfti dtgaldtnen igvwvmfkis spdgyasldn levieegpid gealsrvkhm |
| 961 | ekkwndqmea krsetqqayd vakqaiealf tnvqdealqf dttlaqiqya eylvqsipyv |
| 1021 | yndwlsdvpg mnydiyveld arvagaryly dirniikngd ftqgvmgwhv tgnadvggid |
| 1081 | gvsvlvlfnw ragvsqnvhl hhnhgyvlgv iakkegpgng yvtlmdween qekltftsce |
| 1141 | egyitktvdv fpdtdrvrie igetegsfyi esidlicmne |

Amino acid sequence of Cry4A from ABR12218.1 (SEQ ID NO:47)

| | |
|---|---|
| 1 | mnpyqnkney etlnasqkkl nisnnytryp ienspkqllq stnykdwlnm cqqnqqyggd |
| 61 | fetfidsgel saytivvgtv ltgfgfttpl glaligfgtl ipvlfpagdp sntwsdfitq |
| 121 | tkniikkeia styisnanki lnrsfnvist yhnhlktwen npnpqntqgv rtgiqlvhyh |
| 181 | fqnvipelvn scppnpsdcd yynilvlssy ahaanlhltv lnqavnfeay lknnrqfdyl |
| 241 | eplptaidyy pvltkaiedy tnycvttykk glnlikttpd snldgninwn tyntyrtkmt |
| 301 | tavldvvalf piydvgkypi gvqseltrei yqvinfeesp ykyydfqyqe dsltrrphlf |
| 361 | twldslnfye kaqttpnnff tshynmfhyt ldnisqkssv fgnhnetdkl kalglatniy |
| 421 | ifllnvisld nkylndynni skmdffitng trllekelta gsgqitydvn knifglpilk |
| 481 | rrenqgnptl fptydnyshi lsfikslsip atyktqvytf awthssvdpk ntiythlttq |
| 541 | ipavkanslg taskvvqgpg htggdlidfk dhfkitcqhs nfqqsyfiri rfasngsant |
| 601 | ravinlsipg vaelgmalnp tfsgtdytkl kykdfqylef snevkfapnq nislvfnrsd |
| 661 | vytnttvlid kieflpitrs iredrekqkl etvqqiintf yanpikntlq seltdydidq |
| 721 | aanlvecise elypkekmll ldevknakql sksrnvlqng dfepatlgwt tsdnitiqed |
| 781 | dpifkghylh msgardidgt ifptyifqki desklkpytr ylvrgfvgss kdvelvvsry |
| 841 | geeidaimhv padlnylyps tcdceasnrc etsavpanig ntsdmlyscq ydtgkkhvvc |
| 901 | qdshqfsfti dtgaldtnen igvwvmfkis spdgyasldn levieegpid gealsrvkhm |
| 961 | ekkwndqmea krsetqqayd vakqaidalf tnvqdealqf dttlaqiqya eylvqsipyv |
| 1021 | yndwlsdvpg mnydiyveld arvaqaryly dirniikngd ftqgvmgwhv tgnadvqqid |
| 1081 | gvsvlvlfnw sagvsqnvhl hqnhgyvlgv iakkegpgng yvtlmdween qekltftsce |
| 1141 | egyitktvdv fpdtdrvrie igetevsfyi esidlicmne |

Amino acid sequence of Cry4A from ABR12216.1 (SEQ ID NO:48)

| | |
|---|---|
| 1 | mnpyqnkney etlnasqkkl nisnnytryp ienspkqllq stnykdwlnm cqqnqqyggd |
| 61 | fetfidsgel caytivvgtv ltgfgfttpl glaligfgtl ipvlfpaqdq sntwsdfitq |
| 121 | tkniikkeia styisnanki lnrsfnvist yhnhlktwen npnpqntqdv rtgiqlvhyh |
| 181 | fqnvipelvn scppnpsdcd yynilvlssy ahaanlhltv lnqavkfeay lknnrqfdyl |
| 241 | eplptaidyy pvltkaiedy tnycvttykk glnlikttpd snldgninwn tyntyrtkmt |
| 301 | tavldvvalf piydvgkypi gvqseltrei yqvinfeesp ykyydfqyqe dsltrrphlf |
| 361 | twldslnfye kaqttpnnff tshynmflyt ldnisqkssv fgnhnvtdkl kalglatniy |
| 421 | ifllnvisld nkylndynni skmdffitng trllekelta gsgqitydvn knifglpilk |
| 481 | rrenqgnptl fptydnyshi lsfikslsip etyktqvytf awthssvdpk ntiythlttq |
| 541 | ipavkanslg taskvvqgpg htggdlidfk dhfkitcqhs nfqqsyfiri rfasngsant |
| 601 | ravinlsipr vaelgmalnp tfsgtdytnl kykdfqylef snevkfapnq nislvfnrsd |
| 661 | vytnttvlid kieflpitrs iredrekqkl etvqqiintf yanpikntlq seltdydidq |
| 721 | aanlvecise elypkekmll ldevknakql sksrnvlqng dfesatlgwt tsdnitiqed |
| 781 | dpifkghylh msgardidgt ifptyifqki desklkpytr ylvrgfvgss kdvelvvsry |
| 841 | geeidaimhv padlnylyps tcdceasnrc etsavpanig ntsdmlyscq ydtgkkhvvc |
| 901 | qdshqfsfti dtgaldtnen igvwvmfkis spdgyasldn levieegpid gealsrvkhm |
| 961 | ekkwndqmea krsetqqayd vakqaidalf tnvqdealqf dttlaqiqya eylvqsipyv |
| 1021 | yndwlsdvpg mnydiyveld arvagaryly dirniikngd ftqgvmgwhv tgnadvggid |
| 1081 | gvsvlvlfnw sagvsqnvhl hhnhgyvlgv iakkegpgng yvtlmdween qekltftsce |
| 1141 | egyitktvdv fpdtdrvrie igetegsfyi esidlicmne |

Results of Amino Acid Sequence Alignment for Cry4A

Sequence type explicitly set to Protein
Sequence format is Pearson

### Sequence 1 (SEQ ID NO:43): ABM97547.1 1180 aa ISEQ ID NO:43)

### Sequence 2 (SEQ ID NO:44): ABR12214.1 1180 aa

### Sequence 3 (SEQ ID NO:45): ABR12215.1 1180 aa (SEQ ID NO:45)

### Sequence 4(SEQ ID NO:46): ABR12217.1 1180 aa (SEQ ID NO:46)

### Sequence 5 (SEQ ID NO:47): ABR12218.1 1180 aa (SEQ ID NO:47)

### Sequence 6(SEQ ID NO:48): ABR12216.1 1180 aa (SEQ ID NO:49)

### Sequence 7: CAA68485.1 1180 aa

Start of Pairwise alignments
Sequences (1:2) Aligned. Score: 99
Sequences (1:3) Aligned. Score: 99
Sequences (1:4) Aligned. Score: 99
Sequences (1:5) Aligned. Score: 99
Sequences (1:6) Aligned. Score: 99
Sequences (1:7) Aligned. Score: 99
Sequences (2:3) Aligned. Score: 98
Sequences (2:4) Aligned. Score: 98
Sequences (2:5) Aligned. Score: 98
Sequences (2:6) Aligned. Score: 98
Sequences (2:7) Aligned. Score: 98
Sequences (3:4) Aligned. Score: 100
Sequences (3:5) Aligned. Score: 98
Sequences (3:6) Aligned. Score: 98
Sequences (3:7) Aligned. Score: 98
Sequences (4:5) Aligned. Score: 98
Sequences (4:6) Aligned. Score: 98
Sequences (4:7) Aligned. Score: 98
Sequences (5:6) Aligned. Score: 98
Sequences (5:7) Aligned. Score: 98
Sequences (6:7) Aligned. Score: 98
Guide tree file created: [clustalw2-120110607-213724-0386-79407485-pg.dnd]

There are 6 groups

### Start of Multiple Alignment

### Group 1: Sequences: 2 Score:25509

### Group 2: Sequences: 3 Score:25558

### Group 3: Sequences: 4 Score:25631

### Group 4: Sequences: 5 Score:25561

### Group 5: Sequences: 2 Score:25738

### Group 6: Sequences: 7 Score:25522

### Alignment Score 151262

CLUSTAL-Alignment file created [clustalw2-120110607-213724-0386-79407485-pg.aln]

### Bibliography

Ali M I, Luttrell R G, Young S Y, 3rd. 2006. Susceptibilities of Helicoverpa zea and Heliothis virescens (Lepidoptera: Noctuidae) populations to Cry1Ac insecticidal protein. J. Econ. Entomol. 99: 164-75
Ashouri, A. 2004a. Seasonal occurrence and relative abundance of aphids on potato plants with classical and transgenic characters of resistance to Colorado potato beetle Leptinotarsa decemlineata (Say). Commun Agric Appl Biol Sci, 69, 273-280.
Ashouri, A. 2004b. Transgenic-Bt potato plant resistance to the Colorado potato beetle affect the aphid parasitoid Aphidius nigripes. Commun Agric Appl Biol Sci, 69, 185-189.
Ashouri, A., Michaud, D. & Cloutier, C. 2001. Unexpected effects of different potato resistance factors to the Colorado potato beetle (Coleoptera: Chrysolmelidae) on the potato aphid (Homoptera: Aphididae). Environ. Entomol., 30, 524-532.
Auclair, J. L. 1989. Host plant resistance. In Aphids. Their biology, natural enemies and control, Vol. C (Eds. Minks, A. K. & Harrewijn, P.), pp. 225-265. Elsevier, Amsterdam.
Bayyareddy K, Andacht T M, Abdullah M A, Adang M J. 2009. Proteomic identification of Bacillus thuringiensis subsp. israelensis toxin Cry4Ba binding proteins in midgut membranes from Aedes (Stegomyia) aegypti Linnaeus (Diptera, Culicidae) larvae. Insect Biochemistry and Molecular Biology 39: 279-86
Blackman, R. L. 2000a. Aphids on the world's crops. An identification and information guide. John Wiley & Sons, New York.
Blackman, R. L. 2000b. Aphids on the world's crops. An identification and information guide. John Wiley and Sons, New York.
Boonserm P, Mo M, Angsuthanasombat C, Lescar J. 2006. Structure of the functional form of the mosquito larvicidal Cry4Aa toxin from Bacillus thuringiensis at a 2.8-angstrom resolution. J. Bacteriol. 188: 3391-401
Bravo, A., Gomez, I., Conde, J. et al. 2004. Oligomerization triggers binding of a Bacillus thuringiensis Cry1Ab pore-forming toxin to aminopeptidase N receptor leading to insertion into membrane microdomains. Biochim Biophys Acta, 1667, 38-46.
Burgio, G., Lanzoni, A., Accinelli, G. et al. 2007. Evaluation of Bt-toxin uptake by the non-target herbivore, Myzus persicae (Hemiptera: Aphididae), feeding on transgenic oilseed rape. Bull. Entomol. Res., 97, 211-215.
Butko, P. 2003. Cytolytic toxin Cyt1A and its mechanism of membrane damage: Data and hypotheses. Appl Environ Microbiol, 69, 2415-2422.
Cevik, V. & King, G. J. 2002. High-resolution genetic analysis of the Sd-1 aphid resistance locus in Malus spp. Theor. Appl. Genet., 105, 346-354.
Chay C A, Gunasinge U B, Dinesh-Kumar S P, Miller W A, Gray S M. 1996. Aphid transmission and systemic plant infection determinants of barley yellow dwarf luteovirus-PAV are contained in the coat protein readthrough domain and 17-kDa protein, respectively. Virology 219: 57-65.
Czjzek M, Bolam D N, Mosbah A, Allouch J, Fontes C M, Ferreira L M, Bornet O, Zamboni V, Darbon H, Smith N L, Black G W, Henrissat B, Gilbert H J. 2001. The location of the ligand-binding site of carbohydrate-binding modules that have evolved from a common sequence is not conserved. J. Biol. Chem. 276: 48580-7
Christian M, Cermak T, Doyle E L, Schmidt C, Zhang F, Hummel A, Bogdanove A J, Voytas D F. 2010. Targeting DNA double-strand breaks with TAL effector nucleases. Genetics. 2010 October; 186(2):757-61. Epub 2010 Jul. 26.
Christou, P., Capell, T., Kohli, A., Gatehouse, J. A. & Gatehouse, A. M. 2006. Recent developments and future prospects in insect pest control in transgenic crops. Trends Plant Sci, 11, 302-308.
Cristofoletti, P. T., De Sousa, F. A., Rahbe, Y. & Terra, W. R. 2006. Characterization of a membrane-bound aminopeptidase purified from Acyrthosiphon pisum midgut cells. A major binding site for toxic mannose lectins. Febs J, 273, 5574-5588.
Devonshire, A. L. 1989. Resistance of aphids to insecticides. In Aphids, their biology, natural enemies and control, Vol. C. (Eds. Minks, A. K. & Harrewijn, P.), pp. 123-139. Elsevier, Amsterdam.
Dinant, S., Ripoll, C., Pieper M., David, C. 2004 Non transactivable phloem specific expression driven by wheat dwarf geminivirus V-sense promoter in transgenic dicotyledonous species. Physiol. Plant. 121 (1):108-116
Duck, N. & Evola, S. 1997. Use of transgenes to increase host plant resistance to insects: Opportunities and challenges. Advances in Insect Control: The role of transgenic plants., 1-20.
Emden, H. V. & Harrington, R. 2007. Aphids as Crop Pests. pp. 717. CABI Publishing, London.
Evans, W. G. and Gyaisco, G. G., (1956). Notes on the biology of the pea aphid. J. Econ. Ent. 49: 878-879
Faria, C. A., Wackers, F. L., Pritchard, J., Barrett, D. A. & Turlings, T. C. 2007. High susceptibility of bt maize to aphids enhances the performance of parasitoids of lepidopteran pests. PLoS One, 2, e600.
Febvay, G., Delobel, B. & Rahbe, Y. 1988. Influence of the Amino-Acid Balance on the Improvement of an Artificial Diet for a Biotype of Acyrthosiphon-Pisum (Homoptera, Aphididae). Can J Zool, 66, 2449-2453.
Foster, S. P., Harrington, R., Dewar, A. M., Denholm, I. & Devonshire, A. L. 2002. Temporal and spatial dynamics of insecticide resistance in Myzus persicae (Hemiptera: Aphididae). Pest Manag Sci, 58, 895-907.
Gatehouse, A. M. R., Down, R. E., Powell, K. S. et al. 1996. Transgenic potato plants with enhanced resistance to the peach-potato aphid Myzus persicae. Entomologia Experimentalis et Applicata, 79, 295-307.
Gatehouse, A. M. R., Powell, K. S., Peumans, W. J., Damme, E. J. M. V. & Gatehouse, J. A. 1995. Insecticidal properties of plant lectins: Their potential in plant protection. In Lectins, Biomedical Perspectives (Eds. Pusztai, A. & Bardocz, S.), pp. 35-57. Taylor and Francis, London, UK.
Gerber D, Shai Y. 2000. Insertion and organization within membranes of the delta-endotoxin pore-forming domain, helix 4-loop-helix 5, and inhibition of its activity by a mutant helix 4 peptide. J. Biol. Chem. 275: 23602-7
Goggin, F. L., Williamson, W. M. & Ullman, D. E. 1998. Variability in the response of Macrosiphum euphorbiae and Myzus persicae (Hemiptera: Aphididae) to the tomato resistance gene Mi. Environ. Entomol., 30, 101-106.
Gomez I., Pardo-Lopez L., Munoz-Garay C., Fernandez L. E., Perez C., Sanchez J., Soberon M., Bravo A. 2007. Role of receptor interaction in the mode of action of insecticidal Cry and Cyt toxins produced by Bacillus thuringiensis. Peptides 28: 169-73.
Graham M. W., Craig S., Waterhouse P. M. 1997. Expression patterns of vascular-specific promoters RoIC and Sh in transgenic potatoes and their use in engineering PLRV-resistant plants. Plant Mol. Biol. 33(4):729-35.
Greene, J. K., Turnipseed, S. G., Sullivan, M. J. & Herzog, G. A. 1999. Boll damage by Southern green stink bug (Hemiptera: Pentatomidae) and tarnished plant bug (Hemiptera: Miridae) caged on transgenic Bacillus thuringiensis cotton. J. Econ. Entomol., 92, 941-944.
Greene, J. K., Turnipseed, S. G., Sullivan, M. J. & May, O. L. 2001. Treatment thresholds for stink bugs (Hemiptera: Pentatomidae) in cotton J. Econ. Entomol., 94, 403-409.
Guo H, Chen X, Zhang H, Fang R, Yuan Z, Zhang Z, Tian Y. 2004. Characterization and activity enhancement of the phloem-specific pumpkin PP2 gene promoter. Transgenic Res. 13(6):559-66.
Hartman, G. L. 2004. Resistance to soybean aphid in soybean germplasm. Crop Science, 44, 98-106.
Hilder, V. A., Powell, K. S., Gatehouse, A. M. R. et al. 1995. Expression of snowdrop lectin in transgenic tobacco plants results in added protection against aphids. Transgenic Res., 4, 18-25.
Hill, C. B., Crull, L., Herman, T. K., Voegtlin, D. J. & Hartman, G. L. 2010. A new soybean aphid (Hemiptera: Aphididae) biotype identified. Journal of economic entomology, 103, 509-515.
Howlader M T, Kagawa Y, Sakai H, Hayakawa T. 2009. Biological properties of loop-replaced mutants of Bacillus thuringiensis mosquitocidal Cry4Aa. Journal of bioscience and bioengineering 108: 179-83
Howlader M T H, Kagawa Y, Miyakawa A, Yamamoto A, Taniguchi T, Hayakawa T, Sakai H.2010. Alanine Scanning Analyses of the Three Major Loops in Domain II of Bacillus thuringiensis Mosquitocidal Toxin Cry4Aa. Appl Environ Microbiol 76: 860-5
Hua G, Zhang R, Abdullah M A, Adang M J. 2008. Anopheles gambiae cadherin AgCad1 binds the Cry4Ba toxin of Bacillus thuringiensis israelensis and a fragment of AgCad1 synergizes toxicity. Biochemistry 47: 5101-5110.
Johnson P E, Joshi M D, Tomme P, Kilburn D G, McIntosh L P. 1996. Structure of the N-terminal cellulose-binding domain of Cellulomonas fimi CenC determined by nuclear magnetic resonance spectroscopy. Biochemistry 35: 14381-94
Jurat-Fuentes, J. L. & Adang, M. J. 2006. Cry toxin mode of action in susceptible and resistant Heliothis virescens larvae. J Invertebr Pathol, 92, 166-171.
Kim, C. H., Schaible, G., Garrett, L., Lubowski, R. & Lee, D. 2008. Economic impacts of the US soybean aphid infestation: a multi-regional competitive dynamic analysis. Agric. Res. Econ. Rev., 37, 227-242.
Klingler, J., Kovalski, I., Silberstein, L., Thompson, G. A. & Perl-Treves, R. 2001. Mapping of cotton-melon aphid resistance in melon. J. Am. Soc. Hort. Sci., 126, 56-63.
Knowles, B. H., Blatt, M. R., Tester, M. et al. 1989. A cytolytic delta-endotoxin from Bacillus thuringiensis var. israelensis forms cation-selective channels in planar lipid bilayers. FEBS letters, 244, 259-262.
Lawo, N. C., Wackers, F. L. & Romeis, J. 2009. Indian Bt cotton varieties do not affect the performance of cotton aphids. PLoS One, 4, e4804.
Leora-Software 1987. POLO-PC, a user's guide to probit and logit analysis. LeOra Software, Berkeley, Calif.
Li, H., Chougule, N. P. & Bonning, B. C. 2011. Interaction of the Bacillus thuringiensis delta endotoxins Cry1Ac and Cry3Aa with the gut of the pea aphid, Acyrthosiphon pisum (Harris). Journal of Invertebrate Pathology, 107, 69-78.
Li, J., Koni, P. A. & Ellar, D. J. 1996. Structure of the mosquitocidal delta-endotoxin CytB from Bacillus thuringiensis sp. kyushuensis and implications for membrane pore formation. J Mol Biol, 257, 129-152.
Liu, S., Sivakumar, S., Sparks, W. 0., Miller, W. A. & Bonning, B. C. 2010. A peptide that binds the pea aphid gut impedes entry of Pea enation mosaic virus into the aphid hemocoel. Virology, 401, 107-116.
Liu, S., B. C. Bonning, et al. (2006). A simple wax-embedding method for isolation of aphid hemolymph for detection of luteoviruses in the hemocoel. J. Virol. Methods 132(1-2): 174-180.
Mehlo, L., Gahakwa, D., Nghia, P. T. et al. 2005. An alternative strategy for sustainable pest resistance in genetically enhanced crops. Proc. Natl. Acad. Sci. USA, 102, 7812-7816.
Mellet, M. A. & Shoeman, A. S. 2007. Effect of Bt cotton on chrysopids, ladybird beetles and their prey: aphids and whiteflies. Indian J. Exp. Biol., 45, 554-562.
Miles, P. W. 1989. Specific responses and damage caused by Aphidoidea. In Aphids. Their biology, natural enemies and control, Vol. C (Eds. Minks, A. K. & Harrewijn, P.), pp. 23-47. Elsevier, Amsterdam.
Park Y, Abdullah M A, Taylor M D, Rahman K, Adang M J. 2009. Enhancement of Bacillus thuringiensis Cry3Aa and Cry3Bb toxicities to coleopteran larvae by a toxin-binding fragment of an insect cadherin. Appl. Environ. Microbiol. 75: 3086-3092
Pascal, T., Pfeiffer, F., Kervella, J., Lacroze, J. P. & Sauge, M. H.2002. Inheritance of green peach aphid resistance in the peach cultivar ‘Rubira’. Plant Breeding, 121, 459-461.
Perez, C., Fernandez, L. E., Sun, J. et al. 2005. Bacillus thuringiensis subsp. israelensis Cyt1Aa synergizes Cry11Aa toxin by functioning as a membrane-bound receptor. Proc Natl Acad Sci USA, 102, 18303-18308.
Poncet S, Delecluse A, Klier A, Rapoport G. 1995. Evaluation of Synergistic Interactions among the Cryiva, Cryivb, and Cryivd Toxic Components of Bacillus-Thuringiensis Subsp Israelensis Crystals. J. Invert. Pathol. 66: 131-135
Porcar, M., Grenier, A. M., Federici, B. & Rahbe, Y. 2009. Effects of Bacillus thuringiensis delta-endotoxins on the pea aphid (Acyrthosiphon pisum). Appl Environ Microbiol, 75, 4897-4900.
Promdonkoy, B. & Ellar, D. J. 2000. Membrane pore architecture of a cytolytic toxin from Bacillus thuringiensis. Biochem J, 350 Pt 1, 275-282.
Promdonkoy, B. & Ellar, D. J. 2005. Structure-function relationships of a membrane pore forming toxin revealed by reversion mutagenesis. Mol Membr Biol, 22, 327-337.
Promdonkoy, B., Promdonkoy, P., Audtho, M. et al. 2003. Efficient expression of the mosquito larvicidal binary toxin gene from Bacillus sphaericus in Escherichia coli. Curr Microbiol, 47, 383-387.
Promdonkoy B., Rungrod A., Promdonkoy P., Pathaichindachote W., Krittanai C., and Panyim S. (2000) Amino acid substitutions in αA and αC of Cyt2Aa2 alter hemolytic activity and mosquito-larvicidal specificity. J. Biotechnol. 133:287-293
Promdonkoy B., Rungrod A., Promdonkoy P., Pathaichindachote W., Krittanai C., and Panyim S. (2008) Amino acid substitutions in aA and aC of Cyt2Aa2 alter hemolytic activity and mosquito-larvicidal specificity. J. Biotechnol. 133:287-293
Quisenberry, S. S. & Schotzko, D. J. 1994. Russian wheat aphid (Homoptera: Aphididae) population development and plant damage on resistant and susceptible wheat. J. Econ. Entomol., 87, 1761-1768.
Ragsdale, D. W., Mccornack, B. P., Venette, R. C. et al. 2007. Economic threshold for soybean aphid (Hemiptera: Aphididae). J Econ Entomol, 100, 1258-1267.
Ragsdale, D. W., Voegtlin, D. J. & O'neil, R. J. 2004. Soybean aphid biology in North America. Anals Entomol. Soc. Amer., 97, 204-208.
Raps, A., Kehr, J., Gugerli, P., Moar, W. J., Bigler, F. & Hilbeck, A. 2001. Immunological analysis of phloem sap of Bacillus thuringiensis corn and of the nontarget herbivore Rhopalosiphum padi (Homoptera: Aphididae) for the presence of Cry1Ab. Mol Ecol, 10, 525-533.
Rouf Mian M A, Kang S T, Beil S E, Hammond R B. 2008. Genetic linkage mapping of the soybean aphid resistance gene in PI 243540. Theor Appl Genet. 117: 955-62.
Schnepf, E., Crickmore, N., Van R1e, J. et al. 1998. Bacillus thuringiensis and its pesticidal crystal proteins. Microbiol Mol Biol Rev, 62, 775-806.
Shelton, A. M., Zhao, J. Z. & Roush, R. T. 2002. Economic, ecological, food safety, and social consequences of the deployment of bt transgenic plants. Annu Rev Entomol, 47, 845-881.
Sylvester, E. S. 1989. Viruses transmitted by aphids. In Aphids. Their biology, natural enemies and control, Vol. C (Eds. Minks, A. K. & Harrewijn, P.), pp. 65-88. Elsevier, Amsterdam.
Tapaneeyakorn S, Pornwiroon W, Katzenmeier G, Angsuthanasombat C. 2005. Structural requirements of the unique disulphide bond and the proline-rich motif within the alpha 4-alpha 5 loop for larvicidal activity of the Bacillus thuringiensis Cry4Aa delta-endotoxin. Biochem. Biophys. Res. Commun. 330: 519-525
Terradot, L., M. Souchet, et al. (2001). “Analysis of a three-dimensional structure of Potato leafroll virus coat protein obtained by homology modeling.” Virology 286(1): 72-82.
Thackray, D. J., Wratten, S. D., Edwards, P. J. & Niemeyer, H. M. 1990. Resistance to the aphids Sitobion avenae and Rhopalosiphum padi in Poaceae in relation to hydroxamine acid. Ann Appl Biol, 116, 573-582.
Torrance, L. (1992). “Analysis of epitopes on potato leafroll virus capsid protein.” Virology 191(1): 485-489.
Walter, E. V. & Brunson, A. M. 1946. Selection for aphid resistance within inbred lines of maize. J. Am. Soc. Agron., 38, 974-977.
Walters, F. S. & English, L. H.1995. Toxicity of Bacillus thuringiensis delta-endotoxins toward the potato aphid in an artificial diet bioassay. Ent. Exp. & Appl., 77, 211-216.
Walters, F. S., Kulesza, C. A., Phillips, A. T. & English, L. H.1994. A stable oligomer of Bacillus thuringiensis delta-endotoxin, CryIIIA. Insect Biochem. Mol. Biol., 24, 963-968.
Wang, E., Wang, R., Deparasis, J., Loughrin, J. H., Gan, S. & Wagner, G. J. 2001. Suppression of a P450 hydrolylase gene in plant trichome glands enhances natural-product-based aphid resistance. Nature Biotechnol., 19, 371-374.
Zhang, X., Candas, M., Griko, N. B., Taussig, R. & Bulla, L. A., Jr. 2006. A mechanism of cell death involving an adenylyl cyclase/PKA signaling pathway is induced by the Cry1Ab toxin of Bacillus thuringiensis. Proc Natl Acad Sci USA, 103, 9897-9902.
Zhuang M, Oltean D I, Gomez I, Pullikuth A K, Soberon M, Bravo A, Gill S S. 2002. Heliothis virescens and Manduca sexta lipid rafts are involved in Cry1A toxin binding to the midgut epithelium and subsequent pore formation. J. Biol. Chem. 277: 13863-72

1. A chimeric insecticidal protein comprising an insect toxic portion and at least one target insect gut binding peptide or target insect gut binding peptide multimer portion, wherein said gut binding peptide is characterized by an amino acid sequence conforming to the consensus sequence of SEQ ID NO:21 (Xaa₁-Xaa₂-Cys-Ser-Xaa6-Xaa6-Tyr-Pro-Xaa₃-Ser-Xaa₄-Cys-Xaa₅-Xaa₆, wherein Xaa₁ and Xaa₆, independently of one another, can be any amino acid or no amino acid; Xaa₂ is Thr or Gly; Xaa₃ is Arg or Ser; Xaa₄ is Asp or Glu or Pro; Xaa₅ is Met or Gln, Xaa₆ is Lys or Ala and Xaa₇ is Arg or Ala).

2. The chimeric insecticidal protein of claim 1, wherein the gut binding peptide or peptide multimer portion comprises an amino acid sequence selected from the group SEQ ID NO:1, SEQ ID NO:2, SEQ ID NO:3, SEQ ID NO:4, SEQ ID NO:5, SEQ ID NO;6, SEQ ID NO:7, SEQ ID NO:9, SEQ ID NO:10, SEQ ID NO:11, SEQ ID NO:12, SEQ ID NO:13, SEQ ID NO:14, SEQ ID NO:15, SEQ ID NO:16, SEQ ID NO:17, SEQ ID NO:17, SEQ ID NO:18, SEQ ID NO:20, or a peptide matching consensus sequence SEQ ID NO:21.

3. The chimeric insecticidal protein of claim 1 wherein the insect toxic portion is a Cyt2A or a Cry4A insecticidal toxin of Bacillus thuringiensis.

4. The chimeric insecticidal protein of claim 3, wherein the Cyt2A protein portion has the amino acid sequence set forth in SEQ ID NO:25 or an amino acid sequence with at least 85% amino acid sequence identity thereto.

5. The chimeric insecticidal protein of claim 4 which is CGAL1 (SEQ ID NO:30), CGAL3 (SEQ ID NO:32) or CGAL4 (SEQ ID NO:34) or CGSL1 (SEQ ID NO:35), or CGSL 4 (SEQ ID NO:38).

6. The chimeric insecticidal protein of claim 3, wherein the Cry4A protein portion is derived from Cry4Aa, optionally with the gut binding peptide as an N-terminal or C-terminal extension of the Cry4Aa protein or within loops 2 or 3 of domain II or loops between β12-β13 and β15-β16 of domain III.

7. The chimeric insecticidal protein of claim 6, wherein the Cry4A protein portion has a sequence selected from the group consisting of SEQ ID NO:41, 43, 44, 45, 46, 47 AND 48 or an amino acid sequence with at least 85% amino acid sequence identity to one of the foregoing.

8. A nucleic acid molecule comprising a sequence encoding the chimeric insecticidal protein of claim 1.

9. A nucleic acid molecule according to claim 8, wherein the gut binding peptide or peptide multimer portion comprises the amino acid sequence SEQ ID NO:1.

10. A nucleic acid molecule according to claim 8, wherein the gut binding peptide or peptide multimer comprises the sequence of SEQ ID NO:17.

11. A construct comprising the sequence encoding the chimeric insecticidal protein of claim 1 is operably linked to a plant expressible promoter.

12. The construct according to claim 11, wherein the plant expressible promoter is a phloem-specific promoter.

13. The construct according to claim 11, wherein the plant expressible promoter is a leaf-specific promoter.

14. The construct according to claim 11, wherein the plant expressible promoter is a light-activated promoter or a leaf-damage activated promoter.

15. The construct according to claim 11, wherein the plant expressible promoter is a constitutive promoter.

16. A vector comprising and expressing the construct of claims 11 to 15.

17. A transformed plant containing and expressing the construct of any of claim 11.

18. A transformed plant according to claim 17, wherein the chimeric insecticidal protein is expressed in phloem tissue, leaf tissue or root tissue of the plant.

19. A method of inhibiting plant damage by a target insect, said method comprising:
providing a chimeric insecticidal protein of claim 1; and
bringing a food source comprising the chimeric insecticidal protein into contact with a target insect under conditions that allow the target insect to ingest the food,
whereby the chimeric insecticidal protein ingested by the insect inhibits feeding by or kills the target insect, resulting in reduced plant damage by the target insect.

20. The method of claim 19, wherein the target insect is a sap-sucking insect.

21. The method of claim 20, wherein the sap-sucking insect is an aphid, planthopper, thrips or whitefly, and wherein the chimeric insecticidal protein is expressed in phloem tissue of the plant.

22. The method of claim 20, wherein the food source is phloem tissue of a plant which contains and expresses the construct of claim 12 in the phloem tissue.

23. The method of claim 19, wherein the chimeric insecticidal protein comprises a gut binding peptide or peptide multimer portion which is selected to inhibit binding of a target plant virus to gut tissue of the target insect, wherein said insect that transmits said virus from plant to plant during feeding on said plant.

24. The method of claim 21, wherein the insect is an aphid, thrips, leafhopper or other sap-sucking insect.

25. A method for inhibiting transmission of a plant pathogen, wherein said plant pathogen is spread from plant to plant by sap-sucking insects, comprising the step of providing a transgenic plant expressing the chimeric insecticidal protein of claim 1, whereby the sap-sucking insects are killed and transmission of the plant pathogen is reduced.

26. The method of claim 24, wherein the plant pathogen is Pea enation mosaic virus or a soybean pathogen and the peptide, peptide multimer or the peptide comprised within the fusion protein is selected from the group of gut binding peptides having amino acid sequences given in SEQ ID NOS:1-21.

27. A host cell containing the vector of claim 16.

28. A host cell containing the construct of claim 11.

29. The host cell of claim 28, wherein said host cell is a plant cell.

30. The host cell of claim 29, wherein said plant cell is of a crop, ornamental or horticultural plant.

31. The host cell of claim 29, wherein said plant cell is of the family Rosaceae or Leguminoceae.


SEQUENCE LISTING (48 sequences)

SEQ ID NO: 1 - protein, 14 aa, Artificial Sequence
  Synthetic construct
ATCSKKYPRSPCMA

SEQ ID NO: 2 - protein, 14 aa, Artificial Sequence
  Synthetic construct
ATCSAKYPRSPCMA

SEQ ID NO: 3 - protein, 14 aa, Artificial Sequence
  Synthetic construct
ATCSKAYPRSPCMA

SEQ ID NO: 4 - protein, 15 aa, Artificial Sequence
  Synthetic construct
ATCSKKYPASPCMAS

SEQ ID NO: 5 - protein, 14 aa, Artificial Sequence
  Synthetic construct
ATCSKKYPSSDCQA

SEQ ID NO: 6 - protein, 14 aa, Artificial Sequence
  Synthetic construct
ATCSKKYPSSECMA

SEQ ID NO: 7 - protein, 14 aa, Artificial Sequence
  Synthetic construct
ATCSKKYPRSDCMA

SEQ ID NO: 8 - protein, 14 aa, Artificial Sequence
  Synthetic construct
ATCSKKYPSSPCQA

SEQ ID NO: 9 - protein, 14 aa, Artificial Sequence
  Synthetic construct
ATCSKKYPRSPCQA

SEQ ID NO: 10 - protein, 14 aa, Artificial Sequence
  Synthetic construct
AGCSKKYPRSPCMA

SEQ ID NO: 11 - protein, 12 aa, Artificial Sequence
  Synthetic construct
TCSKKYPRSPCM

SEQ ID NO: 12 - protein, 12 aa, Artificial Sequence
  Synthetic construct
TCSKKYPSSDCQ

SEQ ID NO: 13 - protein, 12 aa, Artificial Sequence
  Synthetic construct
TCSKKYPSSECM

SEQ ID NO: 14 - protein, 12 aa, Artificial Sequence
  Synthetic construct
TCSKKYPRSDCM

SEQ ID NO: 15 - protein, 12 aa, Artificial Sequence
  Synthetic construct
TCSKKYPSSPCQ

SEQ ID NO: 16 - protein, 12 aa, Artificial Sequence
  Synthetic construct
TCSKKYPRSPCQ

SEQ ID NO: 17 - protein, 12 aa, Artificial Sequence
  Synthetic construct
GCSKKYPRSPCM

SEQ ID NO: 18 - protein, 13 aa, Artificial Sequence
  Synthetic construct
TCSAKYPRSPCMA

SEQ ID NO: 19 - protein, 13 aa, Artificial Sequence
  Synthetic construct
TCSKAYPRSPCMA

SEQ ID NO: 20 - protein, 13 aa, Artificial Sequence
  Synthetic construct
TCSKKYPKSPCMA

SEQ ID NO: 21 - protein, 14 aa, Artificial Sequence
  Consensus sequence
XXCSXXYPXSXCXX

SEQ ID NO: 22 - DNA, 36 bp, Artificial Sequence
  Synthetic construct
acgtgtagtaagaagtatccgcgttctccgtgtatg

SEQ ID NO: 23 - protein, 7 aa, Artificial Sequence
  Synthetic construct
HDSSEDQ

SEQ ID NO: 24 - protein, 7 aa, Artificial Sequence
  Synthetic construct
GPSSDCQ

SEQ ID NO: 25 - protein, 5 aa, Artificial Sequence
  Synthetic construct
SSEDQ

SEQ ID NO: 26 - protein, 5 aa, Artificial Sequence
  Synthetic construct
SSDCQ

SEQ ID NO: 27 - DNA, 780 bp, Bacillus thuringiensis
atgtatactaaaaattttagtaattccagaatggaagtaaaaggtaataacggctgttctgcacctattattagaaaaccatttaaacatattgtattaacggttccatccagtgatttagataattttaatacagtcttttatgtacaaccacaatacattaatcaggctcttcatttagcaaatgcttttcaaggggctatagacccacttaatttaaatttcaattttgaaaaggcactccaaattgcaaatggtattcctaattctgcaattgtaaaaactcttaatcaaagtgttatacagcaaacagttgaaatttcagttatggttgagcaacttaaaaagattattcaagaggttttaggacttgttattaacagtactagtttttggaattcggtagaagctacaattaaaggcacatttacaaatttagacactcaaatagatgaagcatggattttttggcatagtttatccgcccacaatacaagttattattataatattttattttctattcaaaatgaagatacaggtgcagttatggcagtattacctttagcatttgaggtttctgtggatgttgaaaaacaaaaagtattattctttacaataaaagatagtgcacgatatgaagttaaaatgaaagctttgactttagttcaagctctacattcctctgatgccccaattgtagatatatttaatgttaataactataatttataccattctaatcataagattattcaaaatttaaatttatcgaattga

SEQ ID NO: 28 - protein, 259 aa, Bacillus thuringiensis
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 29 - DNA, 816 bp, Artificial Sequence
  Synthetic construct  sequence encoding CGAL1
atgtatactaaaaattttagtaattccagaatggaagtaaaaggtaataacggctgttctgcacctattattagaaaaccatttaaacatattgtattaacggttccatccagtgatttagataattttacgtgtagtaagaagtatccgcgttctccgtgtatgaatacagtcttttatgtacaaccacaatacattaatcaggctcttcatttagcaaatgcttttcaaggggctatagacccacttaatttaaatttcaattttgaaaaggcactccaaattgcaaatggtattcctaattctgcaattgtaaaaactcttaatcaaagtgttatacagcaaacagttgaaatttcagttatggttgagcaacttaaaaagattattcaagaggttttaggacttgttattaacagtactagtttttggaattcggtagaagctacaattaaaggcacatttacaaatttagacactcaaatagatgaagcatggattttttggcatagtttatccgcccacaatacaagttattattataatattttattttctattcaaaatgaagatacaggtgcagttatggcagtattacctttagcatttgaggtttctgtggatgttgaaaaacaaaaagtattattctttacaataaaagatagtgcacgatatgaagttaaaatgaaagctttgactttagttcaagctctacattcctctgatgccccaattgtagatatatttaatgttaataactataatttataccattctaatcataagattattcaaaatttaaatttatcgaattga

SEQ ID NO: 30 - protein, 271 aa, Artificial Sequence
  Synthetic construct  CGAL1
MYTKNFSNSRMEVKGNNGCSAPIIRKPFKHIVLTVPSSDLDNFTCSKKYPRSPCMNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 31 - DNA, 816 bp, Artificial Sequence
  Synthetic construct  sequence encoding CGAL3
atgtatactaaaaattttagtaattccagaatggaagtaaaaggtaataacggctgttctgcacctattattagaaaaccatttaaacatattgtattaacggttccatccagtgatttagataattttaatacagtcttttatgtacaaccacaatacattaatcaggctcttcatttagcaaatgcttttcaaggggctatagacccacttaatttaaatttcaattttgaaaaggcactccaaattgcaaatggtattcctaattctgcaattgtaaaaactcttaatcaaagtgttatacagcaaacagttgaaatttcagttatggttgagcaacttaaaaagattattcaagaggttttaggacttgttattaacagtacgtgtagtaagaagtatccgcgttctccgtgtatgactagtttttggaattcggtagaagctacaattaaaggcacatttacaaatttagacactcaaatagatgaagcatggattttttggcatagtttatccgcccacaatacaagttattattataatattttattttctattcaaaatgaagatacaggtgcagttatggcagtattacctttagcatttgaggtttctgtggatgttgaaaaacaaaaagtattattctttacaataaaagatagtgcacgatatgaagttaaaatgaaagctttgactttagttcaagctctacattcctctgatgccccaattgtagatatatttaatgttaataactataatttataccattctaatcataagattattcaaaatttaaatttatcgaattga

SEQ ID NO: 32 - protein, 271 aa, Artificial Sequence
  Synthetic construct  CGAL3
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVLGLVINSTCSKKYPRSPCMTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 33 - DNA, 816 bp, Artificial Sequence
  Synthetic construct  sequence encoding CGAL4
atgtatactaaaaattttagtaattccagaatggaagtaaaaggtaataacggctgttctgcacctattattagaaaaccatttaaacatattgtattaacggttccatccagtgatttagataattttaatacagtcttttatgtacaaccacaatacattaatcaggctcttcatttagcaaatgcttttcaaggggctatagacccacttaatttaaatttcaattttgaaaaggcactccaaattgcaaatggtattcctaattctgcaattgtaaaaactcttaatcaaagtgttatacagcaaacagttgaaatttcagttatggttgagcaacttaaaaagattattcaagaggttttaggacttgttattaacagtactagtttttggaattcggtagaagctacaattaaaggcacatttacaaatttagacactcaaatagatgaagcatggattttttggcatagtttatccgcccacaatacgtgtagtaagaagtatccgcgttctccgtgtatgacaagttattattataatattttattttctattcaaaatgaagatacaggtgcagttatggcagtattacctttagcatttgaggtttctgtggatgttgaaaaacaaaaagtattattctttacaataaaagatagtgcacgatatgaagttaaaatgaaagctttgactttagttcaagctctacattcctctgatgccccaattgtagatatatttaatgttaataactataatttataccattctaatcataagattattcaaaatttaaatttatcgaattga

SEQ ID NO: 34 - protein, 271 aa, Artificial Sequence
  Synthetic construct  CGAL4
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHSLSAHNTCSKKYPRSPCMTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 35 - protein, 264 aa, Artificial Sequence
  Synthetic construct  CGSL1
MYTKNFSNSRMEVKGNNGCSAPIIRKPFKHIVLTVPTCSKKYPRSPCMNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 36 - protein, 257 aa, Artificial Sequence
  Synthetic construct  CGSL2
MYTKNFSNSRMEVKGNNGCSAPIIRKPFKHIVLTVPSSDLDNFNTVFYVQPQYINQALHLANTCSKKYPRSPCMFEKALQIANGIPNSAIVKTLNQSVIQQTVEISVMVEQLKKIIQEVLGLVINSTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 37 - protein, 264 aa, Artificial Sequence
  Synthetic construct  CGSL3
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVTCSKKYPRSPCMTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 38 - protein, 265 aa, Artificial Sequence
  Synthetic construct  CGSL4
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHTCSKKYPRSPCMTSYYYNILFSIQNEDTGAVMAVLPLAFEVSVDVEKQKVLFFTIKDSARYEVKMKALTLVQALHSSDAPIVDIFNVNNYNLYHSNHKIIQNLNLSN

SEQ ID NO: 39 - protein, 266 aa, Artificial Sequence
  Synthetic construct  CGSL5
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHSLSAHNTSYYYNILFSIQTCSKKYPRSPCMAVMAVLPLAFEVSVDVEKQKVLFFTIKDSARYEVKMKALTLVQALHSSDAPIVDIFNVNNYNLYHSNHKIIQNLNLSN

SEQ ID NO: 40 - protein, 265 aa, Artificial Sequence
  Synthetic construct  CGSL7
MYTKNFSNSRMEVKGNNGCSAPIIRKPFKHIVLTVPSSDLDNFNTVFYVQPQYINQALHLANAFQGAIDPLNLNFNFEKALQIANGIPNSAIVKTLNQSVIQQTVEISVMVEQLKKIIQEVLGLVINSTSFWNSVEATIKGTFTNLDTQIDEAWIFWHSLSAHNTSYYYNILFSIQNEDTGAVMAVLPLAFEVSVDVEKQKVLFFTIKDSARYEVKMKALTLVQALTCSKKYPRSPCMIVDIFNVNNYNLYHSNHKIIQNLNLSN

SEQ ID NO: 41 - protein, 1180 aa, Bacillus thuringiensis
MNPYQNKNEYETLNASQKKLNISNNYTRYPIENSPKQLLQSTNYKDWLNMCQQNQQYGGDFETFIDSGELSAYTIVVGTVLTGFGFTTPLGLALIGFGTLIPVLFPAQDQSNTWSDFITQTKNIIKKEIASTYISNANKILNRSFNVISTYHNHLKTWENNPNPQNTQDVRTQIQLVHYHFQNVIPELVNSCPPNPSDCDYYNILVLSSYAQAANLHLTVLNQAVKFEAYLKNNRQFDYLEPLPTAIDYYPVLTKAIEDYTNYCVTTYKKGLNLIKTTPDSNLDGNINWNTYNTYRTKMTTAVLDVVALFPNYDVGKYPIGVQSELTREIYQVLNFEESPYKYYDFQYQEDSLTRRPHLFTWLDSLNFYEKAQTTPNNFFTSHYNMFHYTLDNISQKSSVFGNHNVTDKLKSLGLATNIYIFLLNVISLDNKYLNDYNNISKMDFFITNGTRLLEKELTAGSGQITYDVNKNIFGLPILKRRENQGNPTLFPTYDNYSHILSFIKSLSIPATYKTQVYTFAWTHSSVDPKNTIYTHLTTQIPAVKANSLGTASKVVQGPGHTGGDLIDFKDHFKITCQHSNFQQSYFIRIRYASNGSANTRAVINLSIPGVAELGMALNPTFSGTDYTNLKYKDFQYLEFSNEVKFAPNQNISLVFNRSDVYTNTTVLIDKIEFLPITRSIREDREKQKLETVQQIINTFYANPIKNTLQSELTDYDIDQAANLVECISEELYPKEKMLLLDEVKNAKQLSQSRNVLQNGDFESATLGWTTSDNITIQEDDPIFKGHYLHMSGARDIDGTIFPTYIFQKIDESKLKPYTRYLVRGFVGSSKDVELVVSRYGEEIDAIMNVPADLNYLYPSTFDCEGSNRCETSAVPANIGNTSDMLYSCQYDTGKKHVVCQDSHQFSFTIDTGALDTNENIGVWVMFKISSPDGYASLDNLEVIEEGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDIRNIIKNGDFTQGVMGWHVTGNADVQQIDGVSVLVLSNWSAGVSQNVHLQHNHGYVLGVIAKKEGPGNGYVTLMDWEENQEKLTFTSCEEGYITKTVDVFPDTDRVRIEIGETEGSFYIESIELICMNE

SEQ ID NO: 42 - DNA, 3543 bp, Bacillus thuringiensis
atgaatccttatcaaaataaaaatgaatatgaaacattaaatgcttcacaaaaaaaattaaatatatctaataattatacaagatatccaatagaaaatagtccaaaacaattattacaaagtacaaattataaagattggctcaatatgtgtcaacagaatcagcagtatggtggagattttgaaacttttattgatagtggtgaactcagtgcctatactattgtagttgggaccgtactgactggtttcgggttcacaacacccttaggacttgctttaataggttttggtacattaataccagttctttttccagcccaagaccaatctaacacatggagtgactttataacacaaactaaaaatattataaaaaaagaaatagcatcaacatatataagtaatgctaataaaattttaaacaggtcgtttaatgttatcagcacttatcataatcaccttaaaacatgggagaataatccaaacccacaaaatactcaggatgtaaggacacaaatccagctagttcattaccattttcaaaatgtcattccagagcttgtaaactcttgtcctcctaatcctagtgattgcgattactataacatactagtattatctagttatgcacaagcagcaaacttacatctgactgtattaaatcaagccgtcaaatttgaagcgtatttaaaaaacaatcgacaattcgattatttagagcctttgccaacagcaattgattattatccagtattgactaaagctatagaagattacactaattattgtgtaacaacttataaaaaaggattaaatttaattaaaacgacgcctgatagtaatcttgatggaaatataaactggaacacatacaatacgtatcgaacaaaaatgactactgctgtattagatgttgttgcactctttcctaattatgatgtaggtaaatatccaataggtgtccaatctgaacttactcgagaaatttatcaggtacttaacttcgaagaaagcccctataaatattatgactttcaatatcaagaggattcacttacacgtagaccgcatttatttacttggcttgattctttgaatttttatgaaaaagcgcaaactactcctaataattttttcaccagccattataatatgtttcattacacacttgataatatatcccaaaaatctagtgtttttggaaatcacaatgtaactgataaattaaaatctcttggtttggcaacaaatatttatatttttttattaaatgtcataagcttagataataaatatctaaatgattataataatattagtaaaatggatttttttataactaatggtactagacttttggagaaagaacttacagcaggatctgggcaaataacttatgatgtaaataaaaatattttcgggttaccaattcttaaacgaagagagaatcaaggaaaccctaccctttttccaacatatgataactatagtcatattttatcatttattaaaagtcttagtatccctgcaacatataaaactcaagtgtatacgtttgcttggacacactctagtgttgatcctaaaaatacaatttatacacatttaactacccaaattccagctgtaaaagcgaattcacttgggactgcttctaaggttgttcaaggacctggtcatacaggaggggatttaattgatttcaaagatcatttcaaaattacatgtcaacactcaaattttcaacaatcgtattttataagaattcgttatgcttcaaatggaagcgcaaatactcgagctgttataaatcttagtatcccaggggtagcagaactgggtatggcactcaaccccactttttctggtacagattatacgaatttaaaatataaagattttcagtacttagaattttctaacgaggtgaaatttgctccaaatcaaaacatatctcttgtgtttaatcgttcggatgtatatacaaacacaacagtacttattgataaaattgaatttctgccaattactcgttctataagagaggatagagagaaacaaaaattagaaacagtacaacaaataattaatacattttatgcaaatcctataaaaaacactttacaatcagaacttacagattatgacatagatcaagccgcaaatcttgtggaatgtatttctgaagaattatatccaaaagaaaaaatgctgttattagatgaagttaaaaatgcgaaacaacttagtcaatctcgaaatgtacttcaaaacggggattttgaatcggctacgcttggttggacaacaagtgataatatcacaattcaagaagatgatcctatttttaaagggcattaccttcatatgtctggggcgagagacattgatggtacgatatttccgacctatatattccaaaaaattgatgaatcaaaattaaaaccgtatacacgttacctagtaaggggatttgtaggaagtagtaaagatgtagaactagtggtttcacgctatggggaagaaattgatgccatcatgaatgttccagctgatttaaactatctgtatccttctacctttgattgtgaagggtctaatcgttgtgagacgtccgctgtgccggctaacattgggaacacttctgatatgttgtattcatgccaatatgatacagggaaaaagcatgtcgtatgtcaggattcccatcaatttagtttcactattgatacaggggcattagatacaaatgaaaatataggggtttgggtcatgtttaaaatatcttctccagatggatacgcatcattagataatttagaagtaattgaagaagggccaatagatggggaagcactgtcacgcgtgaaacacatggagaagaaatggaacgatcaaatggaagcaaaacgttcggaaacacaacaagcatatgatgtagcgaaacaagccattgatgctttattcacaaatgtacaagatgaggctttacagtttgatacgacactcgctcaaattcagtacgctgagtatttggtacaatcgattccatatgtgtacaatgattggttgtcagatgttccaggtatgaattatgatatctatgtagagttggatgcacgagtggcacaagcgcgttatttgtatgatataagaaatattattaaaaatggtgattttacacaaggggtaatggggtggcatgtaactggaaatgcagacgtacaacaaatagatggtgtttctgtattggttctatctaattggagtgctggcgtatctcaaaatgtccatctccaacataatcatgggtatgtcttaggtgttattgccaaaaaagaaggacctggaaatgggtatgtcacgcttatggattgggaggagaatcaagaaaaattgacgtttacgtcttgtgaagaaggatatattacgaagacagtagatgtattcccagatacagatcgtgtacgaattgagataggcgaaaccgaaggttcgttttatatcgaaagcattgaattaatttgcatgaacgagtga

SEQ ID NO: 43 - protein, 1180 aa, Bacillus thuringiensis
MNPYQNKNEYETLNASQKKLNISNNYTRYPIENSPKQLLQSTNYKDWLNMCQQNQQYGGDFETFIDSGELSAYTIVVGTVLTGFGFTTPLGLALIGFGTLIPVLFPAQDQSNTWSDFITQTKNIIKKEIASTYISNANKILNRSFNVISTYHNHLKTWENNPNPQNTQDVRTQIQLVHYHFQNVIPELVNSCPPNPSDCDYYNILVLSSYAHAANLHLTVLNQAVKFEAYLKNNRQFDYLEPLPTAIDYYPVLTKAIEDYTNYCVTTYKKGLNLIKTTPDSNLDGNINWNTYNTYRTKMTTAVLDVVALFPIYDVGKYPIGVQSELTREIYQVLNFEESPYKYYDFQYQEDSLTRRPHLFTWLDSLNFYEKAQTTPNNFFTSHYNMFHYTLDNISQKSSVFGNHNVTDKLKALGLATNIYIFLLNVISLDNKYLNDYNNISKMDFFITNGTRLLEKELTAGSGQITYDVNKNIFGLPILKRRENQGNPTLFPTYDNYSHILSFIKSLSIPATYKTQVYTFAWTHSSVDPKNTIYTHLTTQIPAVKANSLGTASKVVQGPGHTGGDLIDFKDHFKITCQHSNFQQSYFIRIRFASNGSANTRAVINLSIPGVAELGMALNPTFSGTDYTNLKYKDFQYLEFSNEVKFAPNQNISLVFNRSDVYTNTTVLIDKIEFLPITRSIREDREKQKLETVQQIINTFYANPIKNTLQSELTDYDIDQAANLVECISEELYPKEKMLLLDEVKNAKQLSKSRNVLQNGDFESATLGWTTSDNITIQEDDPIFKGHYLHMSGARDIDGTIFPTYIFQKIDESKLKPYTRYLVRGFVGSSKDVELVVSRYGEEIDAIMHVPADLNYLYPSTCDCEASNRCETSAVPANIGNTSDMLYSCQYDTGKKHVVCQDSHQFSFTIDTGALDTNENIGVWVMFKISSPDGYASLDNLEVIEEGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDIRNIIKNGDFTQGVMGWHVTGNADVQQIDGVSVLVLFNWSAGVSQNVHLHHNHGYVLGVIAKKEGPGNGYVTLMDWEENQEKLTFTSCEEGYITKTVDVFPDTDRVRIEIGETEGSFYIESIDLICMNE

SEQ ID NO: 44 - protein, 1180 aa, Bacillus thuringiensis
MNPYQNKNEYETLNASQKKLNISNNYTRYPIENSPKQLLQSTNYKDWLNMCQQNQQYGGDFETFIDSGELSAYTIVVGTVLTGFGFTTPLGLALIGFGTLIPVLFPAQDQSNTWSDFITQTKNIIKKEIASTYISNANKILNRSFNVISTYHNHLKTWENNPNPQNTQDVRTQIQLVHYHFQNVIPELVNSCPPNPSDCDYYNILVLSSYAQAANLHLTVLNQAVKFEAYLKNNRQFDYLEPLPTAIDYYPVLTKAIEDYTNYCVTTYKKGFNLIKTTPDSNLDGNINWITYNTYRTKMTTAVLDVVALFPIYDVGKYPIGVQSELTREIYQVLNFEESPYKYYDFQYQEDSLTRRPHLFTWLDSLNFYEKAQTTPNNFFTSHYNMFHYTLDNISQKSSVFGNHNVTDKLKALGLATNIYIFLLNVISLDNKYLNDYNNISKMDFFITNGTRLLEKELTAGSGQITYDVIKNIFGLPILKRRENQGNPTFFPTYDNYSHILSFIKSLSIPATYKTQVYTFAWTHSSVDPKNTIYTHLTTQIPAVKANSLGTASKVVQGPGHTGGDLIDFKVHFKITCQHSNFQQSYFIRIRFASNGSANTRAVINLSIPGVAELGMALNPTFSGTDYTNLKYKDFQYLEFSNEVKFAPNQNISLVFNRSDVYTNTTVLIDKIEFLPITRSIREDREKQKLETVQQIINTFYANPIKNTLQSELTDYDIDQAANLVECISEELYPKEKMLLLDEVKNAKQLSKSRNVLQNGDFESATLGWTTSDNITIQEDDPIFKGHYLHMSGARDIDGTIFPTYIFQKIDESKLKPYTRYLVRGFVGSSKDVELVVSRYGEEIDGIMHVPADLNYLYPSTCDCEASNRCETSAVPANIGNTSDMLYSCQYDTGKKHVVCQDSHQFIFTIDTGALDTNENIGVWVMFKISSPDGYASLDNLEVIEEGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDIRNIIKNGDFTQGVMGWDVTGNADVQQIDGVSVLVLFNWSAGVSQNVHLHHNHGYVLGVIAKKEGPGNGYVTLMDWEENQEKLTFTSCEEGYITKTVDVFPDTDRVRIEIGETEGSFYIESIDLICMNE

SEQ ID NO: 45 - protein, 1180 aa, Bacillus thuringiensis
MNPYQNKNEYETLNASQKKLNISNNYTRYPIENSPKQLLQSTNYKDWLNMCQQNQQYGGDFETFIDSGELSAYTIVVGTVLTGFGFTTPLGLALIGFGTLIPVLFPAQDQSNTWSDFITQTKNIIKKEIASTYISNANKILNRSFNVISTYHNHLKTWENNPNPQNTQDVRTQIQLVHYHFQNVIPELVNSCPPNPSDCDYYNILVLSSYAHAANLHLTVLNQAVKFEAYLKNNRQFDYLEPLPTAIDYYPVLTKAIEDYTNYCVTTYKKGLNLIKTTPDSNLDGNINWNTYNTYRTKMTTAVLDVVALFPIYDVGKYPIGVQSELTREIYQVLNFEESPYKYYDFQYQEDSLTRRPHLFTWLDSLNFYEKAQTTPNNFFTSHYNMFHYTFDNISQKSSVFGNHNVTDKLKALGLATNIYIFLLNVISLDNKYLKDYNNISKMDFFITNGTRLWEKELTAGSGQITYDVNKNIFGLPILKRRENQGNPTLFATYDNYSHILSFIKSLSIRATYKTQVYTFAWTHSSVDPKNTIYTHLTTQIPAVKANSLGTASKGVQGPGHTGGDLIDFKDHFKITCQHSNFQQSYFIRIRFASNGSANTRAVINLSIPGVAELGMALNPTFSGTDYTNLKYKDFQYLEFSNEVKFAPNQNISLVFNRSDVYTNTTVLIDKIEFLPITRSIREDREKQKLETVQQIINTFYANPIKNTLQSELTDYDIDQAANLVECISEELYPKEKMLLLDEVKNAKQLSKSRNVLQNGDFESATLGWTKSDNITIQEDDPIFKGHYLHRSGARDIDGTIFPTYIFQKIDESKLKPYTRYLVRGFVGSSKDVELVVSRYGEEIDAIMHFPADLNYLYPSTCDCEASNRCETSAVPANIGNTSDMLYSCQYDTGKKHVVCQDSHQFSFTIDTGALDTNENIGVWVMFKISSPDGYASLDNLEVIEEGPIDGEALSRVKHMEKKWNDQMEAKRSETQQAYDVAKQAIEALFTNVQDEALQFDTTLAQIQYAEYLVQSIPYVYNDWLSDVPGMNYDIYVELDARVAQARYLYDIRNIIKNGDFTQGVMGWHVTGNADVQQIDGVSVLVLFNWRAGVSQNVHLHHNHGYVLGVIAKKEGPGNGYVTLMDWEENQEKLTFTSCEEGYITKTVDVFPDTDRVRIEIGETEGSFYIESIDLICMNE

SEQ ID NO: 46 - protein, 1180 aa, Bacillus thuringiensis; identical to SEQ ID NO: 45

SEQ ID NO: 47 - protein, 1180 aa, Bacillus thuringiensis
MNPYQNKNEYETLNASQKKLNISNNYTRYPIENSPKQLLQSTNYKDWLNMCQQNQQYGGDFETFIDSGELSAYTIVVGTVLTGFGFTTPLGLALIGFGTLIPVLFPAQDPSNTWSDFITQTKNIIKKEIASTYISNANKILNRSFNVISTYHNHLKTWENNPNPQNTQGVRTQIQLVHYHFQNVIPELVNSCPPNPSDCDYYNILVLSSYAHAANLHLTVLNQAVNFEAYLKNNRQFDYLEPLPTAIDYYPVLTKAIEDYTNYCVTTYKKGLNLIKTTPDSNLDGNINWNTYNTYRTKMTTAVLDVVALFPIYDVGKYPIGVQSELTREIYQVLNFEESPYKYYDFQYQEDSLTRRPHLFTWLDSLNFYEKAQTTPNNFFTSHYNMFHYTLDNISQKSSVFGNHNETDKLKALGLATNIYIFLLNVISLDNKYLNDYNNISKMDFFITNGTRLLEKELTAGSGQITYDVNKNIFGLPILKRRENQGNPTLFPTYDNYSHILSFIKSLSIPATYKTQVYTFAWTHSSVDPKNTIYTHLTTQIPAVKANSLGTASKVVQGPGHTGGDLIDFKDHFKITCQHSNFQQSYFIRIRFASNGSANTRAVINLSIPGVAELGMALNPTFSGTDYTKLKYKDFQYLEFSNEVKFAPNQNISLVFNRSDVYTNTTVLIDKIEFLPITRSIREDREKQKLETVQQIINTFYANPIKNTLQSELTDYDIDQAANLVECISEELYPKEKMLLLDEVKNAKQLSKSRNVLQNGDFEPATLGWTTSDNITIQEDDPIFKGHYLHMSGARDIDGTIFPTYIFQKIDESKLKPYTRYLVRGFVGSSKDVELVVSRYGEEIDAIMHVPADLNYLYPSTCDCEASNRCETSAVPANIGNTSDMLYSCQYDTGKKHVVCQDSHQFSFTIDTGALDTNENIGVWVMFKISSPDGYASLDNLEVIEEGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDIRNIIKNGDFTQGVMGWHVTGNADVQQIDGVSVLVLFNWSAGVSQNVHLHQNHGYVLGVIAKKEGPGNGYVTLMDWEENQEKLTFTSCEEGYITKTVDVFPDTDRVRIEIGETEVSFYIESIDLICMNE

SEQ ID NO: 48 - protein, 1180 aa, Bacillus thuringiensis
MNPYQNKNEYETLNASQKKLNISNNYTRYPIENSPKQLLQSTNYKDWLNMCQQNQQYGGDFETFIDSGELCAYTIVVGTVLTGFGFTTPLGLALIGFGTLIPVLFPAQDQSNTWSDFITQTKNIIKKEIASTYISNANKILNRSFNVISTYHNHLKTWENNPNPQNTQDVRTQIQLVHYHFQNVIPELVNSCPPNPSDCDYYNILVLSSYAHAANLHLTVLNQAVKFEAYLKNNRQFDYLEPLPTAIDYYPVLTKAIEDYTNYCVTTYKKGLNLIKTTPDSNLDGNINWNTYNTYRTKMTTAVLDVVALFPIYDVGKYPIGVQSELTREIYQVLNFEESPYKYYDFQYQEDSLTRRPHLFTWLDSLNFYEKAQTTPNNFFTSHYNMFLYTLDNISQKSSVFGNHNVTDKLKALGLATNIYIFLLNVISLDNKYLNDYNNISKMDFFITNGTRLLEKELTAGSGQITYDVNKNIFGLPILKRRENQGNPTLFPTYDNYSHILSFIKSLSIPETYKTQVYTFAWTHSSVDPKNTIYTHLTTQIPAVKANSLGTASKVVQGPGHTGGDLIDFKDHFKITCQHSNFQQSYFIRIRFASNGSANTRAVINLSIPRVAELGMALNPTFSGTDYTNLKYKDFQYLEFSNEVKFAPNQNISLVFNRSDVYTNTTVLIDKIEFLPITRSIREDREKQKLETVQQIINTFYANPIKNTLQSELTDYDIDQAANLVECISEELYPKEKMLLLDEVKNAKQLSKSRNVLQNGDFESATLGWTTSDNITIQEDDPIFKGHYLHMSGARDIDGTIFPTYIFQKIDESKLKPYTRYLVRGFVGSSKDVELVVSRYGEEIDAIMHVPADLNYLYPSTCDCEASNRCETSAVPANIGNTSDMLYSCQYDTGKKHVVCQDSHQFSFTIDTGALDTNENIGVWVMFKISSPDGYASLDNLEVIEEGPIDGEALSRVKHMEKKWNDQMEAKRSETQQAYDVAKQAIDALFTNVQDEALQFDTTLAQIQYAEYLVQSIPYVYNDWLSDVPGMNYDIYVELDARVAQARYLYDIRNIIKNGDFTQGVMGWHVTGNADVQQIDGVSVLVLFNWSAGVSQNVHLHHNHGYVLGVIAKKEGPGNGYVTLMDWEENQEKLTFTSCEEGYITKTVDVFPDTDRVRIEIGETEGSFYIESIDLICMNE